(19) United States
(12) Patent Application Publication
(10) Pub. No.: US 2023/0091848 A1
(43) Pub. Date: Mar. 23, 2023

(54) DISEASE DETECTION AND TREATMENT BASED ON PHENYLACETYL GLUTAMINE LEVELS

(71) Applicant: The Cleveland Clinic Foundation, Cleveland, OH (US)

(72) Inventors: Stanley L. Hazen, Pepper Pike, OH (US); Ina Nemet, Lyndhurst, OH (US); Prasenjit Saha, Cleveland, OH (US)

(21) Appl. No.: 17/795,465

(22) PCT Filed: Feb. 4, 2021
(86) PCT No.: PCT/US21/16508

§ 371 Date: Jul. 26, 2022

Related U.S. Application Data
(60) Provisional application No. 62/970,480, filed on Feb. 5, 2020.

(51) Int. Cl. G01N 33/50; G01N 33/574; A61K 31/19

(52) U.S. Cl. CPC G01N 33/5038; G01N 33/574; A61K 31/19; G01N 2800/52

(57) ABSTRACT

The present invention relates to systems, kits, and methods for identifying subjects with increased levels of phenylacetyl glutamine (PAG) or the combination of PAG and trimethylamine-n-oxide (TMAO) and/or N6-trimethyl-lysine (TML), and/or PSA, pp and/or betaine, and/or choline, as well as methods of determining risk of disease (e.g., CVD, heart failure, asthma, diabetes, thrombosis, and lethal prostate cancer) based on such levels. In certain embodiments, the subjects are free of chronic kidney disease and/or have type II diabetes. In particular embodiments, subjects are treated with a therapeutic, such as a beta-adrenergic blocking agent, an alpha 2 adrenergic receptor agonist, an alpha 2 adrenergic receptor antagonist, an antibiotic or antibiotic cocktail, or a prostate cancer therapeutic. In certain embodiments, the subject is treated with a procedure such as brachytherapy, radiation therapy, or prostatectomy.

### STATEMENT REGARDING FEDERAL FUNDING

This invention was made with government support under grant number HL 147823 awarded by the National Institutes of Health. The government has certain rights in the invention.

The present application claims priority to U.S. Provisional application Ser. No. 62/970,480, filed Feb. 5, 2020, which is herein incorporated by reference.

### FIELD OF THE INVENTION

The present invention relates to systems, kits, and methods for identifying subjects with increased levels of phenylacetyl glutamine (PAG) or the combination of PAG and trimethylamine-n-oxide (TMAO) and/or N6-trimethyl-lysine (TML), and/or PSA, and/or betaine, and/or choline, as well as methods of determining risk of disease (e.g., CVD, heart failure, asthma, diabetes, thrombosis, prostate cancer, and lethal prostate cancer) based on such levels. In certain embodiments, the subjects are free of chronic kidney disease and/or have type II diabetes. In particular embodiments, subjects are treated with a therapeutic, such as a beta-adrenergic blocking agent, an alpha 2 adrenergic receptor agonist, an alpha 2 adrenergic receptor antagonist, an antibiotic or antibiotic cocktail, or other prostate cancer therapeutic. In certain embodiments, the subject is treated with a procedure such as brachytherapy, radiation therapy, or prostatectomy.

### BACKGROUND

Cardiovascular disease (CVD) remains the leading cause of death and morbidity in Western countries and new therapeutic targets that contribute to CVD development and progression are needed. Subjects with type 2 diabetes (T2DM) are at markedly higher risk for development of CVD and its major adverse consequences (MACE, major adverse cardiac events=myocardial infarction (MI), stroke or death), suffer poorer prognosis and outcomes, and traditional CVD risk factors fail to adequately account for the heightened risks observed amongst subjects with T2DM. Interestingly, the degree of blood-glucose control within T2DM is a poor indicator of incident CVD risks, and numerous anti-diabetes medications lower plasma glucose levels without significantly impacting CVD development or MACE risks (Action to Control Cardiovascular Risk in Diabetes Study et al., 2008) (Duckworth et al., 2009; Gerstein et al., 2001; Piarulli et al., 2009). These observations indicate that metabolic derangements distinct from glucose related pathways occur with T2DM that contribute to the heightened CVD risks observed in this disorder.

### SUMMARY OF THE INVENTION

The present invention relates to systems, kits, and methods for identifying subjects with increased levels of phenylacetyl glutamine (PAG) or the combination of PAG and trimethylamine-n-oxide (TMAO) and/or N6-trimethyl-lysine (TML), and/or PSA, and/or betaine, and/or choline, as well as methods of determining risk of disease (e.g., CVD, heart failure, asthma, diabetes, thrombosis, prostate cancer, and lethal prostate cancer) based on such levels. In certain embodiments, the subjects are free of chronic kidney disease and/or have type II diabetes. In particular embodiments, subjects are treated with a therapeutic, such as a beta-adrenergic blocking agent, an alpha 2 adrenergic receptor agonist, an alpha 2 adrenergic receptor antagonist, an antibiotic or antibiotic cocktail, or other prostate cancer therapeutic. In certain embodiments, the subject is treated with a procedure such as brachytherapy, radiation therapy, or prostatectomy.

In some embodiments, provided herein are methods of performing an activity based on the level of at least phenylacetyl glutamine (PAG) in a sample from a subject comprising: a) determining the level of at least one compound in a sample from a subject, wherein the at least one compound comprises PAG, and wherein optionally the subject is chronic kidney disease (CKD) free and/or has type II diabetes; and b) performing at least one of the following activities: i) identifying increased levels of the at least one compound in the sample compared to control levels, and treating the subject with: A) a first agent or first procedure that treats cardiovascular disease (CVD), asthma, heart failure, and/or thrombosis, wherein the subject is CKD free and/or has diabetes, B) a second agent selected from: a beta-adrenergic blocking agent, an alpha 2 adrenergic receptor agonist, an alpha 2 adrenergic receptor antagonist, and an antibiotic or antibiotic cocktail and/or C) a third agent or second procedure selected from: a prostate cancer therapeutic, brachytherapy, radiation therapy, and prostatectomy; ii) generating and/or transmitting a report that indicates that levels of the at least one compound are elevated in the sample compared to control levels, and that the subject has, or is at risk for, CVD, asthma, prostate cancer (e.g., lethal prostate cancer), thrombosis, and/or heart failure and is treatable by: A) the beta-adrenergic blocking agent, and/or B) the alpha 2 adrenergic receptor agonist, and/or C) the an alpha 2 adrenergic receptor antagonist and/or D) the antibiotic or antibiotic cocktail; and/or E) said third agent or second procedure; iii) generating and/or transmitting a report that indicates that levels of the at least one compound are elevated in the sample compared to control levels, and that the subject is in need of the first agent, the second agent, and/or the procedure, and/or the third agent or second procedure; iv) generating and/or transmitting a report for a subject that is CKD free and/or has diabetes, wherein the report indicates that levels of the at least one compound are elevated in the sample compared to control levels, and that the subject has, or is at risk for, cardiovascular disease, asthma, thrombosis, prostate cancer, lethal prostate cancer, and/or heart failure; and v) characterizing the subject who is CKD free and/or has diabetes as having, or at risk for, CVD, thrombosis, and/or heart failure, or characterizing the subject as having, or at risk for, lethal prostate cancer, based on finding elevated levels of the at least one compound in the sample compared to a control levels.

In certain embodiments, provides herein are methods of treatment comprising: a) identifying a subject as having increased levels of at least one compound compared to control levels, wherein the at least one compound comprises phenylacetyl glutamine (PAG); and b) treating the subject with at least one of the following: i) a first agent or procedure that treats cardiovascular disease (CVD), heart failure, asthma, and/or thrombosis, wherein the subject is chronic kidney disease (CKD) free and/or has diabetes, ii) a second agent selected from: a beta-adrenergic blocking agent, an alpha 2 adrenergic receptor agonist, and an alpha 2 adrenergic receptor antagonist, iii) an antibiotic or antibiotic cocktail, wherein the subject does not have an active infection, and iv) a third agent or second procedure selected from: a prostate cancer therapeutic, brachytherapy, radiation therapy, and prostatectomy. In certain embodiments, the identifying comprises receiving a report that the subject has increased PAG levels compared to a control.

In particular embodiments, provided herein are systems and kits comprising: a) a report for a subject with cardiovascular disease, heart failure, asthma, prostate cancer, lethal prostate cancer, and/or thrombosis, wherein the report indicates that the patient has elevated levels of at least one compound, wherein the at least one compound comprises phenylacetyl glutamine (PAG); and b) at least one of the following: i) a first agent that treats CVD, heart failure, asthma, and/or thrombosis, wherein the subject is chronic kidney disease (CKD) free and/or has diabetes, ii) a second agent selected from: a beta-adrenergic blocking agent, an alpha 2 adrenergic receptor agonist, and an alpha 2 adrenergic receptor antagonist, iii) an antibiotic or antibiotic cocktail, wherein the subject does not have an active infection, and iv) a prostate cancer therapeutic.

In some embodiments, provided herein are methods of detecting at least three compounds in a sample comprising: a) obtaining a sample, wherein said sample is from a human subject; and b) treating said sample under conditions such that the concentration of at least the following three compounds is determined: phenylacetyl glutamine (PAG), N6-trimethyl-lysine (TML), and trimethylamine N-oxide (TMAO).

In some embodiments, the methods further comprise: c) graphically displaying said subject's risk of having a disease as higher than normal if all three are present: i) said level of PAG in said sample is higher than a control PAG value from the general population or disease free group; ii) said TMAO level in said sample is higher than a control TMAO value from the general population or a disease free group; and iii) said TML level is in said sample is higher than a control TML value from the general population or a disease free group; and wherein said disease is selected from the group consisting of heart failure, cardiovascular disease, kidney disease, asthma, or thrombosis. In certain embodiments, the methods further comprise: c) graphically displaying said subject's risk of having a disease as higher than normal if all three are present: i) said level of PAG in said sample is higher than a first minimum value, wherein said minimum value is at least 3.8 or 4.9 μM, ii) said TMAO level is higher than a second minimum value, wherein said second minimum value is at least 2.2 or 5.0 μM; and iii) said TML level is higher than a third minimum value, wherein said second minimum value is at least 0.4 or 0.6 μM; and wherein said disease is selected from the group consisting of heart failure, cardiovascular disease, kidney disease, asthma, or thrombosis.

In some embodiments, the at least one compound further comprises trimethylamine-n-oxide (TMAO). In additional embodiments, the at least one compound further comprises N6-trimethyl-lysine (TML). In further embodiments, the at least one compound further comprises TMAO and TML. In some embodiments, the at least one compound further comprises PSA, choline, and/or betaine, and said subject has risk of, or has, prostate cancer. In additional embodiments, the at least one compound further comprises trimethylamine-n-oxide (TMAO) and/or N6-trimethyl-lysine (TML) and/or PSA and/or choline, and/or betaine.

In certain embodiments, wherein: i) the subject has symptoms of, is at risk for, or has heart failure, asthma, or cardiovascular disease, and wherein the at least one compound further comprises N6-trimethyl-lysine (TML) and/or TMAO, or ii) the subject has (or is at risk for) prostate cancer, and said at least one compound further comprises PSA, choline, and/or betaine. In certain embodiments, the subject has prostate cancer or lethal prostate cancer and is treated with the agent selected from: a beta-adrenergic blocking agent, an alpha 2 adrenergic receptor agonist, an alpha 2 adrenergic receptor antagonist, and an antibiotic or antibiotic cocktail. In some embodiments, the subject is a human with prostate cancer, cardiovascular disease, asthma, or heart failure.

In particular embodiments, the sample is selected from the group consisting of: a plasma sample, a serum sample, a whole blood sample, and a urine sample. In other embodiments, the determining comprises detecting the at least one compound with an analytical device selected from: a mass spectrometer, NMR spectrometer, and a UV/Vis spectrometer, anti-PAG antibody based detection system (e.g., ELISA or competitive ELISA, etc.). In additional embodiments, the determining comprises contacting the sample with an anti-PAG antibody or PAG binding fragment thereof.

In some embodiments, the beta-adrenergic blocking agent is selected from: acebutolol hydrochloride, atenolol, betaxolol hydrochloride, bisoprolol fumarate, carteolol hydrochloride, esmolol hydrochloride, metoprolol, penbutolol sulfate, nadolol, nebivolol, pindolol, propranolol, timolol maleate, sotalol hydrochloride, carvedilol, and labetalol hydrochloride. In further embodiments, the alpha 2 adrenergic receptor agonist is selected from: Clonidine, Dexmedetomidine, Fadolmidine, Guanfacine, Guanabenz, Guanoxabenz, Guanethidine, Xylazine, Tizanidine, Medetomidine, Methyldopa, Methylnorepinephrine, Norepinephrine, (R)-3-nitrobiphenyline, amitraz, Detomidine, Lofexidine,and Medetomidine. In other embodiments, the alpha 2 adrenergic receptor antagonist is selected from: atipamezole, efaroxan, idazoxan, yohimbine, rauwolscine, and phentolamine. In certain embodiments, the first agent is selected from the group consisting of: an anticoagulant, an antiplatelet agent, an ACE Inhibitor, an angiotensin II receptor blocker, an angiotensin-receptor neprilysin inhibitor, a calcium channel blocker, a cholesterol-lowering medication, a statin, a digitalis preparation, a diuretic, and a vasodilator. In certain embodiments, the antibiotic is at least one antibiotic selected from the group consisting of: metronidazole, ciprofloxacin, neomycin, vancomycin, amoxicillin, and a broad spectrum antibiotic; and wherein the subject does not have an active infection. In particular embodiments, the subject is a human.

In certain embodiments, provided herein are methods of detecting at least one compound in a sample comprising: a) obtaining a sample, wherein the sample is from a human subject who is chronic kidney disease (CKD) free and/or has diabetes; and b) treating the sample under conditions such that the concentration of at least one compound is determined, wherein the at least one compound comprises phenylacetyl glutamine (PAG). In some embodiments, the concentration of the PAG in the sample is determined by mass spectrometry. In further embodiments, the sample is a plasma or serum sample. In other embodiments, the at least one compound further comprises trimethylamine-n-oxide (TMAO) and/or N6-trimethyl-lysine (TML). In certain embodiments, the sample is from a subject with, or at risk for, prostate cancer, lethal prostate cancer, cardiovascular disease, asthma, heart failure, or thrombosis.

In some embodiments, provided herein are methods of detecting phenylacetyl glutamine (PAG) in a serum or plasma sample comprising: a) obtaining a plasma or serum sample, wherein the plasma or serum sample is from a human subject who is chronic kidney disease (CKD) free and/or has type II diabetes; b) introducing at least a portion of the plasma or serum sample into an analytical device under conditions such that the concentration of PAG present in the plasma or serum sample is determined, wherein the analytical device comprises: i) an NMR spectrometer, a UV/Vis spectrometer, or a mass spectrometer, ii) equipment to provide physical separation of the PAG prior to determining the concentration; and iii) an anti-PAG antibody based detection system (e.g., ELISA or competitive ELISA, etc), and c) graphically displaying the subject's risk of having a disease as higher than normal if the level of PAG in the plasma or serum sample is higher than a first minimum value, wherein the minimum value is at least 3.8 or 4.9 μM, wherein the disease is selected from the group consisting of heart failure, cardiovascular disease, asthma, or thrombosis.

In certain embodiments, the methods further comprise determining the level of trimethylamine N-oxide (TMAO) in a sample from a subject, and graphically displaying the subject's risk of the disease as higher than normal when the TMAO level is higher than a second minimum value, wherein the second minimum value is at least 2.2 μM (e.g., at least 2.5, 3.0, 3.5, or 4.0 or 5.0 μM).

In some embodiments, the methods further comprise determining the level of N6-trimethyl-lysine (TML) in a sample from a subject, and graphically displaying the subject's risk of the disease as higher than normal when the TML level is higher than a second minimum value, wherein the second minimum value is at least 0.4 μM (e.g., 0.5, 0.6, 0.7, or 0.8 μM).

In certain embodiments, provided herein are methods of detecting phenylacetyl glutamine (PAG) in a serum or plasma sample comprising: a) obtaining a plasma or serum sample, wherein the plasma or serum sample is from a human subject who is optionally chronic kidney disease (CKD) free and/or has type II diabetes; b) introducing at least a portion of the plasma or serum sample into an analytical device under conditions such that the concentration of PAG present in the plasma or serum sample is determined, wherein the analytical device comprises: i) an NMR spectrometer, a UV/Vis spectrometer, or a mass spectrometer, ii) equipment to provide physical separation of the PAG prior to determining the concentration, and iii) an anti-PAG antibody based detection system (e.g., ELISA or competitive ELISA, etc.); and c) graphically displaying the subject's risk of having a disease as higher than normal if the level of PAG in the plasma or serum sample is higher than a first minimum value, wherein the minimum value is at least 1.0 μM or at least 3.0 μM, wherein the disease is selected from the group consisting of prostate cancer and lethal prostate cancer.

In particular embodiments, the method further comprise determining the level of choline in a sample from a subject, and graphically displaying the subject's risk of the disease as higher than normal when the choline level is higher than a second minimum value, wherein the second minimum value is at least 11.0 μM. In other embodiments, the second minimum value is at least 13.0 μM, or at least 14.0 μM, or at least 15.0 μM, or at least 17.0 μM.

In some embodiments, the methods further comprise determining the level of betaine in a sample from a subject, and graphically displaying the subject's risk of the disease as higher than normal when the betaine level is higher than a second minimum value, wherein the second minimum value is at least 35.6 μM. In further embodiments, the second minimum value is at least 42.8 μM, or at least 54.0 μM, or at least 54.7 μM, or at least 55 μM.

In certain embodiments, provided herein are methods of detecting at least three or four compounds in a sample comprising: a) obtaining a sample, wherein the sample is from a human subject; and b) treating the sample under conditions such that the concentration of at least the following three compounds is determined: phenylacetyl glutamine (PAG), choline, and betaine, and optionally a fourth compound: prostate specific antigen (PSA). In certain embodiments, the methods further comprise: c) graphically displaying the subject's risk of having a disease as higher than normal if all three are present: i) the level of PAG in the sample is higher than a control PAG value from the general population or disease free group; ii) the choline level in the sample is higher than a control choline value from the general population or a disease free group; and iii) the betaine level is in the sample is higher than a control TML value from the general population or a disease free group; and iv) and optionally, the PSA level in the sample is higher than a control PSA value from the general population or disease free group; and wherein the disease is selected from the group consisting of prostate cancer and lethal prostate cancer. In some embodiments, the methods further comprise: c) graphically displaying the subject's risk of having a disease as higher than normal if all three are present: i) the level of PAG in the sample is higher than a first minimum value, wherein the minimum value is at least 3.9 or 4.9 μM, ii) the choline level is higher than a second minimum value, wherein the second minimum value is at least 11.0 or 13.0 μM; and iii) the betaine level is higher than a third minimum value, wherein the second minimum value is at least 35.6 μM or 42.8; and wherein the disease is selected from the group consisting of prostate cancer and lethal prostate cancer.

### BRIEF DESCRIPTION OF THE FIGURES

FIG. 1. Untargeted metabolomics studies discover a metabolite with m/z of 265.1188 is associated with cardiovascular disease risk, which is subsequently identified as phenylacetylglutamine (PAGln). (A) Relative plasma levels of compound with m/z 265.1188 in sequential stable subjects undergoing elective diagnostic cardiac evaluation. Subjects (n=1,162) were divided into groups as indicated based on whether (Yes) or not (No) they were diabetic or experienced an incident major adverse cardia event (MACE: MI, stroke or death) over the 3 years follow-up. In the box-whisker plot, the upper and lower boundaries of the box represent the 25th and 75th percentiles, the median is marked by a horizontal line inside the box, while whiskers represent 10 and 90% of relative measured values. (B) Kaplan-Meier estimates and the risk of incident major averse cardiac events (MACE, MI, stroke or death) ranked by quartile of relative amounts of unknown compound from untargeted analysis with m/z 265.1188. (C) Comparison of high resolution CID spectra in positive ion mode of the metabolite m/z 265.1188 in plasma (red) and synthetic PAGln (blue). (D) Kaplan-Meier estimates and the risk of MACE ranked by quartile of PAGln levels measure by stable isotope dilution method (targeted analysis). (E) Forest plots indicating PAGln is associated with the risk of MACE by 3 years among all test subjects (n=4,000; left panel), diabetics (n=1,261; mid panel) and non-diabetics (n=2,739, right panel) according to the quartiles for PAGln level, multivariable Cox model for hazard ratio (black) included adjustments for age, gender, smoking, HDL, LDL, triglyceride level, systolic blood pressure, hyperlipidemia and C-reactive protein level (Adj, red). The 5-95% confidence intervals (CI) are indicated by the line length.

FIG. 2. Phenylacetylglutamine (“PAGln” or “PAG”) production in vivo is a microbiota-dependent process in humans and mice. (A) Levels of PAGln were measured in human plasma (i) before oral treatment with a broad spectrum antibiotic (Pre-Abx), (ii) after seven days of antibiotics regiment (Abx), and (iii) three week after antibiotic treatment ended permitting repopulation of gut microorganisms (Post-Abx). Human blood was collected from overnight fasted healthy volunteers (n=15). Kruskal-Wallis was used for the statistical analysis. (B) Levels of PAGln and PAGly in healthy human subjects (left panel), conventional mice (mid panel) and germ free (GF) mice (right panel). (C) After IP injection of phenylacetic acid (50 mg/kg), mice predominantly make PAGly (500× time more than PAGln). (D) Levels of PAGln were measured in mouse plasma (i) before use of oral broad spectrum antibiotic (Pre-Abx), (ii) after five days of antibiotics treatment (Abx) and (iii) one week post antibiotics washout permitting repopulation of gut microorganisms (Post-Abx). Mouse blood was collected from non-fasting male (n=11) and female (n=15) C57Bl/6J mice. Kruskal-Wallis test was used for the statistical analysis. (E) Mulitiorganismal production of phenylacetylglutamine (PAGln) and phenylacetylglycine (PAGly) in human and mice. Phenylacetic acid is bacterial product derived from phenylalanine which in human liver gets predominantly converted into phenylacetylglutamine (PAGln), while in mice into phenylacetylglycine (PAGly).

FIG. 3. Phenylacetylglutamine (PAGln) is associated with enhanced thrombosis potential. (A) Human platelet adhesion in whole blood to a microfluidic chip surface (f collagen coating) under physiological shear conditions f PAGln; representative images of platelet adhesion at the indicated times. (B) Adherent platelet area per μm² of chip surface. (C) Platelet rich plasma (PRP) was isolated from healthy volunteers with low plasma PAGln levels. After addition of PAGln (100 μM final, red) versus normal saline (vehicle, blue), PRP was incubated at r.t. for 30 min, and then platelet aggregometry was used to assess ADP, thrombin (TRAP 6) and collagen dose-response curves for each subject (upper panels). Aggregometry was used to assess PAGln dose-respond curves at constant concentration of agonist (ADP (2 μM), TRAP6 (7.5 μM) or collagen (0.75 μg/mL)) (bottom panels). (D-E) Thrombin induced changes in intracellular calcium concentration [Ca²⁺] in Fura 2 filled washed human platelets incubated with the different amounts of PAGln (0 (blue); 3.1(green); 10 (red); 3 3 (purple) and 100 (black) μM). (D) Representative fluorescent signal from a single subject's platelet preparation. Significance was measure with non-parametric one- or two-way ANOVA.

FIG. 4. Phenylacetylglutamine (PAGln) and phenylacetylglycine (PAGly) enhance in vivo thrombosis potential. (A-B) In vivo thrombosis potential was measured by the FeCl₃-induced carotid artery injury model after IP injection (50 mg/kg) of phenylalanine (Phe); phenylacetylglutamine (PAGln), phenylacetylglycine (PAGly). Shown are representative vital microscopy images of carotid artery thrombus formation at the indicated time points following arterial injury (A), and time to cessation of blood flow in mice from the indicated groups (B). Bar represents mean time to cessation of blood flow within the indicated group. Significance was measured with unpaired one way ANOVA. (C) Synthesis of phenylacetic acid and phenylpropionic acid by C. sporogenes ΔcutC mutant with disrupted gene for reductive metabolism of phenylalanine (ΔfldH) were compared to C. sporogenes ΔcutC mutant with disrupted gene for oxidative metabolism of phenylalanine (ΔporA). C. sporogenes mutants were incubated with synthetic ³C₉¹⁵N-phenylalanine and production of ¹³C₈-phenylacetic acid (red) and ¹³C₉-phenylpropionic acid (blue) was measured by LC-MS/MS (values were normalized by optical density (OD)). (D) Time to cessation of blood flow 24 hour post folic acid IP injection in GF mice mono-colonized with C. sporogenes ΔcutC or C. sporogenes ΔcutCΔfldH mutants for 2-7 days. In vivo thrombosis potential was measured by the FeCl₃-induced carotid artery injury model. Shown are representative vital microscopy images of carotid artery thrombus formation at the indicated time points following arterial injury (bottom), and time to cessation of blood flow in mice from the indicated groups (top). Bar represents mean time to cessation of blood flow within the indicated group.

FIG. 5. PAGln mediates cellular response through G-protein coupled receptor(s) and loss of function studies suggests that the metabolite signals via adrenergic receptors present on human platelets. (A) Dynamic mass redistribution (DMR) dose response of phenylacetylglutamine (PAGln), norepinephrine (Norepi) and phenylalanine (Phe) in MEG01 cell lines. Curves were generated quantifying the maximum DMR peaks after ligand addition. (B) DMR response of 100 μM PAGln (left), 10 μM Norepi (middle) and 10 μg/mL collagen (right) after treatment of G-protein modulators, pertussis toxin (PTX; 100 ng/mL), cholera toxin (CTX; 1 μg/mL), YM-254890 (0.5 μM) and SCH-202676 (1 μM) which masks signaling through Gαᵢ-subunit, Gas-subunit, Gα_{q}-subunit and almost all G-protein subunits respectively. Maximum response of PAGln, Norepi and collagen have been normalized to 100%. (C-D) cAMP levels in MEG01 cell lines (C) and in washed human platelets (D) after treating them with PAGln (100 μM) for 5 min, in presence of PTX (100 ng/mL), CTX (1 μg/mL), YM-254890 (1 μM) and SCH-202676 (1 μM). The value of cAMP level normalized to 100% in all the modulators treated or untreated subjects before addition of PAGln. (E) Structural analysis reveals that PAGln has core backbone structure of phenylethylamine moiety (marked in yellow) similar to certain catecholemines (Isoproterenol, Epi and Norepi). (F) DMR response of PAGln (100 μM) was measured after transfecting the cells with control scrambled siRNAs, siRNAs against α2A, α2B and β2 adrenergic receptors in MEG01 cells. Maximum response of PAGln normalized to 100%. (G) PAGln (100 μM) DMR response was quantified after treating MEG01 cells for 30 min with selective beta-2 antagonist ICI118,551 (10 μM), nonselective beta-blocker propranolol (10 μM) and nonselective alpha-2 antagonist RX821002 (10 μM). Maximum response of PAGln normalized to 100%. (H) cAMP level was measured in MEG01 cell lines after treating the cells with PAGln (100 μM) for 5 min, in presence of ICI118,551 (10 μM), propranolol (10 μM) and RX821002 (10 μM). (The value of cAMP level was normalized to 100% in all the inhibitor treated or untreated subjects before addition of PAGln). Significance was determined by two-tailed student's t-test (nonparametic-Mann Whitney test) and Kruskal-Wallis (K.W.) test for multiple comparisons. Data points represent the mean±s.e.m. (n=biological replicates).

FIG. 6. Gain of function studies confirms PAGln demonstrates cellular events through platelet's adrenergic receptors that leads to in vitro and in vivo platelet aggregation, which can be attenuated with beta-blockers. (A) PAGln (100 μM) DMR response was analyzed in parental HEK293, empty vector transfected HEK293, ADRA2A transfected HEK293 (left panel), α2B-HEK293 stable cells (middle panel) and in β2-HEK293 stable cells (right panel) after treating the cells with selective beta-2 antagonist ICI118,551 (10 μM), nonselective beta-blocker propranolol (10 μM) and nonselective alpha-2 antagonist RX821002 (10 μM) for 30 min. (PAGln response was calculated with respect to relative ATP response in all the subjects). (B) cAMP level was analyzed in parental HEK293 (green line) and in β2-HEK293 stable cell line (blue line) after treating the cells with the indicated increasing concentration of PAGln for 10 min. (C) Platelet aggregation was measured in human platelet rich plasma (PRP) in response to PAGln (100 μM) with a submaximal concentration of ADP (2 μM), in presence of nonselective beta-blocker propranolol (10 μM) and nonselective alpha-2 blocker RX821002 (10 μM). Data points represent aggregation as the percentage of maximum amplitude in PRP recovered from each human subjects. D) Representative vital microscopy images of carotid artery thrombus formation at the indicated time points following FeCl₃-induced carotid artery injury in mice i.p. injected with PAGln and fed with or without beta-blocker carvedilol (1.5 mg/kg) supplemented diet for a week. The time to complete vessel occlusion is mentioned in the respective right side panels. (E) Quantification of the aforementioned in vivo thrombosis formation following FeCl₃-induced carotid artery injury, cumulatively represented as bar graph for the indicated numbers of mice in each group. Data points represent the time to cessation of flow for each mouse subject. Respective plasma PAGln level noted for each group at the bottom. Significance was determined by nonparametic-Mann Whitney (M.W.) test for pairwise comparison and Kruskal-Wallis (K.W.) test for multiple comparison. Data points represent the mean±s.e.m. (n=biological replicates).

FIG. 7 shows a proposed multiorganismal production of phenylacetylglycine and phenylacetylglutamine.

FIG. 8. Event Rates for Major Adverse Cardiac Events at 3 Years According to High vs Low Marker Levels from Example 2.

FIG. 9. Event Rates for All-Cause Mortality at 5 Years According to High vs Low Marker Levels from Example 2.

FIG. 10. FIG. 10 shows even free survival at 3 years (FIG. 10a) and 5 years (FIG. 10b) for single and multiple markers.

FIG. 11. Gut-Microbiome-Mediated Metabolism of Trimethylamine Precursors and Select Amino Acids. (A) Precursors to trimethylamine (TMA)-including choline, carnitine, betaine, γ-butyrobetaine, and crotonobetaine—are consumed through the diet. These nutrients may be converted to other TMA precursors by the host (shown via blue arrows) or by gut microbiota (shown via green arrows). A minority of these precursor nutrients reach the large intestine and are metabolized by gut microbiota to TMA, which is ultimately absorbed by the host. Via the portal circulation, TMA reaches the liver where it is oxidized to trimethylamine N-oxide by flavin-containing monooxygenases (FMOs). TMAO then enters the systemic circulation where it may exert its physiologic effects on its human host. (B) Amino acids-including phenylalanine, glycine, and tyrosine—are ingested through dietary protein. In the stomach and small intestine, these amino acids are liberated by proteases. A minority of these amino acids reach gut microbiota in the large intestine, where they are converted to phenylacetylglutamine (PAG), hippuric acid, and p-cresol sulfate, respectively. These gut-microbiota-derived amino acid derivatives are absorbed by the host and ultimately enter the systemic circulation where they may alter physiological and metabolic processes in the host.

FIG. 12. Forest plot displaying odds associated with lethal Prostate Cancer by quartile of trimethylamine-associated analytes.

FIG. 13. Forest plot displaying odds associated with Lethal Prostate Cancer by quartile of gut-microbiome-derived amino acid metabolites.

FIG. 14. Standard curves for LC/ESI/MS/MS analysis of TMAO, choline, betaine, carnitine, γ-butyrobetaine (butyrobet), crotonobetaine (crotonobet), hippuric acid (Hippuric), p-cresol sulfate (PCS) and phenylacetylglutamine (PAG). Varying levels of the indicated metabolites were mixed with 4 volumes of cold methanol containing their respective isotope labeled internal standards. 0.2 ul was injected onto LC/MS. Analyses were performed using electrospray ionization in positive-ion mode except for PCS in negative mode with multiple reaction monitoring of parent and characteristic daughter ions and retention time specific for components monitored. The transitions monitored were mass-to-charge ratio as described in Methods. Standard curve of each metabolite was generated by plotting peak area ratio to its corresponding isotope labeled standard versus concentration (in uM).

### DEFINITIONS

As used herein, the terms “cardiovascular disease” (CVD) or “cardiovascular disorder” are terms used to classify numerous conditions affecting the heart, heart valves, and vasculature (e.g., veins and arteries) of the body and encompasses diseases and conditions including, but not limited to arteriosclerosis, atherosclerosis, myocardial infarction, acute coronary syndrome, angina, congestive heart failure, aortic aneurysm, aortic dissection, iliac or femoral aneurysm, pulmonary embolism, primary hypertension, atrial fibrillation, stroke, transient ischemic attack, systolic dysfunction, diastolic dysfunction, myocarditis, atrial tachycardia, ventricular fibrillation, endocarditis, arteriopathy, vasculitis, atherosclerotic plaque, vulnerable plaque, acute coronary syndrome, acute ischemic attack, sudden cardiac death, peripheral vascular disease, coronary artery disease (CAD), peripheral artery disease (PAD), and cerebrovascular disease.

As used herein, the term “atherosclerotic cardiovascular disease” or “disorder” refers to a subset of cardiovascular disease that include atherosclerosis as a component or precursor to the particular type of cardiovascular disease and includes, without limitation, CAD, PAD, cerebrovascular disease. Atherosclerosis is a chronic inflammatory response that occurs in the walls of arterial blood vessels. It involves the formation of atheromatous plaques that can lead to narrowing (“stenosis”) of the artery, and can eventually lead to partial or complete closure of the arterial opening and/or plaque ruptures. Thus atherosclerotic diseases or disorders include the consequences of atheromatous plaque formation and rupture including, without limitation, stenosis or narrowing of arteries, heart failure, aneurysm formation including aortic aneurysm, aortic dissection, and ischemic events such as myocardial infarction and stroke

The terms “individual,” “host,” “subject,” and “patient” are used interchangeably herein, and generally refer to a mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos. In some embodiments, the subject is specifically a human subject.

As used herein “heart failure” refers to when the heart is unable to pump sufficiently to maintain blood flow to meet the body's needs. Signs and symptoms of heart failure commonly include shortness of breath, excessive tiredness, and leg swelling. The shortness of breath is usually worse with exercise, while lying down, and may wake the person at night. A limited ability to exercise is also a common feature. Common causes of heart failure include coronary artery disease including a previous or current myocardial infarction (heart attack), high blood pressure, atrial fibrillation, valvular heart disease, excess alcohol use, infection, and cardiomyopathy of an unknown cause.

As used herein “prostate cancer” refers to cancer of the prostate. The prostate is a gland in the male reproductive system that surrounds the urethra just below the bladder. Most prostate cancers are slow growing. Cancerous cells may spread to other areas of the body, particularly the bones and lymph nodes. It may initially cause no symptoms. In later stages, symptoms include pain or difficulty urinating, blood in the urine, or pain in the pelvis or back. Other late symptoms include fatigue, due to low levels of red blood cells. “Lethal prostate cancer” is prostate cancer that leads to death and Helgstrand et al., Eur J Cancer. 2017 October; 84:18-26 (herein incorporated by reference) provides diagnostic characteristics.

### DETAILED DESCRIPTION OF THE INVENTION

The present invention relates to systems, kits, and methods for identifying subjects with increased levels of phenylacetyl glutamine (PAG) or the combination of PAG and trimethylamine-n-oxide (TMAO) and/or N6-trimethyl-lysine (TML), and/or PSA, and/or betaine, and/or choline, as well as methods of determining risk of disease (e.g., CVD, heart failure, asthma, diabetes, thrombosis, and prostate cancer) based on such levels. In certain embodiments, the subjects are free of chronic kidney disease and/or have type II diabetes. In particular embodiments, subjects are treated with a therapeutic, such as a beta-adrenergic blocking agent, an alpha 2 adrenergic receptor agonist, an alpha 2 adrenergic receptor antagonist, an antibiotic or antibiotic cocktail, or a prostate cancer therapeutic. In certain embodiments, the subject is treated with a procedure such as brachytherapy, radiation therapy, or prostatectomy.

Work conducted during development of embodiments herein identified phenylacetylglutamine (PAG or PAGln) as strikingly associated with cardiovascular disease (CVD) and major adverse cardiovascular events (myocardial infarction, stroke or death). Gut microbiota suppression with antibiotics in both humans and mice depleted systemic levels of PAGln, and physiological levels of PAGln enhanced whole blood platelet adhesion and activation on collagen surfaces, platelet stimulus-dependent Ca2+ release, platelet hyper-responsiveness to multiple agonists, and both thrombus rate of formation and occlusion time in murine models of arterial injury.

It is noted that N6-trimethy-lysine, is also known as (S)-2-amino-6-(trimethylammonio)Hexanoate; (S)-2-amino-6-(trimethylammonio)Hexanoic acid; delta-Trimethyllysine; epsilon-N-Trimethyl-L-lysine; epsilon-Trimethyl-L-lysine; N(6),N(6),N(6)-Trimethyl-L-lysine; epsilon-N-Trimethyl-R-lysine; epsilon-Trimethyl-R-lysine; N(6),N(6),N(6)-Trimethyl-R-lysine; epsilon-N-Trimethyl-lysine; epsilon-Trimethyl-lysine; N(6),N(6),N(6)-Trimethyl-lysine; (S)-5-amino-5-Carboxy-N,N,N-trimethyl-1-pentanaminium; Trimethyllysine; 6-N-Trimethyl-L-lysine; 6-N-Trimethyl-R-lysine; 6-N-Trimethyl-lysine; epsilon-N-Trimethyl-lysine; TRIMETHYLLYSINE; Trimethyllysine hydroxide, inner salt, (S)-isomer; Trimethyllysine, (+−)-isomer; Trimethyllysine hydroxide, inner salt, (+−)-isomer; Trimethyllysine chloride, (S)-isomer; and Trimethyllysine chloride, (R)-isomer.

In certain embodiments, the antibiotics employed herein include, but are not limited to, Ampicillin; Bacampicillin; Carbenicillin Indanyl; Mezlocillin; Piperacillin; Ticarcillin; Amoxicillin-Clavulanic Acid; Ampicillin-Sulbactam; Benzylpenicillin; Cloxacillin; Dicloxacillin; Methicillin; Oxacillin; Penicillin G; Penicillin V; Piperacillin Tazobactam; Ticarcillin Clavulanic Acid; Nafcillin; Cephalosporin I Generation; Cefadroxil; Cefazolin; Cephalexin; Cephalothin; Cephapirin; Cephradine; Cefaclor; Cefamandol; Cefonicid; Cefotetan; Cefoxitin; Cefprozil; Cefhnetazole; Cefuroxime; Loracarbef; Cefdinir; Ceftibuten; Cefoperazone; Cefixime; Cefotaxime; Cefpodoxime proxetil; Ceftazidime; Ceftizoxime; Ceftriaxone; Cefepime; Azithromycin; Clarithromycin; Clindamycin; Dirithromycin; Erythromycin; Lincomycin; Troleandomycin; Cinoxacin; Ciprofloxacin; Enoxacin; Gatifloxacin; Grepafloxacin; Levofloxacin; Lomefloxacin; Moxifloxacin; Nalidixic acid; Norfloxacin; Ofloxacin; Sparfloxacin; Trovafloxacin; Oxolinic acid; Gemifloxacin; Perfloxacin; Imipenem-Cilastatin Meropenem; Aztreonam; Amikacin; Gentamicin; Kanamycin; Neomycin; Netilmicin; Streptomycin; Tobramycin; Paromomycin; Teicoplanin; Vancomycin; Demeclocycline; Doxycycline; Methacycline; Minocycline; Oxytetracycline; Tetracycline; Chlortetracycline; Mafenide; Silver Sulfadiazine; Sulfacetamide; Sulfadiazine; Sulfamethoxazole; Sulfasalazine; Sulfisoxazole; Trimethoprim-Sulfamethoxazole; Sulfamethizole; Rifabutin; Rifampin; Rifapentine; Linezolid; Streptogramins; Quinopristin Dalfopristin; Bacitracin; Chloramphenicol; Fosfomycin; Isoniazid; Methenamine; Metronidazol; Mupirocin; Nitrofurantoin; Nitrofurazone; Novobiocin; Polymyxin; Spectinomycin; Trimethoprim; Colistin; Cycloserine; Capreomycin; Ethionamide; Pyrazinamide; Para-aminosalicyclic acid; and Erythromycin ethylsuccinate.

In certain embodiments, the prostate cancer therapeutic is selected from the group consisting of: Abiraterone Acetate, Apalutamide, Bicalutamide, Cabazitaxel, Casodex (Bicalutamide), Darolutamide, Degarelix, Docetaxel, Eligard (Leuprolide Acetate), Enzalutamide, Erleada (Apalutamide), Firmagon (Degarelix), Flutamide, Goserelin Acetate, Jevtana (Cabazitaxel), Leuprolide Acetate, Lupron Depot (Leuprolide Acetate), Lynparza (Olaparib), Mitoxantrone Hydrochloride, Nilandron (Nilutamide), Nilutamide, Nubeqa (Darolutamide), Olaparib, Orgovyx (Relugolix), Provenge (Sipuleucel-T), Radium 223 Dichloride, Relugolix, Rubraca (Rucaparib Camsylate), Rucaparib Camsylate, Sipuleucel-T Taxotere (Docetaxel), Xofigo (Radium 223 Dichloride), Xtandi (Enzalutamide) Yonsa (Abiraterone Acetate), Zoladex (Goserelin Acetate), and Zytiga (Abiraterone Acetate).

### EXAMPLES

The following examples are for purposes of illustration only and are not intended to limit the scope of the claims.

### Example 1

### Detection of Phenylacetylglutamine

This Example describes how a metabolite (m/z=265.1188) identified as phenylacetylglutamine (PAG or PAGln) was discovered, and then shown in an independent cohort (n=4,000 subjects) to be strikingly associated with cardiovascular disease (CVD) and major adverse cardiovascular events (myocardial infarction, stroke or death). Gut microbiota suppression with antibiotics in both humans and mice depleted systemic levels of PAGln, and physiological levels of PAGln enhanced whole blood platelet adhesion and activation on collagen surfaces, platelet stimulus-dependent Ca2+ release, platelet hyper-responsiveness to multiple agonists, and both thrombus rate of formation and occlusion time in murine models of arterial injury. Functional and genetic engineering studies with human commensals, coupled with microbial colonization of germ-free mice, showed the microbial porA gene can facilitate dietary phenylalanine conversion into phenylacetic acid, with subsequent host generation of PAGln and phenylacetylglycine (PAGly) fostering heightened platelet responsiveness and in vivo thrombosis potential. Gain- and loss of-function studies employing genetic and pharmacological tools reveal PAGln mediates cellular events through G-protein coupled receptors (GPCRs), including α2A, α2B and β2-adrenergic receptors (ADRs). Collectively, these results reveal a therapeutic target for CVD—a metaorganismal pathway involving dietary phenylalanine and gut microbiota-dependent conversion into PAGln, which signals via adrenergic receptors.

### Materials and Methods

### Human Subjects

Clinical study population. Plasma samples and associated clinical data were collected as part of 2 studies at a tertiary care center. All subjects gave written informed consent and the Institutional Review Board of the Cleveland Clinic approved all study protocols. Metabolomics studies were performed on GeneBank samples, a large (n>10,000) well-characterized and longitudinal tissue repository with associated clinical database. It comprised sequential participants enrolled in the study GeneBank, which is composed of sequential stable subjects without evidence of acute coronary syndrome (cardiac troponin 1<0.03 ng/mL) who underwent elective diagnostic coronary angiography (cardiac catheterization or coronary computed tomography) for evaluation of CAD (Tang et al., 2013; Wang et al., 2011; Wang et al., 2007). All subjects had extensive clinical and longitudinal outcome data monitored, including adjudicated outcomes over the ensuing 3 to 5 years for all participants after enrollment. MACE was defined as death, nonfatal myocardial infarction, or nonfatal cerebrovascular accident (stroke) following enrollment. Discovery cohort (n=1,157) for untargeted metabolomics and independent validation cohort (n=4,000) were derived from sequential consenting subjects enrolled in GeneBank. Fasting high-sensitivity C-reactive protein, and lipid profiles were measured on the Roche Cobas platform (Roche Diagnostics).

Normal ranges samples were collected from non-fasting subjects undergoing community health screens. From these, a random subset (n=25) of subjects were selected who had no medical history of cardiovascular or metabolic diseases, normal vital signs and screening laboratory results (lipid profile, basic chemistry panel), and no history of taking any medications.

In additional studies, healthy volunteers (n=10) were consented and subjected to cocktail of antibiotics for 7 days (metronidazole (500 mg twice daily) plus ciprofloxacin (500 mg once daily); there are other 2 combinations: ciprofloxacin, metronidazole, and vancomycin; and ciprofloxacin, metronidazole, vancomycin, and neomycin). Volunteers participating were excluded if they were pregnant, had chronic illness (including a known history of heart failure, renal failure, pulmonary disease, gastrointestinal disorders, or hematologic diseases). Blood was collected after overnight fasting (n=15 total number of samples) at 3 different time points: base line (before antibiotics treatment (pre-Abx): right after the seven day antibiotics regiment (Abx) and after antibiotics washout to permit repopulation of gut microorganisms (Post-Abx; min three weeks). Suppression of gut microbial metabolite levels were performed under approved protocol registered under ClinicalTrials.gov Identifier: NCT01731236.

### Untargeted LC-MS/MS Analysis of Human Plasma Samples

Untargeted analysis of plasma samples using HILIC-MS. Untargeted analysis of plasma samples was similar to that described previously (Tsugawa et al., 2015). In brief, extraction of polar metabolites from a plasma sample (30 μL) was carried out using an acetonitrile/isopropanol/water (3:3:2, v/v/v) mixture (0 mL). Aliquots (300 μL) were evaporated, resuspended using an acetonitrile/water (4:1, v/v) mixture (60 μL) containing series of deuterated internal standards, vortexed, centrifuged, and 50 μL was transferred to a glass vial with a microinsert. Hydrophilic interaction chromatography (HILIC) analysis was performed on a system including an Agilent 1290 Infinity LC system (Agilent Technologies) with a pump (G4220A), a column oven (G1316C), an autosampler (G4226A), and a TripleTOF 5600+(SCIEX). Extracts were separated on an Acquity UPLC BEH Amide column (150×2.1 mm; 1.7 μm. Waters) coupled to a Waters Acquity UPLC BEH Amide VanGuard precolumn (5×2.1 mm; 1.7 μm). The column was maintained at 45° C. at a flow rate of 0.4 mL/min. The mobile phases consisted of (A) water with ammonium formate (10 mM) and formic acid (0.125%) and (B) 95:5 (v/v) acetonitrile/water with ammonium formate (10 mM) and formic acid (0.125%). The separation was conducted under the following gradient: 0 min 100% B; 0-2 min 100% B: 2-7.7 min 70% B; 7.7-9.5 min 40% B; 9.5-10.25 min 30% B; 10.25-12.75 min 100% B: 12.75-17.75 min 100% B. A sample volume of 1 μl was used for the injection. Sample temperature was maintained at 4° C. The QTOFMS instrument was operated in electrospray ionization in positive ion mode with the following parameters: curtain gas, 35 psi; ion source gas 1, 50 psi; ion source gas 2, 50 psi; temperature, 300° C.: ion spray voltage floating, 4.5 kV: declustering potential, 100 V; acquisition speed, 2 spectra/s. For data processing MZmine 2 (37) and MultiQuant (SCIEX) software programs were used.

### Compound Identification

To chemically define the structures of the plasma analyte with m/z 265.1188, PAGln in plasma was identified by HPLC/high-resolution mass spectrometer with the same retention time, high-resolution mass, and fragmented ions as standard. Plasma supernatant after methanol precipitation of protein was analyzed by injection onto a C18 column or HILIC column using a 2 LC-20AD Shimadazu pump system with SIL-HTC autosampler interfaced (Shimadzu Scientific Instruments, Inc., Columbia, Md., USA) in tandem with a TripleTOF 5600 mass spectrometer (AB SCIEX). Separation was performed by employing the following gradients: For reverse phase chromatographic Kinetex c18 column (50 mm 2.1 mm; 2.6 μm) (Cat #00B-4462-AN, Phenomenex, Torrance, Calif.) was used. Solvent A (0.1% acetic acid in water) and B (0.1% acetic acid in acetonitrile) were run under the following gradient: 0.0 min (0% B); 0.0-2.0 min (0% B); 2.0-5.0 min (0%→B420% B); 5.0-6.0 min (20%→B460% B); 6.0-7.5 min (60%→B470% B); 7.5-8.0 min (70% B→100% B); 8.0-9.5 min (100%); 9.5-10 min (100%→B40% B); 10.0-15.0 min (0% B) with flow rate of 0.4 mL/min and the injection volume of 1 μL. For separation on HILIC column BEH amide column (Bridge column; BEH amide 2.1×150 mm×2.5 μm column) (Cat#186006724. Waters, Ireland) was used. Solvent A (10 mM ammonium acetate+0.125% acetic acid in water) and B (10 mM ammonium acetate+0.125% acetic acid in 95% acetonitrile and 5% water) were run under the following gradient: 0.0 min (100%); 0.0-0.5 min (100% B470% B); 0.5-1.0 min (70% B→60% B); 1.0-2.0 min (60% B); 2.0-7.0 min (60% B→50% B); 7.0-8.0 min (50% B); 8.0-9.0 min (50% B→100% B); 9.0-14.0 min (100%). The flow rate was 0.2 mL/min from 0.0-9.0 min and 0.4 mL/min 9.5-14 min.

To further confirm the compound structure, compound with m/z 265.1188 was partially purified from human plasma with the following procedures. First proteins from human plasma (500 mL) were precipitated with ice-cold methanol (1:4; v/v), and supernatant was dried under the reduced pressure. The dry residue was dissolved in water with 0.1% acetic acid (50 mL) and subjected to sold phase extraction on C18 cartages (Strata C18_E (55 μm, 70 A; 20 g/60 mL Giga Tubes, Phenomenex, Cat#8B-S001-VFF). Eluted fraction were analyzed by mass spec and fractions containing compound with m/z 265.1188 were pulled and dried under the reduced pressure. The dried reside was further purified on a semi-preparative C18 column (ODS, 10×250 mm; 5 μm) (Beckman Coulter) by HPLC. The mobile phases consisted of solvents A (0.2% acetic acid in water) and B (0.2% acetic acid in 90% methanol in water) under the following gradient: 0-23 min (0% B→80% B): 23-37 min (80% B→100% B); 27-32 min (100% B). Fractions were collected every 0.5 min. Fractions containing compound with m/z 265.1188 were pulled and dried under the reduced pressure. Dry residue was dissolved in water solution of NaOH (5.0 mL, 5 mM). An aliquot (300 μL) of sample supernatant was transferred to a glass tube and mixture of propanol/pyridine (3:2, v/v, 300 μL) and propyl chloroformate (50 μL) was added to the samples, and the tube were capped, vortexed (1 min), and sonicated (2 min). Hexane (300 μL) was added, and liquid/liquid extraction was performed by vortexing the samples for 5 min. The samples were centrifuged (20 min, 5000 g), and the organic layer was transferred to another glass tube and dried under stream of nitrogen. Dry residue was dissolved in 50% methanol in water and subjected to LC-MS analysis on C18 column as described above.

### Targeted LC-MS/MS Analysis of Human Plasma Samples

Stable-isotope-dilution LC-MS/MS was used for quantification of PAGln in human (20 μL) plasma. Ice cold methanol containing internal standard (D₅-phenylacetylglutamine was added to the plasma samples (80 μL), followed by vortexing and centrifuging (14,000 rpm; 4° C. for 15 min). The clear supernatant was transferred to a to glass vials with microinserts and submitted to LC-MS/MS analysis. LC-MS/MS analysis was performed on a chromatographic system composed of two Shimadzu LC-30 AD pumps (Nexera X2), a CTO 20AC oven operating at 30° C., and a SIL-30 AC-MP autosampler in tandem with triple quadruple mass spectrometer from 8050 series (Shimadzu Scientific Instruments. Inc., Columbia, Md., USA). For chromatographic separation Kinetex C18 column (50 mm×2.1 mm: 2.6 μm) (Cat # 00B-4462-AN, Phenomenex, Torrance, Calif.) was used. Solvent A (0.1% acetic acid in water) and B (0.1% acetic acid in acetonitrile) were run the following gradient: 0.0 min(0% B); 0.0-2.0 min (0% B); 2.0-5.0 min (0% B→20% B); 5.0-6.0 min (20% B→60% B); 6.0-7.5 min (60% B→70% B): 7.5-8.0 min (70% B→100% B); 8.0-9.5 min (100%): 9.5-10 min (100% B→0% B); 10.0-15.0 min (0% B) with flow rate of 0.4 mL/min and the injection volume of 1 μL. In the ion spray positive mode with multiple reaction monitoring (MRM) was used for the detection. Following transitions were used: m/z 265.2→130.15 for PAGln and D5-PAGln m/z 270.1→130.15. The following ion source parameters were applied: nebulizing gas flow, 3 l/min; heating gas flow, 10 l/min: interface temperature, 300° C.; desolvation line temperature, 250° C.: heat block temperature, 400° C.: and drying gas flow, 10 l/min. Limit of detection (LOD) and limit of quantification (LOQ) were 0.010 and 0.033 μM, respectively. Quality control samples were run with each batch of samples and inter-batch variations expressed as coefficient of variation (CV) were less than 10%.

In Vitro Screening of Phenylacetic and Phenylpropionic Acid Production from Phenylalanine by C. sporogenes Mutants

For creating C. sporogenes double knock out mutants, C sporogenes lacking cutC gene was made by a new CRISPR-Cas9-based genetic system for Clostridium, as previously described (Guo et al., 2018). A second gene was disrupted using the Clostron mutagenesis system as previously described (Dodd et al., 1017). Briefly, the porA or fldH retargeted pMTL007C-E2 vector was conjugated into C. sporogenes ΔcutC and thiamphenicol resistant colonies were selected. After verifying by diagnostic PCR to contain the plasmid of interest, these colonies were spread onto TYG plates supplemented with erythromycin. To assess the second gene disruption, diagnostic PCR was performed using a genomic DNA isolated from the erythromycin resistant colony as a template. The primer set was designed to identify the insertion of intron into the target gene.

C sporogenes mutants were grown on tryptic soy blood agar plates (TSBA; Anaerobe Systems, Cat# AS-542) anaerobically for 48 to 72 h at 37° C. Single colonies were then inoculated into Mega Medium (3 mL) and grown anaerobically overnight at 37° C. Overnight culture (100 μL) was added to fresh mega media (1 mL) containing ¹³C₆-phenylalanine (100 μM) and cultured an additional 24 h. Cultured media was centrifuged and supernatant was pass through 3K centrifugal filers (Amicon Ultra-0.5 mL Centrifuge filters. Ultracel-3K, Merck Millipore Ltd.). Filtrate (20 μL) was subjected to stable-isotope-dilution LC-MS/MS analysis for quantification of ¹³C₈-phenylacetic acid and ¹³C₉-phenylpropionic acid. Ice cold methanol containing internal standard (¹³C₂-phenylacetic acid: 80 μL) was added to the supernatant, followed by vortexing and centrifuging (14,000 rpm; 4° C. for 15 min). The clear supernatant was transferred to a glass vial with microinserts and submitted to LC-MS/MS analysis. LC-MS/MS analysis was performed on a chromatographic system composed of two Shimadzu LC-30 AD pumps (Nexera X2), a CTO 20AC oven operating at 30° C., and a SIL-30 AC-MP autosampler in tandem with triple quadruple mass spectrometer from 8050 series (Shimadzu Scientific Instruments, Inc., Columbia, Md., USA). For chromatographic separation Kinetex C18 column (50 mm×2.1 mm; 2.6 μm) (Cat # 00B-4462-AN, Phenomenex, Torrance, Calif.) was used. Solvent A (0.1% acetic acid in water) and B (0.1% acetic acid in acetonitrile) were run the following gradient: 0.0 min(0% B): 0.0-2.0 min (0% B): 2.0-5.0 min (0% B→20% B): 5.0-6.0 min (20% B→60% B): 6.0-7.5 min (60% B→70% B); 7.5-8.0 min (70% B→100% B); 8.0-9.5 min (10(Wo); 9.5-10 min (100% B→0% B): 10.0-15.0 min (0% B) with flow rate of 0.4 mL/min and the injection volume of 1 μL. In the ion spray negative mode with multiple reaction monitoring (MRM) was used for the detection. Following transitions were used: m/z 135.0→91.0 for phenylacetic acid; 143.0→98.0 for ¹³C₈-phenylacetic acid; 149.50→105 and 158→113.0 for phenylpropionic and ¹³C₉-phenylpropionic acid, respectively and 137.0→92.0 for ¹³C₂-phenylacetic acid (internal standard).

### Murine Antibiotic Challenge

All animal studies were under protocols approved by the Cleveland Clinic Institutional Animal Care and Use Committee. Whole blood was collected via saphenous vein (survival collection) into heparin treated capillaries. C57BL/6J males (n=l 1) and females (n=15) 8-10 weeks old were treated with an antibiotic cocktail to suppress gut-microbial growth as previously described (Devlin et al., 2016). Briefly, mice were on regular chow diet and blood for base line measurements were collected. After that the mice were given an antibiotic cocktail in their drinking water consisting of kanamycin (0.4 mg/mL), gentamycin (0.035 mg/mL), colistin (0.057 mg/mL), metronidazole (0.215 mg/mL), vancomycin (0.045 mg/mL), and erythronmcin (0.01 mg/mL). The antibiotic cocktail was administered for a total of 3 days, and then second blood collection was done. After that mice were put back on regular water without antibiotics along with addition into cages fecal pellets from conventionally reared littermates never treated with antibiotics to permit repopulation of gut microorganisms. Third blood collection was done 7 days after the antibiotics withhold.

### Whole Blood In Vitro Thrombosis Assay

Microfluidics experiments were performed using the Cellix Microfluidics System (Cellix Ltd., Dublin, Ireland). Where indicated, each micro channel of a Vena8 Fluoro+ biochip was coated with type 1 collagen (15 μL; 150 μg/mL) and the biochip was then place in a humidified box overnight at 4° C. Each channel of the Vena8Fluoro+ biochip was washed with 1×PBS using the Mirus Nanopump before placing the biochip on the microscope. Images were collected using an HC Plan Apo 20X/0.7NA lens on a Leica DMI6000 inverted microscope equipped with an environmental chamber and a Hamamatsu ImagEM cooled CCD camera. Whole blood collected from consented healthy volunteers was fluorescently tagged with Calcein AM and was pretreated with PAGln (100 μM final, pH 7.4) or normal saline control for 30 min at 22° C. After the incubation, blood was then perfused over chips coated with or without immobilized type 1 collagen (150 μg/mL) at physiological shear rate (67.5 dynes/cm²) using a multi-channel microfluidic device for 3 minutes. Images of fluorescent platelets adhering to the collagen coating were captured every 5 seconds during that time. At the end of the experiment, the tube containing the platelets was removed and the 1×PBS in the biochip reservoir was drawn through the channel at 20 dynes. Ten images were captured along the length of the channel during that time. Platelet activation and adherence to the collagen surface was then quantified with computer assisted tomographic analyses as previously described (Gupta et al., 2015; Srikanthan et al., 2014).

### Platelet Preparation and Aggregometry Assays in Humans

The aggregometry assays were performed as previously described (Zhu et al., 2016). Briefly, the whole blood was collected from consenting healthy donors using sodium citrate (0.109 M) as anticoagulant. Platelet rich plasma (PRP) was separated by centrifuging at 100×g for 10 min at 22° C. Platelet poor plasma (PPP) was prepared by further centrifugation at I 1,000×g for 2 min. Platelets were counted using a hemocytometer and for aggregometry assays, concentrations were adjusted to 2×108/mL with PPP. To prepare washed platelets for studies, prostaglandin E1 (PGE-1; 100 nM) was added to PRP and the PRP was then centrifuged at 500×g for 20 min at 22° C. The platelet pellet was gently washed with a modified phosphate buffer saline (NaCl (137 mM), KCl (2.7 mM), Na₂HPO₄ (12 mM), MgCl₂ (1 mM), and glucose (5.5 mM); pH 7.4) with PGE-1 (100 nM), and centrifuged again at 500×g for 20 min. Platelet pellets were then re-suspended in modified Tyrode's buffer (NaCl (137 mM), KCl (2.7 mM), NaHCO₃ (12 mM), NaH₂PO₄ (0.4 mM), HEPES (5 mM), glucose (5.6 mM), BSA (0.35%): pH 7.4). ADP (up to 4 μM), TRAP6 (up to 10 μM) and collagen (up to 1.0 μg/mL) were used as indicated to initiate aggregation with constant stirring (600 rpm). Platelets were pre-incubated with PAGln or PAGly (100 μM final or the indicated concentration) for 30 minutes at 22° C. before platelet aggregation was performed.

### Intracellular Ca²⁺ Measurements

To prepare washed platelets for intracellular Ca²⁺ measurements, 100 nM prostaglandin E1 (PGE-1) was added to PRP and the PRP was then centrifuged at 500×g for 20 min at 22° C. The platelet pellet was gently washed with a modified phosphate buffer saline (NaCl (137 mM), KCl (2.7 mM), Na₂HPO₄ (12 mM), MgCl₂ (1 mM), and glucose (5.5 mM), pH 7.4) with PGE-1 (100 nM), and centrifuged again at 500×g for 20 min. Platelet pellet was then re-suspended in modified Hank's buffered salt solution (HBSS-BSA-glucose; NaCl (0.137 M). KCl (5.4 mM), Na₂HPO₄ (0.25 mM KH₂PO₄ ( ), 0.44 mM), CaCl₂ (1.3 mM), MgSO₄ (1.0 mM), NaHCO₃ (4.2 mM), glucose (5 mM) and BSA (0.1%)) with 100 nM PGE-1. For ratiometric fluorescence measurements, washed platelets were incubated with Fura 2-AM (1 μM) at 22° C. for 30 min. Excess Fura 2-AM was removed by centrifugation at 500×g for 30 min. Platelet pellets were then re-suspended in modified Hank's buffered salt solution and changes in [Ca²⁺], was monitored by measuring Fura 2-AM fluorescence using 340/380 nm dual-wavelength excitation and an emission of 510 nm at 37° C. with constant stirring in a temperature controlled spectrofluorometer (Zhu et al., 2016).

### Gnotobiotic Mouse Colonization

All experiments involving mice were performed using protocols approved by the Cleveland Clinic Animal Care and Use Committee. C57BL/6 female mice aged 8-10 weeks were maintained as a colony at the University of Wisconsin-Madison or University of Nebraska-Lincoln gnotobiotic animal facilities in a controlled environment in plastic flexible film gnotobiotic isolators under a strict 14 h light/10 h dark cycle and received sterilized water and food ad libitum. Animals were shipped germ free to the Cleveland Clinic Lemer Research Institute Gnotobiotic facility. After arrival mice were housed in sealed sterilized cages with HEPA filters.

C. sporogenes mutants were grown on tryptic soy blood agar plates (TSBA; Anaerobe Systems, Cat# AS-542) anaerobically for 48 to 72 h at 37° C. Single colonies were picked and used to inoculate Mega Medium (3 mL) in prepared Hungate Tubes. Cultures were grown anaerobically for 18-24 h at 37° C. At that time an aliquot of culture (500 μL) was removed and the remaining bacterial culture was diluted 1:1 with glycerol (40%) in water (v:v) and store at −80° C. The aliquot was centrifuged and supernatant screened on the LC/MS for metabolite profile while DNA was isolated from the pellet for both 16s rRNA gene sequencing (described below) and PCR confirmation of presence/absence of porA and fldH. Germ-free, C57Bl/6, male, 8-10-week-old mice were mono-colonized by oral gavage with ˜0.2 mL of bacterial culture inside the biological safety cabinet, using indicated C. sporogenes mutants. Mice were maintained on a sterilized diet and 24 h prior to in vivo thrombosis were injected with filter sterilized folic acid (250 mg/kg). At the time of sacrifice (2-7 days post colonization), mice were subjected to carotid artery FeCl₃ injury thrombosis assay and tissues were collected immediately after the assay, frozen, and stored at −80° C. Following colonization, the investigator was not blinded from treatment groups to avoid cross contamination.

To confirm colonization DNA was isolated from flash frozen cecal contents of colonized mice with the NucleoSpin Tissue kit (Macherey-Nagel, Cat.#740952.250) according to manufactures instructions for bacterial DNA isolation. Isolated DNA was used in a PCR reaction with GoTaq Green Master Mix and the 8F and 1492R 16S rRNA universal primers. PCR reactions were carried out in a 96 well plate with 20 μl final volume as follows: 95° C. for 2 minutes: 95° C. for 30 seconds, 51° C. for 30 seconds, 72° C. for 1 minute and 20 seconds (×30); 72° C. for 5 minutes. Completed reactions were sent to Eurofins Genomics for PCR cleanup and 16S rRNA gene sequencing using their standard house primers (16F). Sequence identity was confirmed using NCBI BLAST.

### Carotid Artery FeCl₃ Injury Thrombosis Assay

Mice post IP injection of vehicle (normal saline), PAGln (50 mg/kg), PAGly (50 mg/kg) and germ free 48 h post colonization were anesthetized and subjected to a common carotid artery injury as previously described (Zhu et al., 2016). Briefly, rhodamine 6G (100 μL; 0.5 mg/mL) was injected directly into the right jugular vein to label platelets. The left carotid artery was exposed and injured by placing a FeCl₃-soaked filter paper for 1 minute. Thrombus formation was observed in real time using intravital fluorescence microscopy equipped with video recording. Time to cessation of blood flow through clot formation for all studies was determined by visual inspection by two different investigators. End points were set as cessation of blood flow for 30 seconds to 30 minutes.

### Dynamic Mass Redistribution (DMR) Studies on Suspension Cells

The DMR experiments on suspension cells, MEG01 and HEL92.1.7 were performed by growing the cells in RPMI 1640 media supplemented with fetal bovine serum (FBS; 10%), penicillin (100 U/mL) and streptomycin (100 μg/mL) in a humidified atmosphere at 37° C. in 5% CO₂. Before proceeding for the experiments, cells were subjected to overnight incubation in reduced serum media opti-MEM. Thereafter, the cells were washed using 1×HBSS buffer with HEPES (20 mM; pH 7.4) and seeded into a 96-well fibronectin coated epic coming DMR plate (Cat. 5082-Corning) with a density of ˜80-100 k cells per well suspended in of the same buffer (100 μL). The cells were briefly centrifuged at room temperature at low speed (100 g for 10-15 sec) to allow settling down the cells at the bottom of the plate. After that, cells were allowed to temperature equilibrate for 1 h at room temperature prior to run DMR measurement. Firstly, basal DMR responses were graphed for around 15 min to obtain a baseline read that defines the zero level and also ensures the signal is stable. For the DMR dose response experiments, we added (25 μL; 5× concentration) of different increasing concentrations of the compound of interest (PAGln, norepinephrine (Norepi) and phenylalanine (Phe)) and read DMR signal for 60-90 minutes in a Corning Epic BT system (Product code 5053). The G-protein modulator DMR studies on the suspension cells was performed by incubating the cells with pertussis toxin (PTX; 100 ng/mL) (Cat. 3097-Tocris) for 30 min, cholera toxin (CTX; 1 μg/mL) (Cat. C9903-Sigma) for 45 min, YM-254890 (Cat. 257-00631-Wako; 0.5 μM) for 30 min and SCH-202676 (1 μM) (Cat. 1400-Tocris) for 30 min before addition of the compound of interest (PAGln (100 μM), norepinephrine (10 μM) and collagen (10 μg/mL)). The DMR studies with siRNAs on MEG01 was carried out by transfecting the cells with ADRA2A, ADRA2B, ADRB2 and negative control silencer select siRNAs (combination of 3 siRNAs for each gene) with a concentration of 1 μmol of each of the three siRNAs per well in 96-well DMR plates following RNAi transfection protocol (silencer select human GPCR siRNA library V4 protocol 2013—Ambion-Lipofactamine RNAiMAX-invitrogen, Cat. 13778150). The siRNA transfected cells were subjected to DMR reading after 40 h post transfection by treating them with PAGln (100 μM), epinephrine (10 μM) and ATP (50 μM). The DMR studies with adrenergic receptor (ADR) inhibitors on MEG01 were performed by treating the cells with ICI118,551 (10 μM), propranolol (10 μM) and RX821002 (10 μM) for 30 min before addition of the compound of interest (PAGln (100 μM), ISO (10 μM), B-HT933 (50 μM) and ATP (50 μM)). DMR response signal was recorded for 60-90 minutes post compound of interest addition.

### Dynamic Mass Redistribution (DMR) Studies on Adherent Cells

The DMR experiments in adherent cell lines was carried out on parental HEK293, α2B-HEK293 (stable) and β2-HEK293 (stable) cells by seeding them into EPIC-coming fibronectin coated 96-well DMR microplates (Cat. 5082-Corning) with a density of ˜50 k cells per well. The cells were grown in DMEM (100 μL) media supplemented with FBS (10%), penicillin (100 U/mL) and streptomycin (100 μg/mL) in a humidified atmosphere at 37° C. in 5% CO₂ for 1 day before proceeding for the DMR experiments. For gain of function studies of α2A ADR, parental HEK293 cells were transfected with pcDNA3.1(+)-N-DYK empty vector or with pcDNA3.1(+)-ADRA2A-N-DYK clone (clone id: OHu19050C-GenScript) using Lipofactamine 3000 Transfection kit-Invitrogen (Cat. L3000008). DMR studies were performed on transiently transfected cells after 2 days post transfection. Prior to run the DMR experiment, cells were washed with 1×HBSS buffer with HEPES (20 mM, pH 7.4) and allowed to temperature equilibrate for 1 h at room temperature in 100 μL of the same buffer. Basal DMR response were graphed for around 15 min to obtain a baseline read that defines the zero level and also ensures the signal is stable. The DMR signal was monitored after adding the compound of interest (PAGln (100 μM), ISO (10 μM), B-HT933 (50 μM) and ATP (50 μM)) for 60-90 minutes. For inhibitor studies ICI118,551 (10 μM), propranolol (10 μM) and RX821002 (10 μM) were incubated for 30 min before addition of compound of interest.

Real-time (RT) PCR in MEG01 cells: Total RNA was isolated from MEG01 cells following TRIZOL RNA isolation protocol post 40 h of siRNA transfection. Reverse Transcription was performed using High-Capacity RNA to cDNA kit (Applied Biosystem-Cat. no 4387406) following recommended protocol. Real time (RT) PCR was carried out following TaqMan Gene Expression Assays protocol using RT primers and probes of ADRA2A (assay Id Hs01099503_s1), ADRA2B (assay Id Hs00265081 s1) and ADRB2 (assay Id Hs00240532_s1) from ThermoFisher Scientific.
cAMP assay in MEG01, Platelets and HEK293 cells: Intracellular cAMP levels were measured using CatchPoint Cyclic-AMP Fluorescent Assay Kit from molecular devices (Cat. R8089). Briefly, MEG01 cells were washed with 1×HBSS buffer with HEPES (20 mM; pH 7.4) and re-suspended in stimulation buffer (1×HBSS, HEPES (20 mM); pH 7.4, IBMX (0.5 mM), Rolipram (0.1 mM) and BSA (0.1%)). The cells were further seeded into 96 well cell culture plates with a density of ˜100 k cells per well in 100 μL stimulation buffer. Similarly, human washed platelets re-suspended very gently in stimulation buffer and seeded into a 96-well cell culture plates with ˜4-6 million platelets per well in 100 μL stimulation buffer. Both the MEG01 cells and platelets were kept thereafter at 37° C. incubator for 10 min before addition of the inhibitors or test compounds. Following incubation with stimulation buffer, 10 μL (10× concentration) of modulators/inhibitors (when necessary) were added and kept for 15 min at 37° C. After that, 10 μL (10× concentration) of the test compounds (PAGln (100 μM) and ISO (10 μM)) were added and incubated for different time periods at 37° C. The test compounds were made in buffer T (1×HBSS, HEPES (20 mM); pH 7.4, IBMX (0.5 mM) and Rolipram (0.1 mM)). The reaction was stopped by adding 50 μL of lysis buffer (provided in the kit CatchPoint Cyclic-AMP Fluorescent Assay Kit Cat. R8089-Molecular Devices), further added with IBMX (0.5 mM) and Rolipram (0.1 mM)) and agitated for 10 min in a plate shaker to facilitate cell lysis. Lysed cells are immediately proceeded to cAMP assay. For the quantification of cAMP, the lysed sample (40 μL), buffer control and cAMP calibrators were added in appropriate wells of a 96-well clear bottom, black wall cAMP assay plates (provided in the kit). Thereafter, reconstituted rabbit anti-cAMP antibody (40 μL) was added to all the wells and mix for 5 min on a plate shaker to ensure mixing. In the next step, reconstituted HRP-cAMP conjugate (40 μL) was added to every well, mixed properly and kept at room temperature for 2 h. The plate contents were aspirated thereafter and washed with wash buffer (300 μL (provided in the kit)) 4 times. Spotlight red substrate (provided in the kit) was added to each well minimizing the time between starting and finishing in dark. The plate was covered with aluminum foil and left at room temperature for at least 10 min before reading fluorescence intensity in a FlexStation 3 Multi-Mode Microplate Reader-Molecular devices. For cAMP assay with G-protein modulator, pertussis toxin (PTX; 100 ng/mL) (Cat. 3097-Tocris) was incubated for 45 min, cholera toxin (CTX; 1 μg/mL) (Cat. C9903-Sigma) was incubated for 60 min, YM-254890 (0.5 μM) (Cat. 257-00631-Wako) was incubated for 45 min and SCH-202676 (1 μM) (Cat. 1400-Tocris) was incubated for 45 min by adding 10 μL (10× concentration) of each modulators per well before addition of the test compound. The cAMP assay with ADR inhibitors in MEG01 cells was performed by treating the cells with ICI118,551 (10 μM), propranolol (10 μM) and RX821002 (10 μM) by adding 10 μL (10× concentration) of the respective inhibitors per well for 15 min before addition of the compound of interest. For cAMP assay in adherent cell lines, parental HEK293, and β2-HEK293 (stable) cells were seeded into 96-well cell culture microplates with approximately 50 k cells per well in DMEM (100 μL) media supplemented with FBS (10%), penicillin (100 U/mL) and streptomycin (100 μg/mL) in a humidified atmosphere at 37° C. in 5% CO₂. Cells were grown for 24 h before proceeding for cAMP experiment following similar method as described for MEG01 cells.
Intracellular Ca²⁺ measurement: Intracellular Ca²⁺ in MEG01, HEL92.1.7, parental HEK293, β2-HEK293 (stable) cells, empty vector (EV) transfected HEK293 cells, ADRA2A transfected HEK293 cells and in α2B-HEK293 stable cells was measured using FLIPR Calcium 5 Assay Kit-Molecular devices (Cat. R8186). Briefly, ˜100 k cells per well for suspension cells or ˜50 k cells per well for adherent cells were seeded into 96 well clear bottom, black wall cell culture plates (Cat no. 353219 Falcon) in of assay buffer (100 μL; 1×HBSS, 20 mM HEPES, pH 7.4). Adherent cells were grown for 1 day prior to the experiment. Calcium assay reagent component A (provided in the kit) was re-suspended in Component B (10 mL; provided in the kit) and mixed by vortex for 1 to 2 min to prepare the loading buffer. Probenecid (100 μL of 250 mM) was added in the loading buffer before proceeding for the assay. Loading buffer (100 μL) was added to each well of the 96 well plate. The plates were incubated for 1 h at 37° C. and thereafter kept at room temperature until used for the assay. Test compound 96-well microplate was prepared adding 5× concentration of the compound of interest (PAGln (100 μM), ADP (10 μM) and TFLLR-NH₂ (10 μM)) in appropriate wells. At the time of compound addition, 50 μL of the test compound was added to the assay plates in respective wells in a FlexStation 3 Multi-Mode Microplate Reader-Molecular devices. The Ca²⁺ level was monitored in real time post compound addition as relative fluorescent unit (RFU). The maximum RFU peak minus minimum base line RFU was used as the net measurement of Ca²⁺ level.

### Statistics

Student's t test (2 tailed) or Wilcoxon's rank-sum test for continuous variables and χ2 test for categorical variables were used to examine the differences between groups. Rank-based nonparametric Kruskal-Wallis test was used for non-normally distributed data. Numerical data are presented as mean f SD or median (25th-75th percentile=Q1-Q3). Categorical data are presented as n (%). HR for MACE at 3-year follow-up and corresponding 95% CI were estimated using both univariable (unadjusted) and multivariable (adjusted) Cox models. Kaplan-Meier analysis with Cox proportional hazards regression was used for time-to-event analysis to determine HR and 95% 95% CI for MACE. Adjustments were made for individual traditional cardiac risk factors including age, sex, HDL, LDL, triglycerides, hyperlipidemia, smoking, diabetes mellitus, systolic blood pressure, and high-sensitivity C-reactive protein level. All analyses were performed using R 3.4.2 (Vienna, Austria, 2017). P values<0.05 were considered statistically significant.

### Results

Untargeted Metabolomics Identifies Phenylacetylglutamine (PAGln) is Associated with CVD

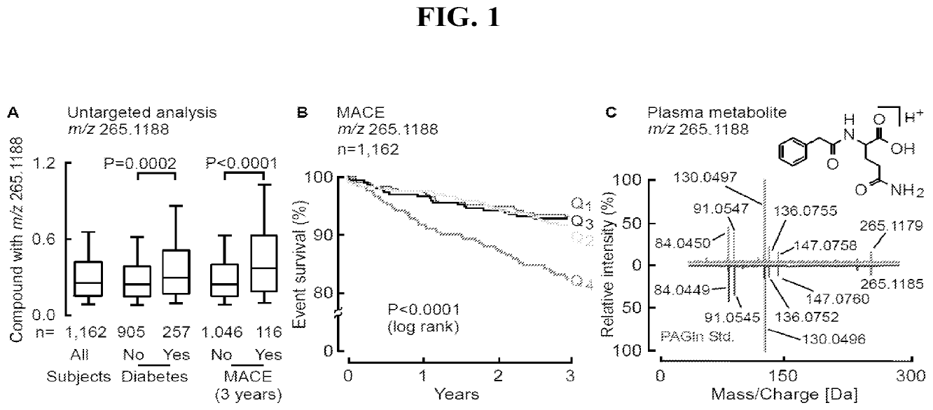

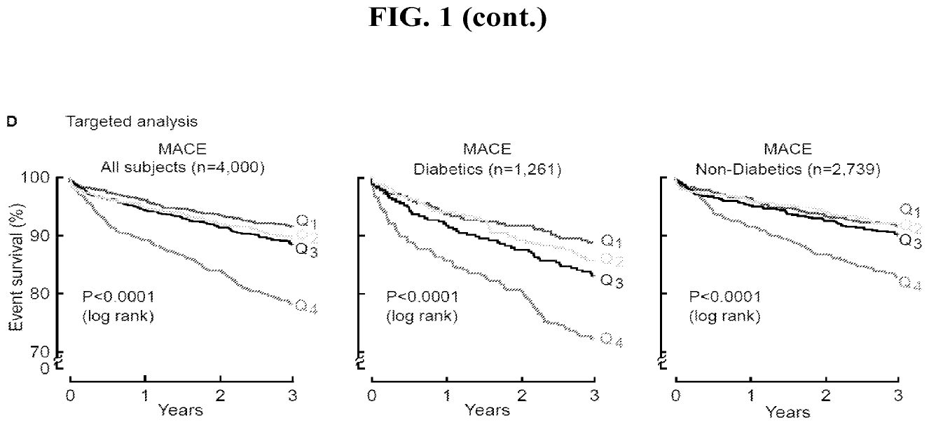

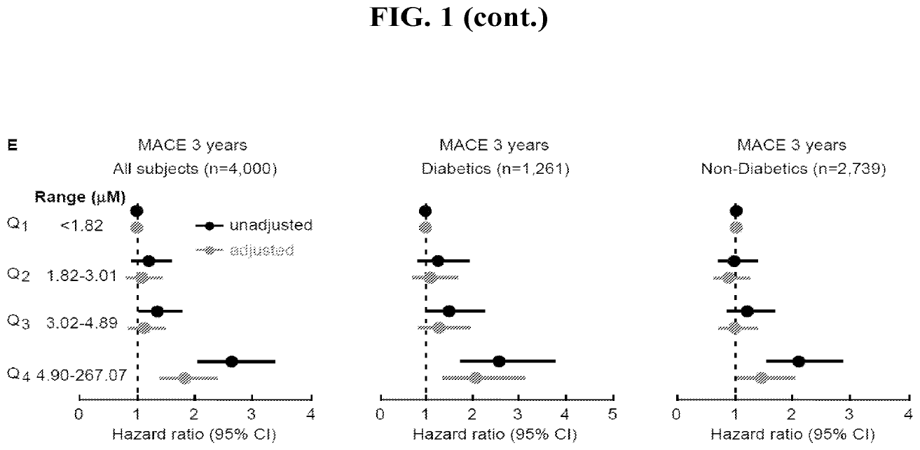

In an initial Discovery Cohort (n=1,162) untargeted metabolomics was performed on plasma from sequential stable subjects undergoing elective diagnostic cardiac evaluation with longitudinal (3 years) follow-up, as described under Methods. Plasma analytes were prioritized based on three screening inclusion criteria: (i) association with incident (3 year) major adverse cardiac events (MACE=myocardial infarction, stroke or death); (ii) the presence of T2DM; and (iii) poor correlation with indices of glycemic control (i.e. fasting glucose, HgbA1C and insulin/glucose). Of the spectral features in plasma monitored, the top MACE-associated predicted analytes were prioritized into two lists—those of known versus those of unknown structures at the time of analysis. The top 5 identified (“known”) compounds included trimethylamine-N-oxide (TMAO) and trimethyllysine (TML), compounds linked to gut microbiota metabolism and whose levels have previously been associated with incident CVD risks (Koeth et al., 2013; Li et al., 2018; Tang et al., 2013; Wang et al., 2011), along with three diradylglycerophospholipids with tentative structural identification. Turning our attention to the candidate list of “unknowns”, the top MACE-associated candidate (high resolution m/z 265.1188) fulfilled all three screening inclusion criteria, and showed a hazard ratio (HR) 95% (Confidence interval (CI)) for incident (3 year) MACE risk of 2.69 (1.614.52); P<0.0001 (FIG. 1A,1B; Table S2). The m/z 265.1188 plasma analyte was subsequently unambiguously identified as phenylacetylglutamine (PAGln) by multiple approaches, including demonstrating identical high resolution MS/MS spectra and retention time with authentic synthetic standard material on multiple column stationary phases and chromatography conditions, with and without derivatization, both in plasma or fractionated plasma (FIG. 1C). The m/z 265.1188 plasma analyte was also shown to have identical mass spectra with the recently deposited MS/MS spectra of authentic synthetic PAGln deposited in MassBank of North America.

Untargeted metabolomics analyses are only semi-quantitative and require both verification and more quantitative analyses of candidates to corroborate observed associations. We therefore developed a stable isotope dilution LC/MS/MS assay to quantify PAGln in an independent (non-overlapping) Validation Cohort (n=4,000) of stable subjects undergoing elective diagnostic cardiac evaluation. Within the entire cohort, higher plasma PAGln levels were observed amongst subjects with T2DM (P=0.0002 vs. nondiabetics), as well as amongst subjects with MACE (P<0.0001 vs. nonMACE). Kaplan-Meier survival analyses revealed subjects with high PAGln levels have greater incident (3 years) MACE risks (FIG. 1D; P<0.0001 log rank for all groups examined). Specifically, subjects with PAGln levels in the fourth quartile (Q4) versus first quartile (Q1) demonstrate a significant increased risk of incident (3 year) MACE risks (all subjects (HR, 2.80; 95% CI, 2.17-3.61); T2DM (HR, 2.73; 95% CI, 1.84-4.05) and nondiabetics (HR, 2.18; 95% CI, 1.59-3.00); FIG. 1E). Moreover, higher PAGln levels remained an independent predictor of incident MACE risks following adjustments for traditional cardiac risk factors (FIG. 1E).

Gut Microbiota Participate in Production of PAGln and Phenylacetylglycine (PAGly) in vivo

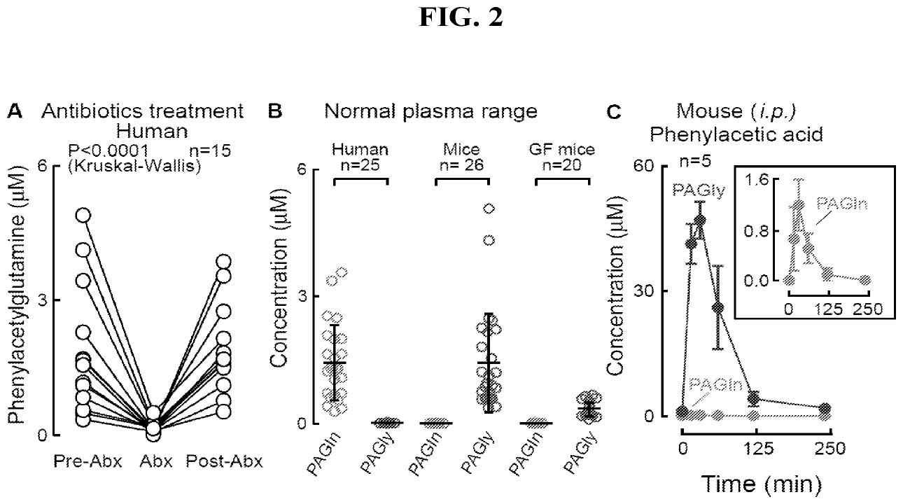

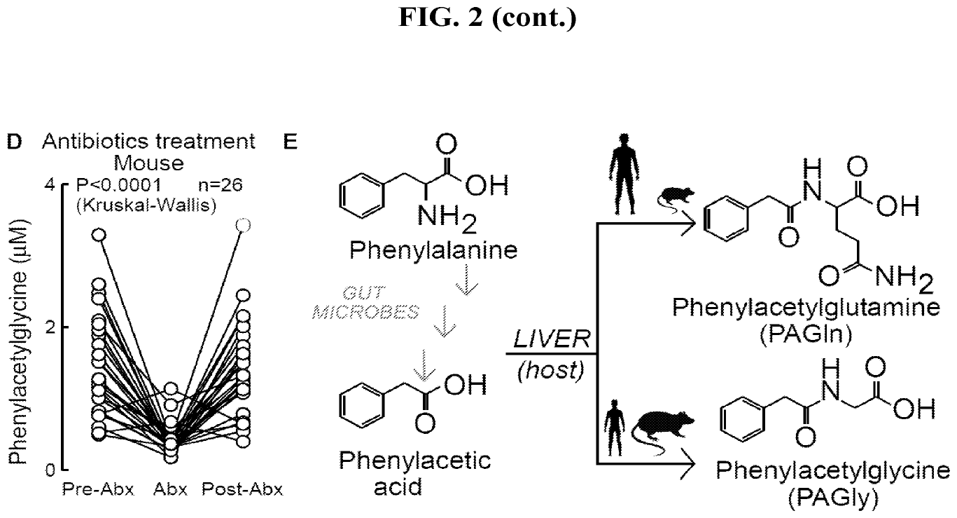

A gut microbial contribution to PAGln generation in humans was established by demonstrating marked suppression (P<0.0001) of systemic PAGln levels in subjects (n=15) in studies analyzing plasma recovered first at baseline (Pre-Abx), then following a 7 day course of oral poorly absorbed broad spectrum antibiotics cocktail (Abx) previously shown to suppress gut microbiota (Tang et al., 2013) and finally following a washout period (>3 weeks) to permit repopulation of conventional human gut commensals (Post-Abx) (FIG. 2A). Previous studies with either liver extracts or isolated enzymes have reported PAGln can be formed by phenylacetic acid conjugation to the amino acid glutamine by both human and rhesus monkey liver enzymes (Webster et al., 1976); while other studies have shown phenylacetic acid can be produced by bacterial fermentation of the essential amino acid phenylalanine in culture (Dodd et al., 2017). In addition to PAGln, phenylacetic acid conjugation to the amino acid glycine has also been noted, generating phenylacetylglycine (PAGly) (Gonzalez-Guardia et al., 2015; Wang et al., 2013). We therefore evaluated the physiological distribution of glycine versus glutamine derivatives of phenylacetic acid in apparently healthy subjects (n=25), as well as within mice, where mechanistic studies would be performed. Within humans, circulating levels of PAGln were found to be over an order of magnitude higher than PAGly; in contrast, PAGly levels in mice were an order of magnitude higher than PAGln (FIG. 2B). In addition, after intraperitoneal (i.p.) injection in mice, phenylacetic acid was shown to be predominantly metabolized into PAGly (FIG. 2C). Finally, comparisons of endogenous serum levels of PAGly in conventional versus germ free mice, or before versus after oral Abx treatment experiments, showed gut microbiota is a critical participant in PAGly formation from phenylacetic acid (FIG. 2B,D). Thus, in both humans and mice, PAGln and PAGly are produced in vivo via a metaorganismal pathway wherein dietary phenylalanine is converted into phenylacetic acid by gut microbiota, and then host conjugation reactions occur with either glutamine (preferred in humans) or glycine (preferred in rodents), producing PAGln and PAGly, respectively (FIG. 2E).

### PAGln Enhances Platelet Stimulus-Induced Calcium Release and Responsiveness to Multiple Agonists.

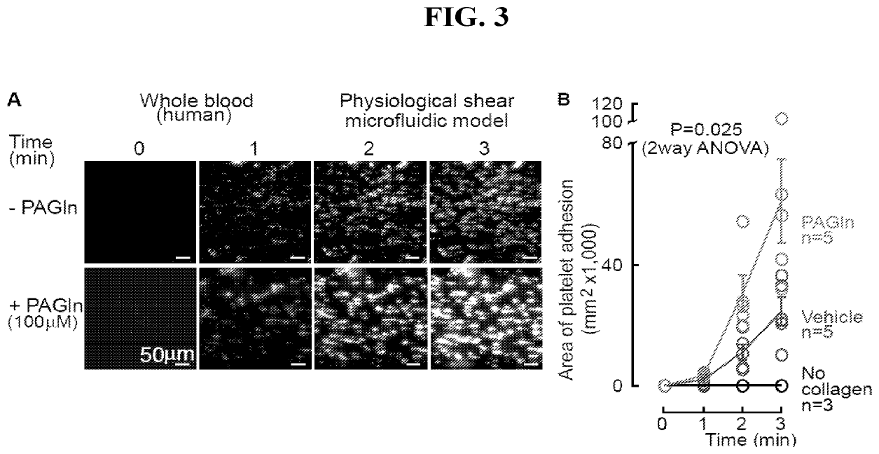

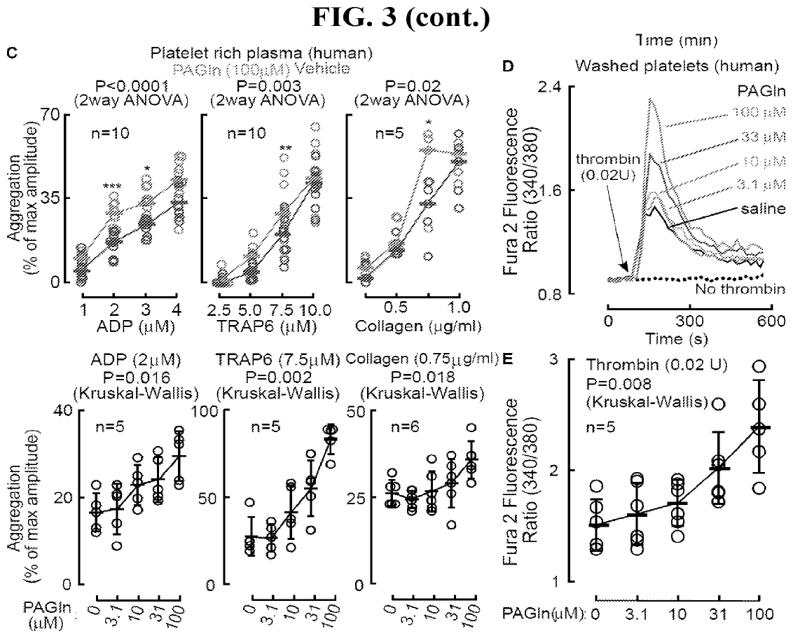

The observed positive association between PAGln levels and incident thrombotic events in humans (FIG. 1) suggested a potential effect of PAGln on platelet function and interactions with vascular matrix. We thus investigated whether PAGln impacted platelet adhesion to collagen surfaces in whole blood under physiological shear forces, as previously described (Zhu et al., 2016). Exposure (30 min) of whole blood recovered from healthy volunteers (all confirmed to have low (<4^{th} quartile PAGln levels) to physiologically relevant levels of PAGln substantially accelerated the rate of collagen-dependent platelet adhesion and spreading to surfaces observed under physiological shear flow (P=0.025; FIG. 3A,B). Further, brief (30 min) pretreatment of human platelet-rich plasma (PRP) with either PAGln (100 uM) versus saline (vehicle) revealed PAGln significantly enhanced stimulus-dependent platelet aggregation to sub-maximal concentrations of three different known platelet agonists: ADP, thrombin receptor-activated peptide (TRAP6) and collagen (FIG. 3C top; P<0.001 for ADP; P=0.003 for TRAP6 and P=0.02 for collagen). Moreover, when a fixed sub-maximal level of each of the platelet agonists (ADP, TRAP6, or collagen) was used, PAGln was observed to dose-dependently enhance the extent of platelet aggregation observed in PRP across the physiological range of concentrations of PAGln found in our clinical cohort (FIG. 3C bottom; P=0.016 for ADP; P=0.002 for TRAP6 and P=0.018 for collagen). These results collectively suggested PAGln might directly interact with platelets, enhancing stimulus-dependent rise in cytosolic Ca²⁺ concentrations. To directly test this, isolated human platelets were recovered from healthy donors, loaded with the calcium-selective dye Fura 2-AM, and real-time intracellular ionized cytosolic Ca²⁺ concentrations ([Ca²⁺]ᵢ) monitored before versus following thrombin-induced activation. Pre-incubation of platelets with PAGln had no effect on baseline [Ca²⁺]ᵢ levels (FIG. 3D). However, exposure to physiological levels of PAGln dose-dependently enhanced sub-maximal (0.02 U) thrombin-evoked augmentation of [Ca²⁺]ᵢ (FIG. 3D,E). Similar results were observed with PAGly, wherein addition to PRP augmented stimulus (ADP) dependent platelet aggregation response, and addition to washed human platelets provoked further rise in [Ca²⁺]ᵢ in response to sub-maximal thrombin exposure. Cumulatively, the above data demonstrate that the gut microbiota-dependent metabolites PAGln and PAGly significantly impact platelet function, enhancing platelet adhesion to collagen matrices, and enhancing platelet stimulus-dependent rise in [Ca²⁺]ᵢ and aggregation in response to agonists.

### PAGln Accelerates Platelet Dot Formation and Enhances Thrombosis Potential In Vivo

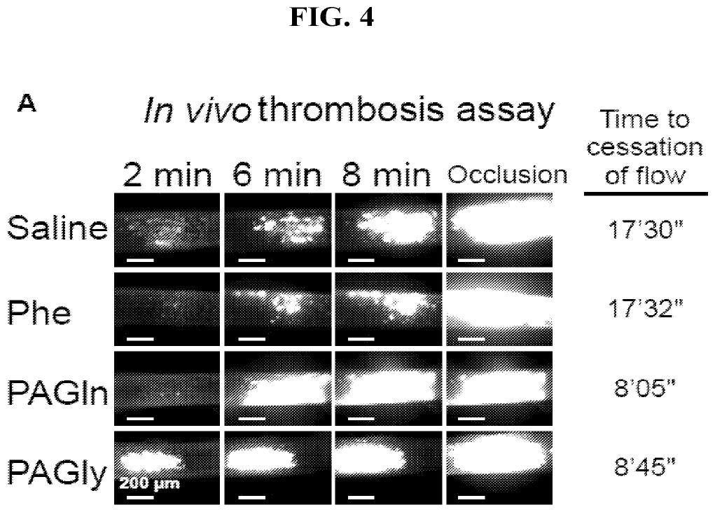

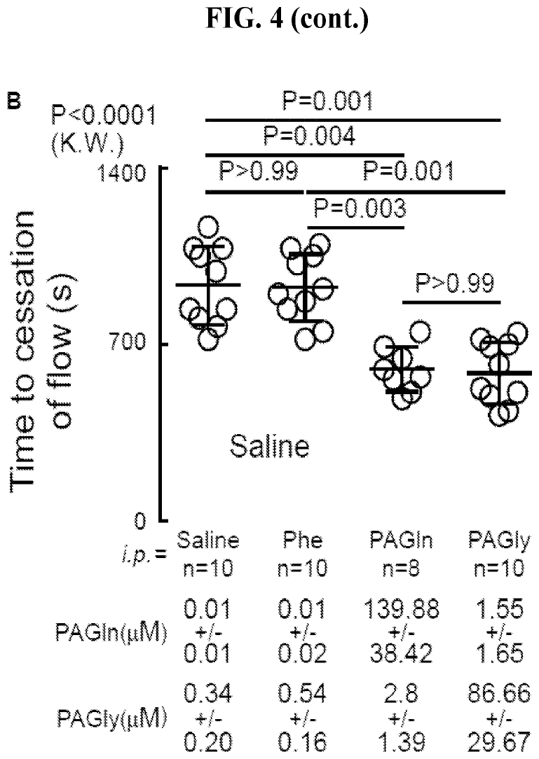

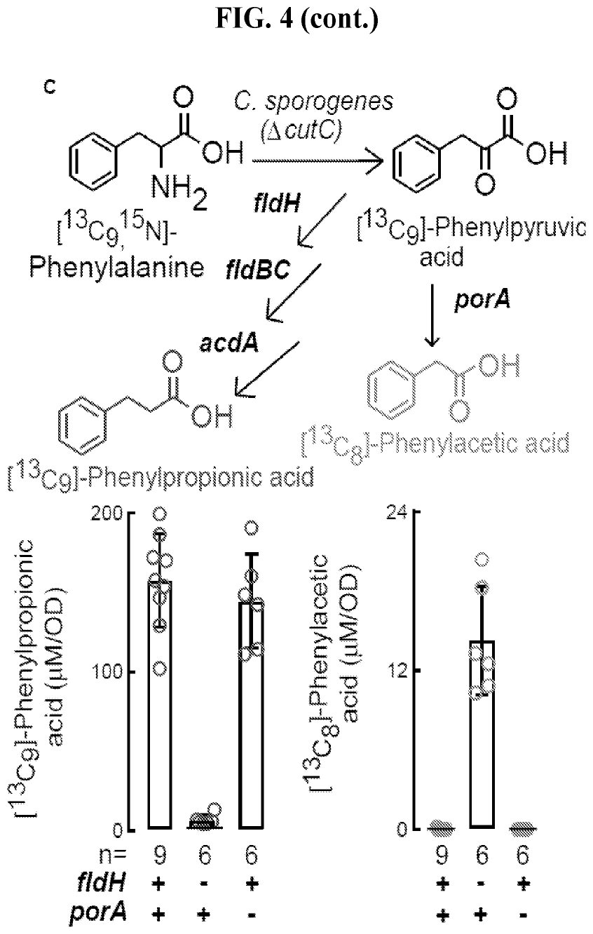

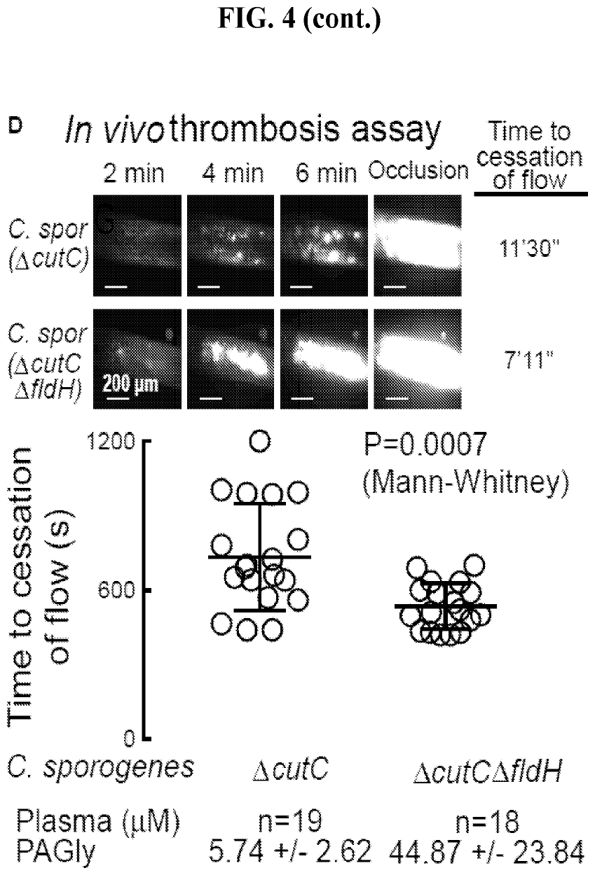

The impact of PAGln and PAGly on clot formation in vivo was examined using the FeCl₃-induced carotid artery injury model, a commonly used experimental approach to induce thrombosis. PAGln or PAGly were individually acutely raised to physiologically relevant levels (i.e. within 4^{th} quartile; FIG. 1) by i.p. injections in mice, and both the rate of platelet clotting, and the time to cessation of flow within the carotid artery quantified. Notably, both PAGln and PAGly each induced heightened platelet thrombus formation within the injured carotid artery (FIG. 4A), and correspondingly, reduced the time to cessation of flow following injury (i.e., the occlusion time), compared to mice alternatively treated with the nutrient precursor amino acid phenylalanine, or normal saline (vehicle) control (FIG. 4B).

Gut Microbial porA and fldH Modulate Host Thrombosis Potential in Vivo.

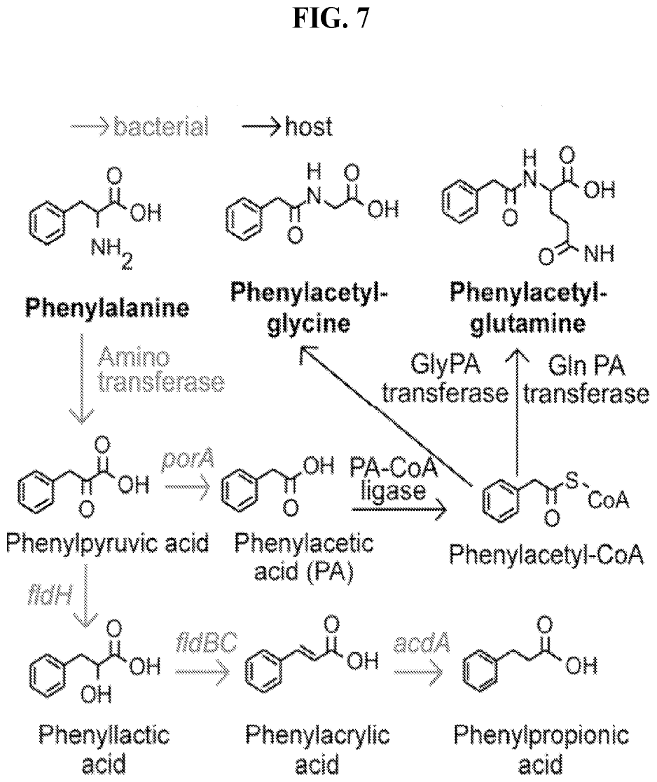

We next sought to test whether the gut microbial commensals and genes that contribute to PAGln/PAGly generation within hosts can modulate platelet function and thrombosis potential in vivo. Based on a combination of prior studies (Dodd et al., 2017; Elsden et al., 1976; Dickert et al., 2002), we illustrate in FIG. 7 a schematic of proposed metaorganismal microbial metabolic pathways for initial anaerobic conversion of dietary phenylalanine into either phenylacetic acid (oxidative pathway) or phenylpropionic acid (reductive pathway). These reactions are followed by host catalyzed amino acid (Gln or Gly) condensation reactions with phenylacetic acid forming PAGln and PAGly, and β-oxidation of phenylpropionic acid into benzoic acid and condensation with Gly forming hippuric acid. Recent studies by Dodd and colleagues (Dodd et al., 2017) reported a role for the porA gene in oxidative metabolism of phenylalanine to phenylacetic acid by the human commensal Clostridium sporogenes. In addition, Elsden and colleagues previously reported phenylpropionic acid was a major product of phenylalanine metabolism by clostridia (Elsden et al., 1976), and Dodd and colleagues also characterized a 15 kb gene cluster in C. sporogenes that harbors the phenyllactate dehydratase gene (fldABC) (Dickert et al., 2002), along with adjacent genes (e.g. fldH), that were shown to be critical to catalytic reductive metabolism of aromatic amino acids, including phenylalanine (Dodd et al., 2017). The scheme is also based on phenylacetic acid metabolism in humans and other mammals previously studied due to its use as an acute emergency treatment for hyperammonemia in patients with urea-cycle disorders, facilitating nitrogen removal as PAGln via urinary excretion (Brusilow et al., 1980; Webster et al., 1976). We therefore generated C. sporogenes mutants lacking either the porA or the fldH genes, as described under Methods. Because of the ability of C. sporogenes to generate trimethylamine, an alternative gut microbiota-derived metabolite that is a known precursor of the pro-thrombotic metabolite TMAO (Skye et al., 2018; Zhu et al., 2016), both porA or fldH deletion mutants were generated in a C. sporogenes ΔcutC background. To functionally characterize the mutants, they were cultured under anaerobic conditions in the presence of [¹³C₉, ¹⁵N₁]-phenylalanine and generation of [¹³C₈]-phenylacetic acid versus [¹³C₉]-phenylpropionic acid were determined by LC/MS/MS, as described in Methods. As shown in FIG. 4C, while C. sporogenes ΔcutC harboring both porA and fldH only generated isotope labeled phenylpropionic acid and not phenylacetic acid, the ΔcutCΔfldH mutant (still possesses functional porA) no longer generated phenylpropionic acid, but generated phenylacetic acid. In contrast, the ΔcutCΔporA C. sporogenes (still possesses functional fldH) mutant no longer generated phenylacetic acid, and only formed phenylpropionic acid. To directly test the impact of gut microbial phenylacetic acid generation on host in vivo thrombosis potential, we therefore colonized germ free mice with either the C. sporogenes ΔcutC or the C. sporogenes ΔcutCΔfldH mutants. Following microbial colonization, plasma levels of PAGly were determined by LC/MS/MS from serum samples recovered at time of in vivo thrombosis assessment using the FeCl₃ induced carotid artery injury model, as described under Methods. Colonization of C. sporogenes strains was confirmed by PCR of DNA extracted from cecal contents at the time of sacrifice post inoculation. As predicted by the in vitro culture data (FIG. 4C), colonization of the germ free mice with C. sporogenes ΔcutCΔfldH resulted in significantly higher levels of PAGly (P<0.001) than that observed in germ free mice colonized with C. sporogenes ΔcutC (FIG. 4D). Importantly, following arterial injury, both the rate of thrombus generation, and the time to cessation of flow within the injured vessel, were significantly reduced in the mice colonized with the C. sporogenes mutant producing higher levels of phenylacetic acid (i.e. ΔcutCΔfldH) and consequently, PAGly (FIG. 4D).

### PAGln Shows Saturable and Specific Binding to Cells, Suggesting Specific Receptor-Ligand Binding Interaction.

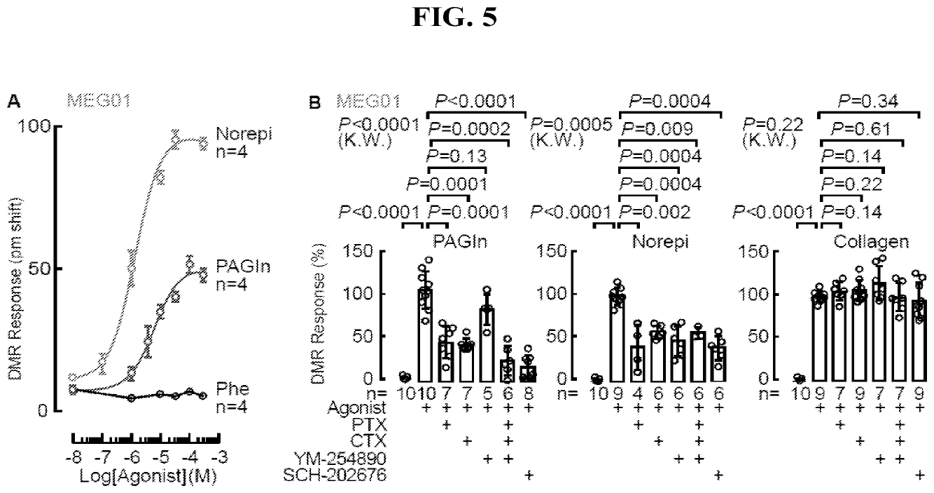

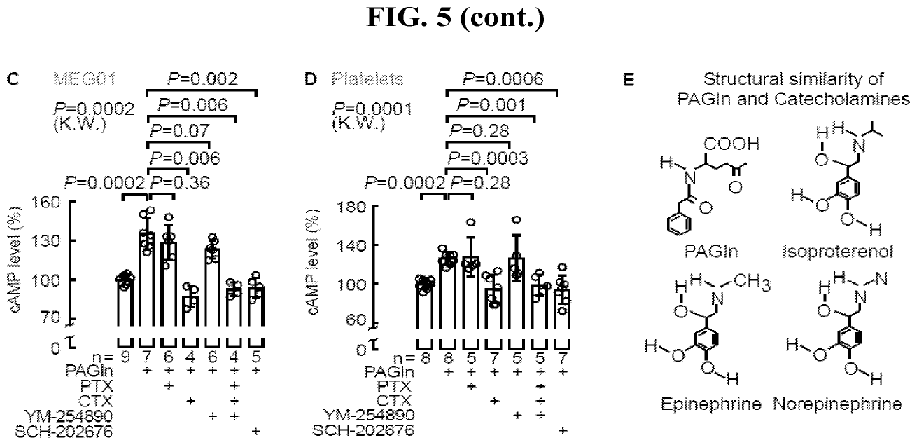

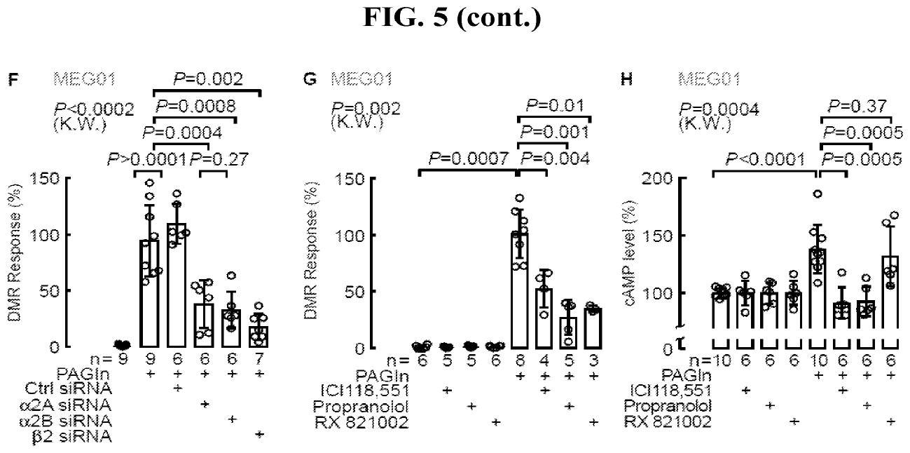

Since, PAGln directly fosters cellular effects at physiological levels, we next sought to decipher underlying molecular participants that might contribute to PAGln elicited cellular events. To test whether PAGln shows evidence of binding to a cellular receptor(s), we used dynamic mass redistribution (DMR), a label-free technology based on refractive index changes that enables real-time detection of ligand-dependent integrated cellular responses in live cells (Schroder et al., 2010; Schroder et al., 2011). Addition of PAGln to the human bone marrow-derived megakaryoblast cell line MEG01, a culturable precursor cell of platelets, resulted in a positive DMR response similar to known receptor binding ligands like norepinephrine and collagen, which bind to adrenergic receptors and the glycoprotein VI (GPVI) receptor, respectively, and acted as positive controls (Leitinger, 2011; Small et al., 2003). In contrast, the PAGln precursor (and structural analogue) phenylalanine, which does not bind to a legitimate cell receptor, displayed no DMR response (similar to vehicle control), and was used as negative control. Examination of increasing concentrations of potential binding ligands (PAGln vs norepinephrine vs phenylalanine) with MEG01 cells demonstrated saturable and specific DMR dose response curves, typical for a ligand-receptor interaction process, with PAGln and the positive control (norepinephrine) but not phenylalanine (FIG. 5A). In parallel studies, identical experimental studies examined PAGln binding with the human bone marrow derived erythroleukemia cell line HEL92.1.7, which also showed saturable and specific DMR dose-response signal with PAGln, like norepinephrine.

### PAGln Mediates Cellular Events Via G Protein-Coupled Receptors (GPCR)s

Since PAGln demonstrates receptor-ligand interaction properties with cells, we tested whether PAGln-induced DMR cellular responses were impacted by known GPCR signaling pathway modulators like pertussis toxin (PTX), cholera toxin (CTX) and YM-254890, which attenuate activation of Gα_{i/o}, Gαₛ and Gα_{q} subunit-mediated signaling pathways, respectively (Campbell and Smrcka, 2018; Milligan and Kostenis, 2006). Additionally, we examined PAGln-induced DMR cellular responses in the absence or presence of the agent SCH-202676, a global GPCR inhibitor that blocks both agonist and antagonist binding to a number of structurally diverse GPCRs that couple to Gα_{i/o}, Gαₛ and Gα_{q} proteins (Fawzi et al., 2001; Lewandowicz et al., 2006). As positive control, we used norepinephrine (Norepi) as a GPCR binding ligand, while collagen (a non-GPCR binding ligand) was used as negative control. The PAGln-induced DMR response in MEG01 cells was significantly reduced by pretreating the cells with either a combination of all three modulators (PTX, CTX, and YM-254890), or use of the global GPCR inhibitor SCH-202676, strongly implicating GPCR involvement in PAGln responses by the cells (FIG. 5B, left panel). Further, pretreatment of cells with either PTX or CTX, but not YM-254890, significantly suppressed the PAGln-induced DMR response, suggesting Gα_{i/o} and Gαₛ involvement (FIG. 5B, left panel). Moreover, examination of DMR responses to Norepi (positive control), which is known to signal through all three of the aforementioned G-protein subunits to variable degree (Hein, 2006), showed significant reduction in DMR signal upon incubations with each of the known GPCR modulator agents (FIG. 5B, middle panel). In contrast, the DMR signal with collagen (negative control), which binds to a non-GPCR receptor glycoprotein VI (GPVI) on platelets, remained unaffected by pretreatment of cells to each of the known GPCR modulator agents (FIG. 5B, right panel). GPCR involvement in PAGln-induced DMR responses were also indicated in HEL.92.1.7 cells, where a similar effect of these G-protein modulators was observed, though unlike in MEG01, the Gα_{q} modulator YM-254890 also displayed a significant reduction in PAGln induced DMR response.

The above data suggests PAGln-induced DMR responses in MEG01 and HEL92.1.7 cells appear to be mediated via saturable and specific responses to one or more GPCRs. We therefore examined whether PAGln elicited detectable changes in either intracellular cAMP or Ca²⁺, second messengers traditionally monitored in functional assays of GPCR pathways (Zhang and Xie, 2012). As we previously noted with platelets (FIG. 3D), in the absence of other agonists, PAGln alone had no observed direct effect on intracellular Ca²⁺ levels in either MEG01 or HEL92.1.7 cells (FIG. 5F). However, physiological levels of PAGln did elicit a modest (approx. 20-30%) but significant (P=0.0002 for each) transient (within 5 min) increase in intracellular level of cAMP in both MEG01 cells (FIG. 5C), and washed human platelets (FIG. 5D). Moreover, when either MEG01 or washed human platelets were each pretreated with known GPCR modulators, only CTX and the global GPCR inhibitor SCH-202676 inhibited PAGln-evoked cAMP production, suggesting PAGln exposure in these cells triggered Gas mediated activation of adenylylcyclase. Isoproterenol (ISO) was used as a positive control which predominantly binds to β2-adrenergic receptors that primarily couples to the Gas subunit in both MEG01 cells and in platelets (Koryakina et al., 2012). Collectively, these data indicate PAGln mediates cellular responses through GPCR(s) in multiple cells including isolated human platelets.

### The Gut Microbiota-Dependent Metabolite, PAGln, Acts Via Adrenergic Receptors (Adrenoceptors)

Given that PAGln mediates cellular responses via GPCR(s), we next sought to determine their identity. Nearly a 1000 distinct GPCRs exist within the human genome, so identifying which one(s) might respond PAGln is daunting. We noted, however, that PAGln itself has a core backbone structure of a phenylethylamine moiety (marked in highlight), similar to numerous catecholamines known to bind to the large multi-gene family of adrenergic receptors (ADRs) (FIG. 5E). We therefore hypothesized that PAGln may induce cellular signaling through adrenergic receptors focusing on ADR family members previously reported to be present on human platelets (α2A, α2B and β2 ADRs) (Anfossi and Trovati, 1996; Barnett et al., 1985; Colman, 1990). We initially performed loss of function studies in MEG01 cells using both genetic and pharmacological approaches. PAGln-induced DMR responses were monitored in MEG01 cells following transfection with control scrambled siRNAs or siRNA targeting either α2A, α2B or β2 ADRs. Importantly, we confirmed greater than 50% reduction in MEG01 cell mRNA levels of either ADR2A, ADR2B or ADRB2 following siRNA targeting of either α2A, α2B or β2 ADRs, respectively, but no reduction following control scrambled siRNAs. Notably, each of the siRNAs against the three ADRs demonstrated a parallel significant reduction in PAGln-induced DMR response in the MEG01 cells while control scrambled siRNA had no effect on DMR responses (FIG. 5F). Moreover, an additional positive control (DMR response of epinephrine, which binds to all the aforementioned platelet ADRs to various extent; Hein, 2006) showed significant reduction in DMR signal with siRNA knockdown of either α2A, α2B or β2 ADRs, but not with the control scrambled siRNA. Further, each of the same ADR targeting siRNAs showed no effect on MEG01 DMR responses following exposure to a non-ADR binding ligand, ATP, which served as an additional control.

In complementary studies, we used pharmacological inhibitors against the known ADRs in human platelets (α2A, α2B or β2 ADRs), namely, the selective β2-antagonist ICI118,551, the nonselective β-blocker propranolol, and the nonselective α2-antagonist RX821002 (Atwood et al., 2011; Bilski et al., 1983; O'Rourke et al., 1994). The DMR response induced by PAGln incubation with MEG01 cells was substantially reduced by each of the aforementioned pharmacologic inhibitors, while the inhibitors alone did not affect the DMR response (FIG. 5G). As positive control, the DMR response of the β2-agonist isoproterenol (ISO) was diminished in presence of ICI118,551, as well as with propranolol; and the DMR response of cell exposure to the α2-agonist B-HT933 was appropriately inhibited with RX821002. As additional control, the cellular DMR response to exposure to the non-ADR binding ligand ATP remained unchanged by the presence of any of the tested ADR inhibitors (ICI118,551, propranolol or RX821002). Finally, loss of function studies using pharmacologic inhibitors were also carried out to analyze cAMP production in both MEG01 cells and washed human platelets. ICI118,551 (β2 selective antagonist) and propranolol (nonselective β-blocker), but not RX821002 (nonselective α-antagonist) inhibited PAGln (5 min exposure) induced intracellular cAMP production in both MEG01 cells (FIG. 5H) and platelets. Similarly, intracellular cAMP production after ISO treatment in MEG01 and washed human platelets was reduced with ICI 118,551 and propranolol, but not with RX821002. Thus, both genetic and pharmacological loss of function studies confirm PAGln induces cellular responses via α2A, α2B and β2 ADRs.

### Gain of Function Studies Confirm the PAGln can Signal Via α2A, α2B and β2 ADRs

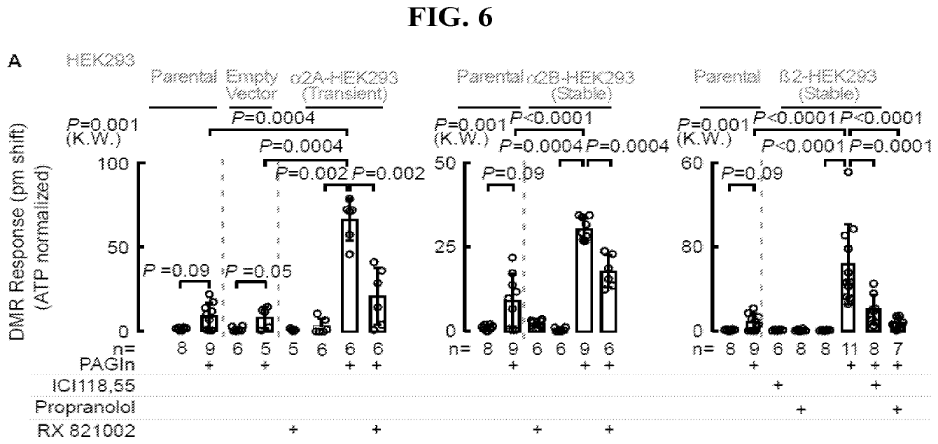

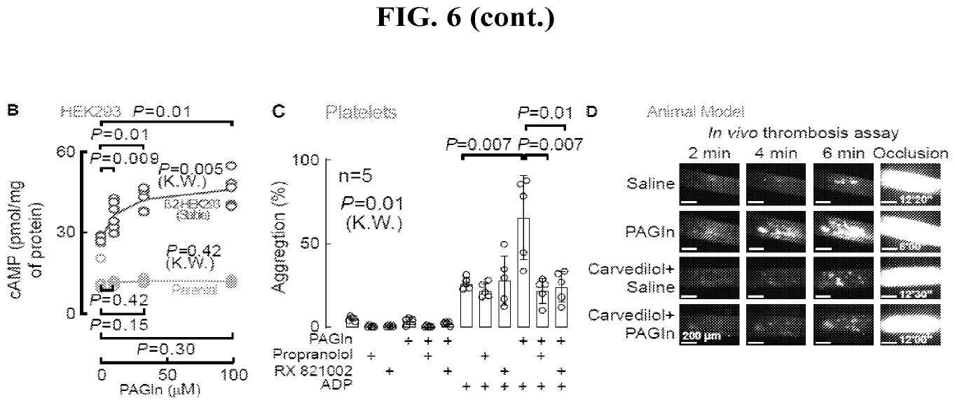

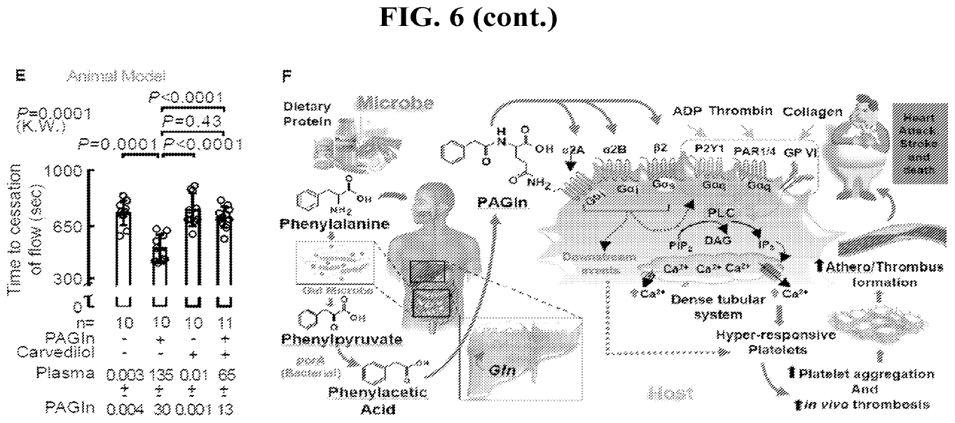

To further confirm the capacity of PAGln to function through ADRs, we carried out gain of function studies by individually over-expressing α2A, α2B and β2 ADRs in the human embryonic kidney cell line (HEK 293 cells), which were selected because of their low endogenous level of ADRs (Atwood et al., 2011). While incubation of either parental or empty vector-transfected HEK293 cells with PAGln elicited nominal DMR responses, enhanced PAGln-evoked DMR responses were observed with HEK293 cells either transiently transfected with the adra2a gene (α2A-HEK293), or stable overexpression of α2B ADR (α2B-HEK293) or β2 ADR (β2-HEK293) (FIG. 6A). Moreover, use of the above mentioned selective ADR pharmacological inhibitors reversed the PAGln-induced DMR responses in the corresponding ADR expressing cells (i.e. the PAGln-induced DMR responses in α2A-HEK293 and α2B-HEK293 cells were reversed with the nonselective α2-antagonist RX821002, and the PAGln-induced DMR responses in β2-HEK293 cells were attenuated by either the selective β2-antagonist ICI118,551, or the nonselective β-blocker propranolol (FIG. 6A)). As additional control studies, we performed similar experiments using the α2-agonist B-HT933 for α2A-HEK293 (transient) and α2B-HEK293 (stable) cells, and observed an elevated B-HT933-elicited DMR response as compared to parental HEK-293 cells while responses were attenuated by RX821002. Likewise, the β2-agonist ISO displayed enhanced DMR response in β2-HEK293 (stable) cell lines, that was inhibited with either ICI118,551 or propranolol. As an additional (negative) control, ATP-elicited DMR responses remained unaffected in all ADR transfected cells (α2A-HEK293 (transient), α2B-HEK293 (stable) and β2-HEK293 (stable) in the presence of the respective ADR inhibitors.

In complementary gain of function studies, we analyzed cAMP levels in parental HEK293 and β2-HEK293 (stable) cells in the absence versus presence of increasing concentrations of PAGln. Notably, parental HEK293 cells showed no PAGln-evoked change in intracellular cAMP levels (green line), whereas β2-HEK293 (stable) cells demonstrated dose-dependent significant increases in cytosolic cAMP levels (blue line) (FIG. 6B). Furthermore, intracellular levels of Ca²⁺ remained unaffected by PAGln exposure in all cells examined (parental HEK293, β2-HEK293 (stable) cells, empty vector (EV) transfected HEK293 cells, α2A-HEK293 (transient) cells and α2B-HEK293 (stable) cells). Thus, collectively, both loss of function and gain of function studies, employing either genetic or pharmacological approaches in multiple cell types, confirm that PAGln can induce cellular responses via α2A, α2B and β2 adrenergic receptors.

Platelet Hyperresponsiveness and Accelerated In Vivo Rate of Thrombus Generation Evoked by PAGln are Attenuated with Select ADR Inhibitors

The above studies suggest that some of the potential benefits of adrenergic receptor blocking agents in CVD may arise from antagonism of PAGln induced effects. Consistent with this, we observed exposure of platelet rich plasma (PRP) recovered from healthy volunteers with low PAGln levels to pathophysiologically relevant levels of PAGln augmented submaximal ADP-stimulated platelet aggregation responses. Concomitantly, PAGln exposure in the presence of either the nonselective beta-blocker propranolol, or the nonselective α2-antagonist RX821002, attenuated the PAGln induced hyperresponsiveness (FIG. 6c). Moreover, neither ADR inhibitor showed a direct effect on submaximal ADP-stimulated platelet aggregation response (but abolished the PAGln heightened platelet aggregation response; FIG. 6C). Thus, addition of each of the adrenergic receptor inhibitors reversed PAGln-elicited platelet hyperresponsiveness.

In another series of studies, we examined the effect of a widely used β-blocker (BBlkr) in clinical practice, carvedilol (Gupta et al., 2004), on PAGln induced enhancement in thrombus formation in vivo. Studies were performed as before (FIG. 4) examining the impact of PAGln (acutely elevated via i.p. injection) on FeCl₃ induced arterial injury provoked rate of platelet thrombus formation, and the time to vessel occlusion; however, this time using mice in the absence versus presence of carvedilol (1.5 g/kg for one week). Notably, in the absence BBlkr therapy, PAGln again both significantly accelerated the rate of platelet thrombus generation (FIG. 6D) and shortened the time to cessation of blood flow observed (FIG. 6E). However, the pro-thrombotic effect of PAGln on both clot formation rate and occlusion time were reversed by pretreatment of mice with carvedilol (FIG. 6D,E).

To both enrich our potential pool of candidate metabolic derangements associated with incident CVD event risks and enhance the likelihood of uncovering a novel pathway, we elected to initially focus our studies on a vulnerable patient population at known heightened CVD risks but via unclear mechanisms—diabetics, (i.e. the heightened CVD risks in T2DM are recognized to be, in part, via mechanisms distinct from traditional CVD risk factors and the degree of glucose control (Action to Control Cardiovascular Risk in Diabetes Study et al., 2008; Duckworth et al., 2009; Gerstein et al., 2001; Piarulli et al., 2009). We thus sought to identify circulating metabolites that were increased in T2DM versus non diabetic subjects, associated with incident development with CVD and its major adverse events (heart attack, stroke and death), and poorly correlated with indices of glycemic control (i.e. fasting glucose, HgbA1C, glucose/insulin ratio). Remarkably, many of the top candidates identified that met these criteria (both known and unknown structures at time of analysis), proved to be mechanistically linked to gut nucrobiota (e.g. PAGln. TMAO and TML; Koeth et al., 2013; Tang et al., 2013; Wang et al., 2011; Zhu et al., 2016). Moreover, PAGln (like TMAO and TML), was observed to be associated with incident CVD risks in both T2DM subjects and nondiabetic subjects alike. Thus, use of untargeted metabolomics coupled with functional studies has proven to be a valuable approach for discovery of both new pathways clinically and mechanistically linked to CVD, and interestingly, there appears to be a strong proportion of metabolic candidates/pathways linked to gut microbiome—the filter of our largest environmental exposure—what we eat (Aron-Wisnewsky and Clement, 2016; Brown and Hazen, 2018; Jonsson and Backhed, 2017; Koopen et al., 2016).

A scheme summarizing the metaorganismal origins of PAGln (and PAGly), its recognition by ADRs, and its overall relationship with CVD as revealed through the studies presented, is illustrated in FIG. 6f. Following ingestion of phenylalanine (Phe), the majority of the essential amino acid is absorbed in the small intestines, but unabsorbed Phe that reaches the large intestines can be metabolized by gut microbiota to form phenylpyruvic acid (the initial microbiota generated deamination product) and subsequently phenylacetic acid. Following absorption into the portal system, phenylacetic acid is readily metabolized in the liver to produce PAGln (major product in humans) and PAGly (major product in mice). PAGln has long been recognized as a synthetic product of phenylacetic acid and glutamine by liver and renal tissues of humans (Moldave and Meister, 1957), as well as a non-invasive probe of citric acid cycle intermediates in humans and primates (Yang et al., 1996). The present studies confirm a major role for gut microbiota in “endogenous” PAGln (and PAGly) generation in both humans and mice, as shown in studies with antibiotic cocktail to suppress gut microbiota, and corroborative studies using germ free versus conventionally reared mice. Notably, a limited number of recent studies have suggested an association between PAGln and some cardiometabolic phenotypes using semi-quantitative analyses. For example, PAGln levels were noted to be increased in subjects with end-stage renal disease and associated with mortality in one study (Shafi et al., 2015), while not so in another study (Shafi et al., 2017). Furthermore, elevated urinary levels of PAGln have recently been associated with obesity (Elliott et al., 2015), early renal function decline (Barrios et al., 2015: Poesen et al., 2016) and heightened levels of PAGln were also recently noted among diabetics (Loo et al., 2018: Urpi-Sarda et al., 2019). However, despite these reports, a mechanistic link between PAGln and CVD pathogenesis has not yet been reported.

Notably, the present studies suggest several mechanisms through which the clinical association between heightened PAGln and incident CVD risks may occur. First. PAGln was shown to impact thrombosis potential through multiple complementary approaches. Ex vivo cellular studies using whole blood, platelet rich plasma, and isolated platelets all indicate PAGln (and PAGly) enhance platelet function (enhanced stimulus dependent responsiveness to multiple agonist and intracellular calcium release). Furthermore, numerous in vivo studies reveal pathophysiologically relevant levels of PAGln or PAGly each facilitate both enhanced rate of thrombus formation and thrombosis potential in arterial injury models, including studies ranging from direct administration of either PAGln or PAGly, to microbial transplantation studies that modulate production of phenylacetic acid, the precursor of PAGln (and PAGly). Notably, microbial engineering and transplantation studies into gnotobiotic mice reveal that the relative activity of the gut microbial fldH gene cluster versus porA gene can regulate the overall amount of gut microbial production of phenylacetic acid (versus phenylpropionic acid), impacting PAGly production within the host (colonized GF mice). Critically, the balance between gut microbial activities of either the FldH vs PorA protein ultimately impacted the in vivo thrombosis potential in the colonized mice under gnotobiotic conditions, as monitored by both rate of thrombus formation and vessel occlusion time following arterial injury.

Another mechanism through which PAGln may impact CVD relevant phenotypes is via its newly demonstrated interactions with GPCRs, including ADRs. Direct biophysical studies indicated saturable and specific binding of PAGln to cells, indicative of a cell receptor—ligand interaction, and studies with multiple complementary known GPCR inhibitors modulate PAGln mediated cellular responses and down-stream signaling. A systematic series of genetic loss-of-function studies, and gain-of-function studies, as well as multiple corroborative pharmacological inhibitor and agonist studies, unambiguously show PAGln can signal via α2A, α2B and β2 ADRs, ADRs known to be expressed on platelets. Moreover, PAGln elicited pro-thrombotic effects in a murine model of arterial injury were observed to be reversed by treatment of mice with a widely used D-blocker in clinical practice. In addition, studies with either specific ADR siRNA knockdown or ADR antagonists were both shown to block PAGln induced pro-thrombotic phenotypes, further indicating PAGln can promote cellular signals via ADRs. β Blocker therapy is known to foster numerous clinical benefits in some subjects with CVD (e.g. reduced risks of heart attack, stroke, heart failure and death: Black et al., 2010; Bumett et al., 2017; Witte et al., 2018).

ADRs are widely expressed on virtually every cell type, and known to play not only important roles in vascular and myocardial function, but more broadly, the regulation of body homeostasis in both health and pathologic states. They effect myocardial contraction, blood pressure, lung airflow, and stimulate the sympathetic nervous system and central nervous system functions (Amrani and Bradding, 2017: Hein and Kobilka, 1997: Lefkowitz et al., 2000; Rockman et al., 2002). Activation of the adrenergic system also has profound effect on metabolism and prolonged activation can lead to insulin resistance, alterations in glucose and fatty acid metabolism and mitochondrial dysfunction (Ciccarelli et al., 2013: Fu et al., 2017).

### Example 2

### Detection of PAG, TMAO, and TML

This Example describes the combined detection of PAG, TMAO, and TML for detecting risk of major adverse cardiac disease (MACE) at 3 and 5 years in a large cohort of 2138 subject. The baseline characteristics of the subjects is presented in Table 1 below.

TABLE 1
Baseline Characteristics According to Number of Elevated Markers (n = 2,138)

| | All | | 0 | | 1 | | 2 | | 3 | | |
|---|---|---|---|---|---|---|---|---|---|---|---|
| Characteristics | (n = 2138) | | (n = 742) | | (n = 640) | | (n = 415) | | (n = 342) | | p |
| Age (years) | 63 ± 11 | | 60 ± 11 | | 62 ± 11 | | 65 ± 11 | | 68 ± 10 | | <0.001 |
| Male, n (%) | 65.2 | | 64.4 | | 67 | | 65.1 | | 63.6 | | 0.679 |
| Diabetes mellitus (%) | 40.8 | | 32.7 | | 37.2 | | 45.1 | | 60.1 | | <0.001 |
| Hypertension (%) | 72.7 | | 69.2 | | 70 | | 73.7 | | 84.3 | | <0.001 |
| Former/Current | 64.8 | | 61.5 | | 67.7 | | 66.7 | | 64.2 | | 0.083 |
| smokers (%) | | | | | | | | | | | |
| Prior coronary | 74.5 | | 70.7 | | 73.8 | | 76.3 | | 82.1 | | 0.001 |
| artery disease (%) | | | | | | | | | | | |
| CVD (%) | 80.6 | | 77.8 | | 80.2 | | 81.4 | | 86.8 | | 0.006 |
| Framingham ATP III | 9 | (6-12) | 8 | (6-11) | 9 | (6-12) | 10 | (7-12) | 10 | (8-13) | <0.001 |
| Risk Score | | | | | | | | | | | |
| LDL cholesterol | 96 | (79-116) | 99 | (82-118) | 97 | (81.8-119) | 94 | (74-112) | 92 | (73-110) | <0.001 |
| (mg/dL) | | | | | | | | | | | |
| HDL cholesterol | 33.9 | (28.2-40.6) | 34.8 | (29.7-41.2) | 33.3 | (28-39.7) | 34 | (27.8-41) | 32.2 | (26.9-40.1) | 0.001 |
| (mg/dL) | | | | | | | | | | | |
| Triglycerides | 123 | (87-178) | 120.5 | (87.2-169) | 123 | (85-178) | 122 | (88-181.5) | 128 | (95-183) | 0.269 |
| (mg/dL) | | | | | | | | | | | |
| hsCRP | 2.6 | (1.1-6.3) | 2.3 | (1-5.7) | 2.3 | (1-5.6) | 2.6 | (1.1-6) | 3.8 | (1.4-8.8) | <0.001 |
| eGFR | 86.7 | (72.3-96.4) | 94 | (86-101.3) | 88.4 | (78.1-96.2) | 80.4 | (65.5-90.7) | 58.8 | (44.8-75.7) | <0.001 |
| ApoB | 82 | (70-97) | 84 | (71-98) | 83 | (70-98) | 81 | (69-94) | 80.5 | (67-92.2) | 0.007 |
| ApoA1 | 117 | (104-133) | 120 | (106-135) | 116 | (104-131) | 117 | (104-135) | 114 | (100-130) | 0.007 |
| ACE inhibitors or | 50.1 | | 44.3 | | 48 | | 54.5 | | 61.6 | | <0.001 |
| ARBs (%) | | | | | | | | | | | |
| Beta blocker (%) | 63 1 | | 62 | | 61.6 | | 60.7 | | 71.6 | | 0.006 |
| Statins (%) | 60.7 | | 62 | | 61.6 | | 58.3 | | 59.2 | | 0.569 |
| Aspirin (%) | 75.1 | | 75.7 | | 75.3 | | 76.4 | | 71.8 | | 0.48 |
| hsTnT | 13.6 | (8.2-26.1) | 11.7 | (7-19.6) | 12.4 | (7.5-22.1) | 15.2 | (8.9-28) | 19.6 | (12.2-38.1) | <0.001 |
| proBNP | 240.7 | (88.7-713.1) | 161.8 | (68.7-452.4) | 211 | (74.4-583.2) | 284.7 | (123.2-788.3) | 725.8 | (228.7-1929) | <0.001 |
| Trimethylamine | 4 | (2.5-6.6) | 2.5 | (1.8-3.2) | 3.7 | (2.7-5.2) | 6.2 | (4.7-9) | 9.3 | (6.9-13.6) | <0.001 |
| N-oxide (μM) | | | | | | | | | | | |
| Trimethyllysine | 0.5 | (0.4-0.7) | 0.4 | (0.4-0.5) | 0.6 | (0.4-0.7) | 0.6 | (0.5-0.8) | 0.8 | (0.7-1.1) | <0.001 |
| (μM) | | | | | | | | | | | |
| Phenoacetylglutamine | 3.1 | (1.8-5) | 1.9 | (1.3-2.7) | 2.9 | (1.9-4.2) | 4.5 | (3.3-6.1) | 7 | (5.3-10.6) | <0.001 |
| (μM) | | | | | | | | | | | |

Table 2 below presents the Unadjusted and Adjusted Hazard Ratios for Major Adverse Cardiac Events at 3 Years and All-Cause Mortality at 50 Years Stratified by Number of Elevated Markers (n=2,138).

TABLE 2

| | | | | |
|---|---|---|---|---|
| Markers Elevated | 0 | 1 | 2 | 3 |

Death, Myocardial Infarction & Stroke at 3 Years

| | | | | | | | |
|---|---|---|---|---|---|---|---|
| Event (%) | 67/742 = 9.03% | 86/640 | (13.44%) | 70/415 | (16.87%) | 103/341 | (30.21%) |
| Unadjusted | 1 | 1.51 | (1.10-2.09)* | 1.93 | (1.38-2.70)** | 3.75 | (2.76-5.10)** |
| Adjusted | 1 | 1.29 | (0.94-1.79) | 1.36 | (0.96-1.92) | 1.89 | (1.30-2.75) |

All-cause Mortality at 5 Years

| Event (%) | 52/742 = 7.01 | 76/640 = 11.88 | | 69/415 = 16.63 | | 107/341 = 31.38 | |
|---|---|---|---|---|---|---|---|
| Unadjusted | 1 | 1.75 | (1.23-2.48)** | 2.49 | (1.74-3.57)*** | 5.20 | (3.74-7.25)*** |
| Adjusted (2) | 1 | 1.39 | (0.97-1.99) | 1.52 | (1.04-2.22)* | 2.06 | (1.38-3.07)*** |

Adjusted Multivariate model adjusting for age, sex, systolic blood pressure, LDL-cholesterol, HDL-cholesterol, diabetes, smoking, and log-transformed estimated glomerular filtration rate. *p < 0.05; **p < 0.01, ***p < 0.0001

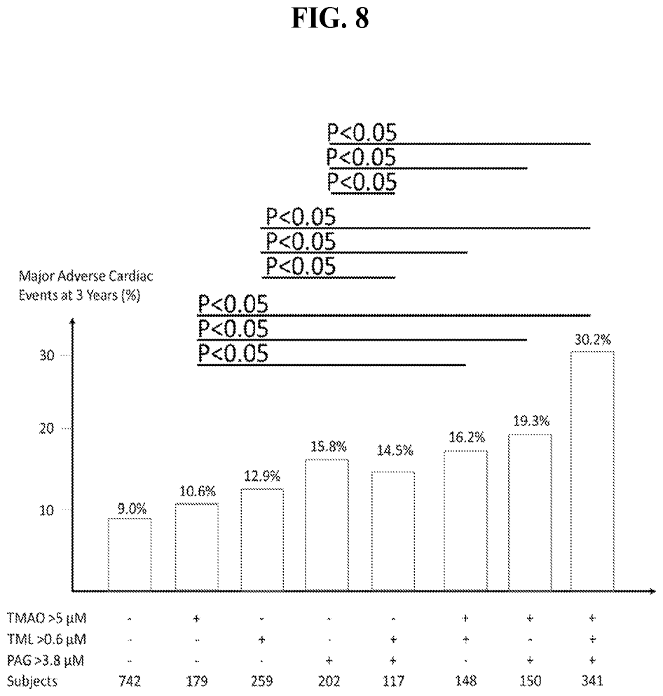

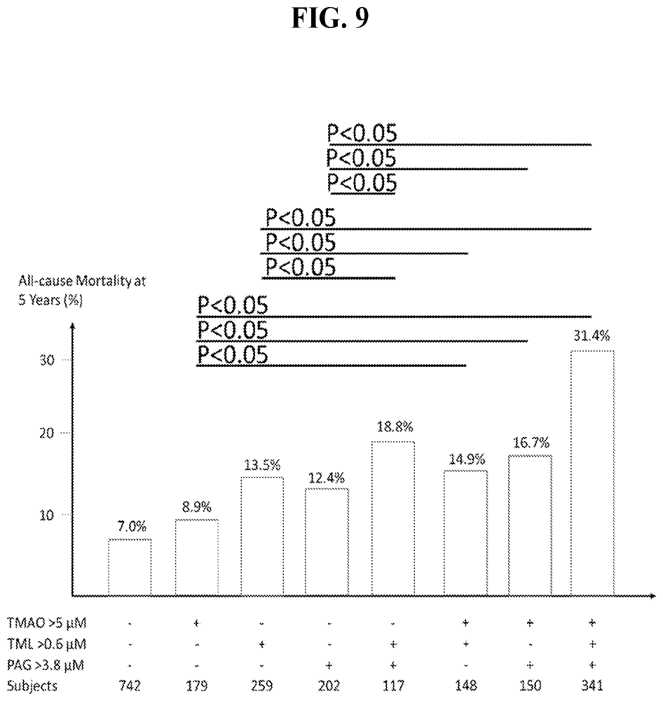

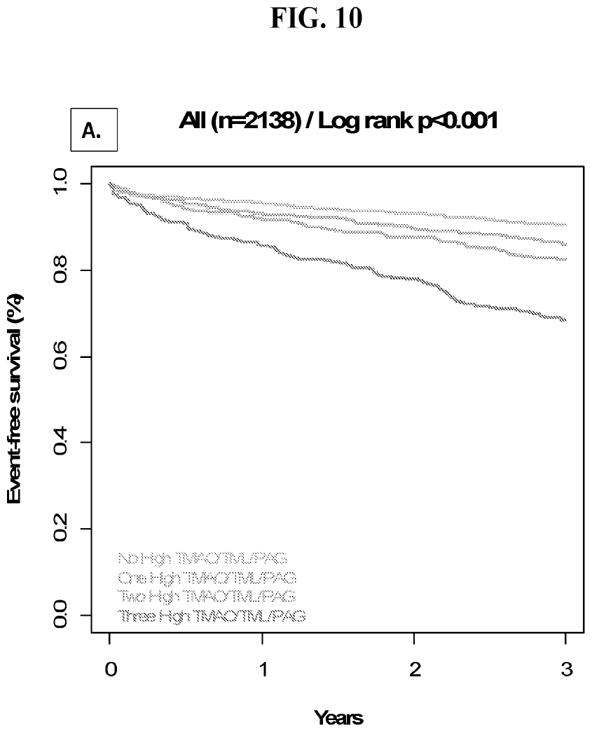

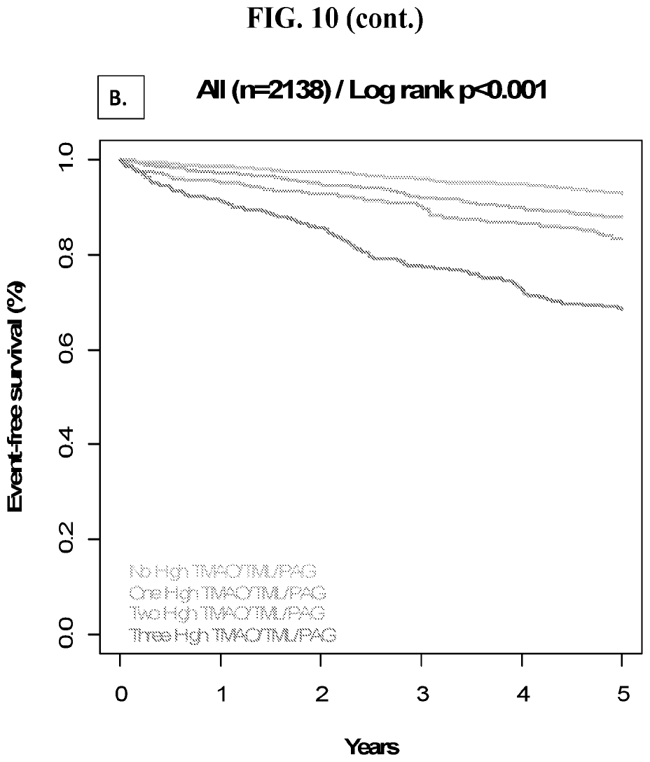

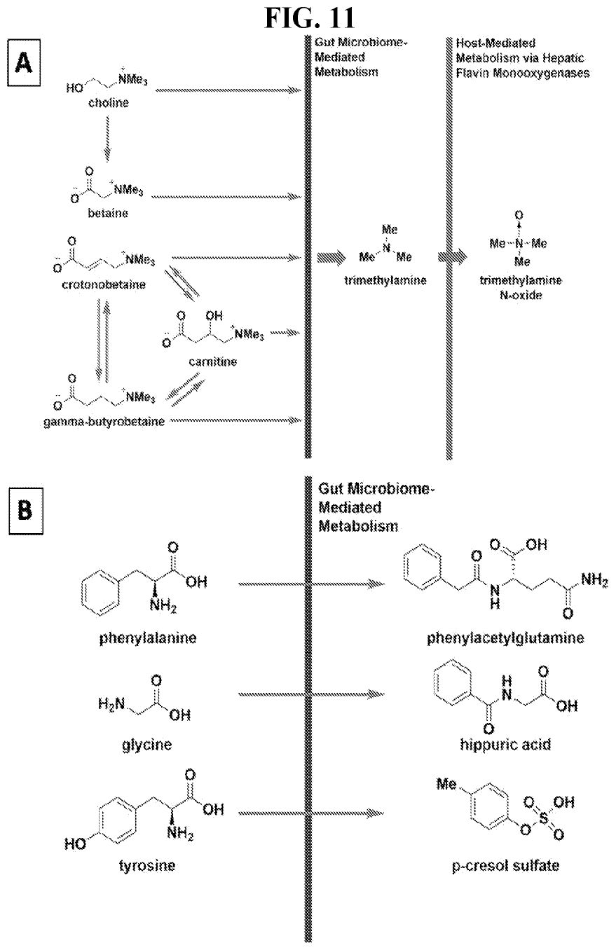

FIG. 8 shows a graph of MACE at 3 years based on single markers and combined PAGln, TMAO, and TML. FIG. 9 shows the same graphs except for all causes of mortality at 5 years. FIG. 10 shows even free survival at 3 years (FIG. 10a) and 5 years (FIG. 10b) for single and multiple markers.

### Example 3

### Gut Microbiome Metabolites and Risk of Lethal Prostate Cancer: Prospective Analysis of a PLCO Cohort

The gut-microbiota-dependent metabolism of trimethylamine-associated nutrients and amino acids has been associated with cardiovascular disease and cancer. However, the association of these compounds and metabolites with lethal prostate cancer remains unknown. The objective of this Example is to evaluate the association of baseline serum levels of trimethylamine precursor nutrients, trimethylamine N-oxide, and gut-microbiota-derived amino acid derivatives associated with lethal prostate cancer risk. A nested case-control design was employed to determine if differences in baseline choline, carnitine, betaine, γ-butyrobetaine, crotonobetaine, trimethylamine N-oxide, phenylacetylglutamine (PAG), hippuric acid, and p-cresol sulfate levels are associated with incident lethal prostate cancer. Cases were randomly matched to controls at a 1:3 ratio on the basis of age, race, time of baseline blood draw, and enrollment date. Baseline serum samples were collected between November 1993 and July 2001 as part of the Prostate, Lung, Colorectal, and Ovarian (PLCO) Cancer Screening trial. Screening was completed in 2006, and mortality data were collected through 2015. 173 cases and 519 controls without lethal PCa were analyzed. Samples were collected from men, aged 55-74, assigned to the intervention arm of the PLCO trial not previously diagnosed with prostate cancer. Relative to those in the first quartile, cases with higher baseline levels of choline (Q3 OR: 2.37, 95% CI: 1.35-4.17; Q4 OR: 2.19, 95% CI: 1.23-3.90; P-trend: 0.005) and betaine (Q3 OR: 2.13, 95% CI: 1.22-3.70; Q4 OR: 1.86, 95% CI: 1.05-3.30; P-trend: 0.11) exhibited increased odds of developing lethal prostate cancer after adjusting for BMI and PSA. This association was not consistently demonstrated for other trimethylamine-associated compounds including carnitine, γ-butyrobetaine, crotonobetaine, and trimethylamine N-oxide. Higher baseline serum levels of phenylacetylglutamine (Q3: 2.54, 95% CI: 1.41-4.58; Q4: 2.55, 95% CI: 1.40-4.64; P-trend: 0.003), a gut-microbiome metabolite of phenylalanine, were also associated with lethal prostate cancer, though elevations in hippuric acid and p-cresol sulfate were not.

### Methods:

### Prostate, Lung, Colorectal, and Ovarian (PLCO) Cohort

The PLCO cancer screening trial is a large, population-based, randomized controlled trial designed to evaluate effects of cancer screening in men and women between the ages of 55 and 74.²⁵ Between November 1993 and July 2001, ten screening centers in the United States enrolled 76,685 men, who were randomized to an intervention arm or a control arm. Men in the intervention arm received screening for PCa via PSA and digital rectal exam for the first six years of the trial and were followed for at least seven years thereafter. Trial data were collected through December 2009 and included baseline blood sample collection (prior to cancer diagnosis), demographic information, and screening results. The primary endpoint of this trial is cancer-specific mortality, though multiple secondary endpoints related to cancer screening and morbidity were examined. Through 2015, data were collected on participant deaths through the administration of the Annual Study Update questionnaire, by physician or family report, or by performing National Death Index Plus searches. A Death Review Process was then performed to evaluate all deaths and determine if a PLCO cancer was responsible.²⁶

### Nested Case-Control Study Design

We employed a nested case-control design. Baseline serum specimens from 173 lethal PCa cases and 519 controls without lethal PCa were analyzed. Samples were collected from men assigned to the intervention arm of the PLCO trial not previously diagnosed with PCa. The 173 cases represent all PCa deaths in the intervention arm for which prediagnostic samples were available. At the time of death, cases were matched at a 1:3 ratio with randomly selected living subjects in the corresponding risk set on the basis of race, age (within 2 years), time of baseline blood draw (within 6 months), and enrollment date (within 1 year). This study was designed to identify potential drivers of lethal disease, hence the focus on lethal PCa using strict risk set sampling.

Serum levels of choline, carnitine, betaine, γ-butyrobetaine, crotonobetaine, TMAO, PAGln, hippuric acid, and p-cresol sulfate levels were quantified from a single baseline measurement of analyte levels in serum from apparently healthy subjects who were then prospectively followed for incident development of PCa. Study approval was obtained by the National Cancer Institute and the Cleveland Clinic Institutional Review Board.

### Reagents for Metabolomic Analysis

d₉-[N,N,N-trimethyl]choline (d₉-choline), L-carnitine:HCL (methyl-D3, d₃-carnitine), d₉-trimethylamine N-oxide (d₉-TMAO), hippuric acid (benzoyl-D5, d₅-hippuric acid), and p-cresol sulfate potassium salt (D7, d₇-p-cresol sulfate) were purchased from Cambridge Isotope Laboratories (Andover, Mass.). p-Cresol sulfate (potassium salt) was purchased from Cayman Chemical (Ann Arbor, Mich.). d₉-[N,N,N-trimethyl]-betaine (d₉-betaine) and Nα-(Phenyl-d5-acetyl)-L-glutamine (d₅-phenylacetylglutamine) were purchased from C/D/N Isotopes (Quebec, Canada). d₉-[N,N,N-trimethyl-γ-butyrobetaine (d₉-γ-butyrobetaine) and d₉-[N,N,N-trimethyl]-crotonobetaine (d₉-crotonobetaine) were synthesized as previously described.²⁷⁻²⁹ All other reagents were purchased as HPLC grade from either Sigma-Aldrich (St. Louis, Mo.) or Fisher Scientific Chemicals (Pittsburgh, Pa.).

### Metabolomics Analysis

20 μl of plasma was mixed with 80 μl of cold methanol containing an isotope-labeled internal standard mix composed of d₉-choline, d₉-TMAO, d₉-betaine, d₃-catnitine, d₉-γ-butyrobetaine, d₉-crotonobetaine, d₇-p-cresol sulfate, d₅-phenylacetylglutamine and d₅-hippuric acid (each 5 μM). The mixture was vortexed and centrifuged at 20,000 g at 4° C. for 10 minutes. 0.2 μl of supernatant was injected onto Silica (150×2 mm, 00F-4274-B0, Phenomenx) at a flow rate of 0.25 ml/min using a Vanquish high-performance liquid chromatography (HPLC) system interfaced with a Thermo Quantiva mass spectrometer (Thermo Scientific) and a TSQ Quantiva Triple Quadrupole mass spectrometer (Thermo Scientific). A liquid chromatography (LC) gradient generated from two solvents (A: 0.1% propionic acid in water, B: 0.1% acetic acid in methanol) at a flow rate of 0.25 ml/min was used to resolve analytes. Samples were run sequentially and continuously on the LC/MS/MS system, with laboratory personnel blinded to the case status of the samples.

The targeted metabolites and isotope labeled internal standards were monitored using electrospray ionization (ESI) in positive-ion mode (except for p-cresol sulfate using negative-ion mode) with multiple reaction monitoring (MRM) of precursor and characteristic product ion transitions as previously reported.^{30,31} Parameters for the ion monitoring were optimized for individual metabolites and internal standards. Argon was used as the collision-induced dissociation (CID) gas and nitrogen (99.95% purity) was used otherwise.

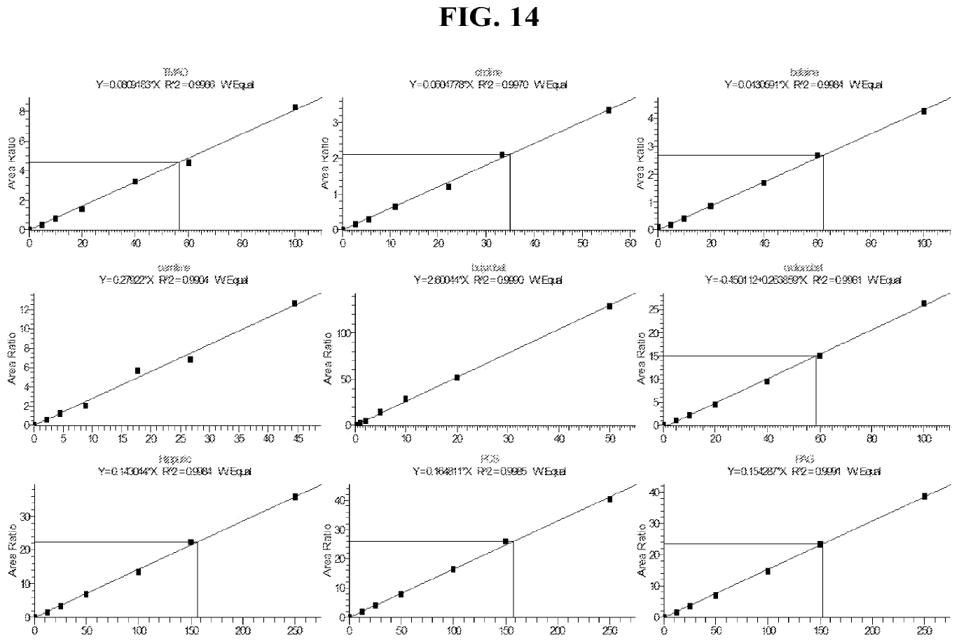

Various concentrations of standards were mixed with fixed concentrations of an isotope-labeled internal standard mix to prepare the calibration curves for quantification of the targeted analytes. The standard curves for quantitation of choline, carnitine, betaine, betaine, γ-butyrobetaine, crotonobetaine. PAGln, hippuric acid, and p-cresol sulfate are shown in FIG. 14. Error for the quantification curves was less than 5% for all analytes, demonstrating the reliability of this analytic approach.

### Statistical Analysis

Serum measurements were analyzed by quartile, with the distribution of analyte levels among the controls used to determine quartile (Q) thresholds. Multivariable conditional logistic regression analysis was used to assess the association of analyte levels with lethal PCa after conditioning on case status and adjusting for PSA and BMI. The odds ratio (OR) and 95% confidence interval (95% CI) of developing lethal PCa was reported for each quartile of nutrient and metabolite levels, with the first quartile (Q1) serving as the reference. Trend of increasing ORs was also assessed based on quartile medians using the Cochran-Armitage test. For other comparisons between cases and controls, median and interquartile ranges (IQR) or counts and percentages were reported, though significance was based on univariate conditional logistic regression where p-values are provided. All analyses were conducted using R (version 3.6.3) using a significance threshold of 0.05.

### Results

173 lethal PCa cases and 519 controls, well matched by age and race, were analyzed (Table 3).

TABLE 3
Baseline Clinical and Pathological Characteristics^{a}

| | Controls | | Cases | | |
|---|---|---|---|---|---|
| | n = 519 | | n = 173 | | P-value^{b} |
| Age, years, median (IQR) | 66 | (61-70) | 66 | (61-70) | matching factor^{c} |
| Race | | | | | matching factor^{c} |
| White | 477 | (91.9) | 159 | (91.9) | |
| Black | 33 | (6.4) | 11 | (6.4) | |
| Other | 9 | (1.7) | 3 | (1.7) | |
| BMI, kg/m\hat{ }2, median (IQR) | 26.6 | (24.7-29.0) | 26.9 | (25.2-29.6) | 0.03 |
| PSA nearest diagnosis, ng/mL | | | | | |
| Median (IQR) | 5.8 | (4.5-8.2) | 7.3 | (4.9-16.3) | 0.06 |
| <10 | 71 | (83.5) | 113 | (65.3) | 0.04 |
| 10-20 | 11 | (12.9) | 22 | (12.7) | 0.94 |
| >20 | 3 | (3.5) | 38 | (22.0) | 0.01 |
| Clinical T stage | | | | | |
| T1 | 53 | (10.2) | 55 | (31.8) | 0.007 |
| T2 | 30 | (5.8) | 94 | (54.3) | 0.16 |
| T3 | 0 | (0) | 10 | (5.8) | >0.99 |
| T4 | 0 | (0) | 6 | (3.5) | >0.99 |
| Unknown or not evaluated^{d} | 436 | (84.0) | 8 | (4.6) | 0.70 |
| Clinical N stage | | | | | |
| N0 | 63 | (12.1) | 112 | (64.7) | 0.49 |
| N1 | 0 | (0) | 6 | (3.5) | >0.99 |
| Nx | 20 | (3.9) | 55 | (31.8) | 0.67 |
| Unknown or not evaluated^{d} | 436 | (84.0) | 0 | (0) | >0.99 |
| Clinical M stage | | | | | |
| M0 | 69 | (13.3) | 113 | (65.3) | 0.03 |
| M1 | 1 | (0.2) | 38 | (22.0) | 0.003 |
| Mx | 13 | (2.5) | 0 | (0) | 0.43 |
| Unknown or not evaluated^{d} | 436 | (84.0) | 22 | (12.7) | >0.99 |
| Gleason Score on Biopsy | | | | | |
| 6 | 39 | (7.5) | 34 | (19.7) | 0.002 |
| 7 | 16 | (3.1) | 43 | (24.9) | 0.95 |
| 8 | 10 | (1.9) | 31 | (17.9) | 0.23 |
| 9 | 5 | (1.0) | 36 | (20.8) | 0.02 |
| 10 | 0 | (0) | 7 | (4.0) | >0.99 |
| Unknown or not evaluated^{d} | 449 | (86.5) | 22 | (12.7) | 0.48 |

Abbreviations: BMI, body mass index; PSA, prostate-specific antigen; IQR, interquartile range
^{a}Data are presented as number (percentage) of cases or controls unless otherwise specified
^{b}Significance testing performed using univariate conditional logistic regression
^{c}Patient factors used to match subjects at the time of case mortality as part of strict risk set sampling
^{d}Number reported represents cases or controls who underwent prostate cancer staging evaluation with missing clinical information, or controls who appropriately avoided biopsy and/or staging evaluation

A minority of controls were diagnosed with non-lethal PCa (16.4% 185/5191) by the end of the observation period. Median PSA levels nearest PCa diagnosis were similar between cases and controls (7.3 vs. 5.8 ng/dL, P=0.06). A majority of lethal prostate cancers were diagnosed at clinical stage T2, compared to a minority of the nonlethal cancers (57% 194/1651 vs. 36% 130/831).

Using conditional univariate logistic regression modeling, median baseline levels of TMA-associated nutrients were compared (Table 4).

TABLE 4
Distribution of Serum Analyte Levels and Assessment of Trend^{a}

| Metabolite, | Controls | | Cases | | | |
|---|---|---|---|---|---|---|
| uM | (n = 519) | | (n = 173) | | P-value^{b} | P-trend^{c} |
| Choline | 13.1 | (11.0-16.0) | 13.9 | (12.1-16.9) | 0.003 | 0.005 |
| Carnitine | 41.0 | (34.0-48.3) | 42.3 | (36.2-50.5) | 0.03 | 0.08 |
| Betaine | 42.8 | (35.6-54.0) | 46.1 | (37.3-54.7) | 0.46 | 0.11 |
| γ- | 0.63 | (0.53-0.79) | 0.67 | (0.56-0.82) | 0.29 | 0.11 |
| butyrobetaine | | | | | | |
| Croto- | 0.07 | (0.06-0.09) | 0.07 | (0.06-0.10) | 0.34 | 0.73 |
| nobetaine | | | | | | |
| TMAO | 3.63 | (2.46-5.72) | 4.00 | (2.92-6.21) | 0.07 | 0.16 |
| PAGln | 1.75 | (0.96-3.03) | 2.13 | (1.27-3.40) | 0.002 | 0.003 |
| Hippuric | 2.38 | (1.07-4.99) | 2.66 | (1.21-5.00) | 0.99 | 0.51 |
| Acid | | | | | | |
| P-cresol | 15.0 | (7.5-27.6) | 19.7 | (10.6-31.7) | 0.01 | 0.01 |

Sulfate

Abbreviations: TMAO, trimethylamine N-oxide; PAGln, phenylacetylglutamine; IQR, interquartile range
^{a}Serum analyte concentrations are presented as median (IQR)
^{b}Based on multivariable conditional logistic regression adjusted for body mass index (BMI) and prostate-specific antigen (PSA) measured at the time of baseline sample collection
^{c}Trend of increasing ORs was assessed based on quartile medians using the Cochran-Armitage test

Choline (13.9 vs. 13.1 uM, P=0.003) and carnitine (42.3 vs. 41.0 uM, P=0.03) were higher in subjects who developed incident lethal PCa (cases) compared to those who did not (controls). No statistically significant differences were observed among cases and controls with respect to baseline circulating levels of betaine (46.1 vs. 42.8 uM, P=0.46), γ-butyrobetaine (0.67 vs. 0.63 uM, P=0.29), crotonobetaine (0.07 vs. 0.07 uM, P=0.34), or TMAO (4.00 vs. 3.63 uM, P=0.07). Interestingly, baseline levels of the gut-microbiota-derived metabolites PAGln (2.13 uM vs. 1.75 uM, P=0.002) and p-cresol sulfate (19.7 vs. 15.0 uM, P=0.01) were higher in men who subsequently developed lethal PCa, while baseline levels of hippuric acid (2.66 vs. 2.38 uM, P=0.99) were similar between cases and controls.

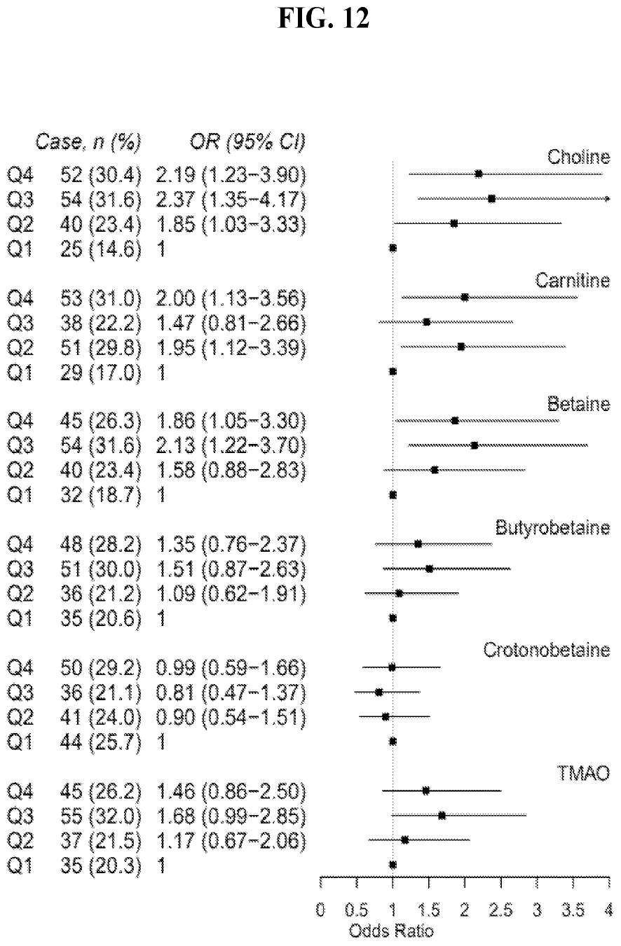

The association between nutrient precursors of the gut microbiota-dependent metabolite TMAO and lethal PCa risk was evaluated across analyte quartiles (FIG. 12). Notably, participants with higher baseline serum levels of choline (Q3 OR: 2.37, 95% CI: 1.35-4.17; Q4 OR: 2.19, 95% CI: 1.23-3.90) or betaine (Q3 OR: 2.13, 95% CI: 1.22-3.70: Q4 OR: 1.86, 95% CI: 1.05-3.30) exhibited increased odds of developing lethal PCa (relative to Q1) in the conditional multivariable logistic regression model adjusted for PSA and BMI. Lethal PCa risk increased across quartiles of choline in a dose-dependent fashion (P-trend: 0.005), a trend not observed with increasing quantiles of betaine (P-trend: 0.11). Higher baseline serum levels of carnitine were inconsistently associated with lethal PCa in this model, with only Q2 (OR: 1.95, 95% CI: 1.12-3.39) and Q4 (OR: 2.00, 95% CI: 1.13-3.56) achieving statistical significance (P-trend: 0.08). None of the other TMA-associated analytes monitored showed significant associations with incident lethal PCa, including γ-butyrobetaine (Q4 OR: 1.35, 95% CI: 0.76-2.37; P-trend: 0.11), crotonobetaine (Q4 OR: 0.99, 95% CI: 0.59-1.66; P-trend: 0.73), and TMAO (Q4 OR: 1.46, 95% CI: 0.86-2.50; P-trend: 0.16).

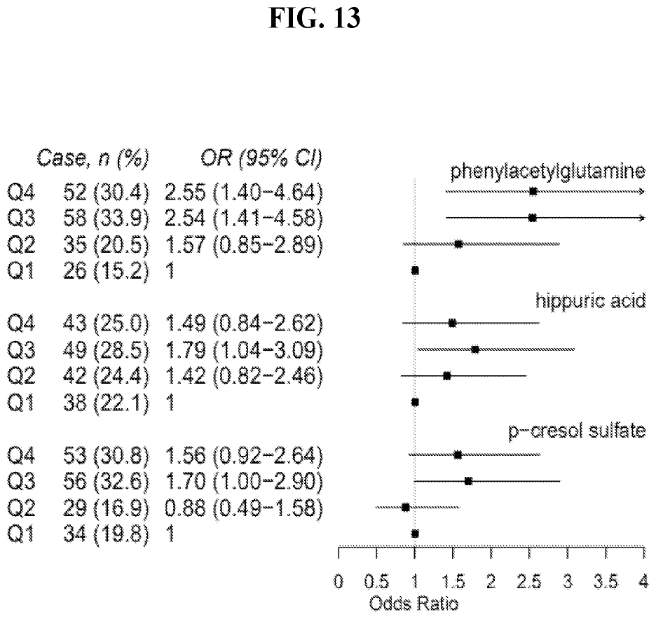

Gut-microbiota-dependent metabolites of aromatic amino acid catabolism were also targeted for analysis (FIG. 13). Notably, participants with higher serum levels of baseline PAGln (Q3: 2.54, 95% CI: 1.414.58: Q4: 2.55, 95% CI: 1.40-4.64) exhibited increased odds (relative to Q1) of developing incident lethal PCa in the conditional regression model adjusted for BMI and PSA. The risk of PCa-specific mortality generally increased across quartiles of PAGln, suggesting a dose-dependent relationship (P-trend: 0.003). Higher levels of hippuric acid (Q3: 1.79, 95% CI: 1.04-3.09: Q4 OR: 1.49, 95% CI: 0.84-2.62; P-trend: 0.51) and p-cresol sulfate (Q3: 1.70, 95% CI: 1.00-2.90; Q4 OR: 1.56, 95% CI: 0.92-2.64 P-trend: 0.01) were inconsistently linked with an increased risk of lethal PCa in the aforementioned model.

The impact of diet, the gut microbiome, and host metabolism on human disease has become increasingly evident.³²⁻³⁵ In this nested case-control study, we show that men with higher levels of choline, betaine, or PAGln have approximately double the odds (relative to Q1) of being later diagnosed with incident lethal PCa. While previous studies have associated choline, TMAO, or both with oncologic risk, this study is the first of its kind to demonstrate that metabolites with an obligatory requirement for gut microbiota (e.g. PAGln) and nutrient precursors (e.g. choline and betaine) that give rise to microbiome-related metabolites are associated with an increased risk of lethal PCa.^{17,18,21}

The significant association of high serum concentrations of PAGln with lethal PCa is intriguing. PAGln is a gut-microbiota-dependent metabolite of dietary phenylalanine, an amino acid consumed through via dietary protein.³⁶ Phenylalanine is released by digestive proteases and primarily absorbed in the small intestine. However, some phenylalanine reaches the large intestine where anaerobic microorganisms play an essential role in PAGln production.³⁷ Nemet et al. employed both genetic and pharmacological studies to definitively demonstrate that this gut-microbiota-derived metabolite signals in hosts through multiple adrenergic receptors (α2A, α2B, and β2 adrenergic receptors).²⁴ In fact, in both multiple cellular systems and animal model studies, numerous cardiovascular-associated effects promoted by PAGln were shown to be attenuated by beta blockers.²⁴ It is therefore highly relevant that sympathetic signaling, specifically through the β2 adrenergic receptor, has been shown to increase PCa aggressiveness by dysregulating apoptosis, enhancing cell migration, augmenting metastatic potential, and accelerating the epithelial-mesenchymal transition observed in some tumors.³⁸ Moreover, a recent meta-analysis examining 16,825 patients associated beta-blocker use with lower PCa-specific mortality (HR: 0.85, 95% CI: 0.77-0.95).³⁹

One of the more striking findings of the present study is that baseline levels of choline and betaine (but not carnitine, γ-butyrobetaine, and crotonobetaine, or TMAO) predicted heightened incident risk of lethal PCa, suggesting that this elevation in oncologic risk is independent of gut-microbiome-related metabolism. A prior report associated increased choline intake with greater lethal PCa risk, while a previous nested case-control study linked higher levels of serum choline with increased odds of any (lethal or non-lethal) prostate cancer diagnosis.^{11,40} Choline may be implicated in oncogenesis given that it serves as a structural component of various phospholipids (including phosphatidylcholine), making this nutrient essential for maintenance of the cell membrane and cancer growth.⁴¹ Additionally, phosphatidylcholine may be metabolized to signaling molecules (such as phosphocholine and diacylglycerol) that transduce mitogenic commands for cell growth.⁴² Choline metabolism has also been observed to be dysregulated in PCa, particularly when choline kinase, an enzyme that facilitates the rate-limiting step in the phosphatidylcholine biosynthesis, is overexpressed.⁴³ These alterations in choline metabolism are particularly relevant, given the clinical utility of using choline-based (¹¹C-choline and ¹⁸F-choline) PET imaging to restage PCa with heightened malignant potential.⁴⁴⁻⁴⁷

Serum elevations in the choline oxidation product, betaine, were also associated with lethal PCa. Betaine serves as a methyl donor in pathways that yield S-adenosylmethionine, a substrate that mediates DNA and histone methylation.⁴⁸ Though our study is the first to positively associate elevated baseline betaine levels with incident lethal PCa, other research evaluating dietary intake concluded that greater betaine consumption offers protection from lethal PCa.⁴⁹ Interestingly, meta-organismal (involving both bacteria and host) betaine and choline metabolism has been shown to alter global DNA methylation patterns, induce epigenetic changes, and even influence behavior in offspring in rodent models.^{50,51}

The discovery that choline, betaine, and PAGln are independently associated with increased risk of lethal PCa has potential implications for PCa risk modification and screening. If these associations are confirmed in independent clinical studies, men with elevated levels of these analytes may be able to consider dietary or pharmacologic risk reduction strategies that modulate meta-organismal choline, betaine, or phenylalanine metabolism. It would also follow that prospective studies might investigate whether assessment of choline, betaine, and PAGln levels help to identify patients for whom earlier initiation of PSA screening might be warranted. A large portion of patients with lethal PCa in this study had GS 7 disease, suggesting levels of these analytes could be considered when weighing eligibility for active surveillance in intermediate-risk patients.

Baseline serum elevations in choline and betaine are associated with incident lethal PCa independent of other nutrients and metabolites in the TMAO pathway when adjusted for PSA and BMI. This association was also shown with higher circulating levels PAGln, a phenylalanine metabolite resulting from gut microbiota metabolism that signals via adrenergic receptors.

### References for Example 3

1. Siegel R L, Miller K D, Jemal A. Cancer statistics, 2020. CA Cancer J Clin. 2020; 70(1):7-30.
2. Irshad S, Bansal M, Castillo-Martin M, et al. A molecular signature predictive of indolent prostate cancer. Sci Transl Med. 2013; 5(202):202ra122.
3. De Stefani E, Ronco A L, Deneo-Pellegrini H, et al. Dietary patterns and risk of advanced prostate cancer: a principal component analysis in Uruguay. Cancer Causes Control. 2010; 21(7):1009-1016.
4. Ambrosini G L, Fritschi L, de Klerk N H, Mackerras D, Leavy J. Dietary patterns identified using factor analysis and prostate cancer risk: a case control study in Western Australia. Ann Epidemiol. 2008; 18(5):364-370.
5. Chan J M, Gann P H, Giovannucci E L. Role of diet in prostate cancer development and progression. J Clin Oncol. 2005; 23(32):8152-8160.
6. Kolonel L N, Hankin J H, Nomura A M. Multiethnic studies of diet, nutrition, and cancer in Hawaii. Princess Takamatsu Symp. 1985; 16:29-40.
7. Hemminki K, Fbrsti A, Khyatti M, Anwar W A, Mousavi M. Cancer in immigrants as a pointer to the causes of cancer. Eur J Public Health. 2014; 24(Suppl 1):64-71.
8. Gann P H. Diet and prostate cancer risk: the embarrassment of riches. Cancer Causes Control. 1998; 9(6):541-543.
9. Richman E L, Kenfield S A, Stampfer M J, Giovannucci E L, Chan J M. Egg, red meat, and poultry intake and risk of lethal prostate cancer in the prostate specific antigen-era: incidence and survival. Cancer Prev Res (Phila). 2011; 4(12):2110-2121.
10. Sinha R, Park Y, Graubard B I, et al. Meat and meat-related compounds and risk of prostate cancer in a large prospective cohort study in the United States. Am J Epidemiol. 2009; 170(9):1165-1177.
11. Richman E L, Kenfield S A, Stampfer M J, et al. Choline intake and risk of lethal prostate cancer: incidence and survival. Am J Clin Nutr. 2012; 96(4):855-863.
12. Huang J, Mondul A M, Weinstein S J, et al. Prospective serum metabolomic profiling of lethal prostate cancer. Int J Cancer. 2019; 145(12):3231-3243.
13. Simó C, Garcia-Cañas V. Dietary bioactive ingredients to modulate the gut microbiota-derived metabolite TMAO. New opportunities for functional food development.

Food Funct. 2020; 11(8):6745-6776.
14. Oliphant K, Allen-Vercoe E. Macronutrient metabolism by the human gut microbiome: major fermentation by-products and their impact on host health. Microbiome. 2019; 7(1):91.doi: 10.1186/s40168-019-0704-8.
15. Wang Z, Klipfell E, Bennett B J, et al. Gut flora metabolism of phosphatidylcholine promotes cardiovascular disease. Nature. 2011; 472(7341):57-63.
16. Tremaroli V, Bäckhed F. Functional interactions between the gut microbiota and host metabolism. Nature. 2012; 489(7415):242-249.
17. Bae S, Ulrich C M, Neuhouser M L, et al. Plasma choline metabolites and colorectal cancer risk in the Women's Health Initiative Observational Study. Cancer Res. 2014; 74(24):7442-7452.
18. Guertin K A, Li X S, Graubard B I, et al. Serum trimethylamine N-oxide, carnitine, choline, and betaine in relation to colorectal cancer risk in the alpha tocopherol, beta carotene cancer prevention study. Cancer Epidemiol Biomarkers Prev. 2017; 26(6):945-952.
19. Liu Z Y, Tan X Y, Li Q J, et al. Trimethylamine N-oxide, a gut microbiota-dependent metabolite of choline, is positively associated with the risk of primary liver cancer: a case-control study. Nutr Metab (Lond). 2018; 15:81. doi: 10.1186/s12986-018-0319-2.
20. Tang W H W, Wang Z, Levison B S, et al. Intestinal microbial metabolism of phosphatidylcholine and cardiovascular risk. N Engl J Med. 2013; 368(17):1575-1584.
21. Mondul A M, Moore S C, Weinstein S J, Karoly E D, Sampson J N, Albanes D. Metabolomic analysis of prostate cancer risk in a prospective cohort: the alpha-tocopherol, beta-carotene cancer prevention (ATBC) study. Int J Cancer. 2015; 137(9):2124-2132.
22. Shafi T, Meyer T W, Hostetter T H, et al. Free levels of selected organic solutes and cardiovascular morbidity and mortality in hemodialysis patients: results from the Retained Organic Solutes and Clinical Outcomes (ROSCO) investigators. PLoS One. 2015; 10(5):e0126048. doi: 10.1371/journal.pone.0126048
23. Cosola C, Rocchetti M T, Cupisti A, Gesualdo L. Microbiota metabolites: pivotal players of cardiovascular damage in chronic kidney disease. Pharmacol Res. 2018; 130:132-142.
24. Nemet I, Saha P P, Gupta N, et al: A cardiovascular disease-linked gut microbial metabolite acts via adrenergic receptors. Cell. 2020; 180(5):862-877.
25. Andriole G L, Crawford E D, Grubb R L, et al. Mortality results from a randomized prostate-cancer screening trial. N Engl J Med. 2009; 360(13):1310-1319.
26. Miller A B, Feld R, Fontana R, et al. Changes in and impact of the death review process in the Prostate, Lung, Colorectal and Ovarian (PLCO) cancer screening trial. Rev Recent Clin Trials. 2015; 10(3):206-211.
27. Koeth R A, Levison B S, Culley M K, et al. γ-Butyrobetaine is a proatherogenic intermediate in gut microbial metabolism of L-carnitine to TMAO. Cell Metab. 2014; 20(5):799-812.
28. Morano C, Zhang X, Fricker L D. Multiple isotopic labels for quantitative mass spectrometry. Anal Chem. 2008; 80(23):9298-9309.
29. Laster H, Seim H. Synthesis of [methyl-14C]crotonobetaine from DL-[methyl-14C]carnitine. J Label Compd Radiopharm. 1996; 38(2):179-186.
30. Wang Z, Bergeron N, Levison B S, et al. Impact of chronic dietary red meat, white meat, or non-meat protein on trimethylamine N-oxide metabolism and renal excretion in healthy men and women. Eur Heart J. 2019; 40(7):583-594.
31. Fabresse N, Uteem I, Lamy E, Massy Z, Armine Larabi I, Alvarez J C. Quantification of free and protein bound uremic toxins in human serum by LC-MS/MS: comparison of rapid equilibrium dialysis and ultrafiltration. Clin Chim Acta. 2020; 507:228-235.
32. Gilbert J A, Blaser M J, Caporaso J G, Jansson J K, Lynch S V, Knight R. Current understanding of the human microbiome. Nat Med. 2018; 24(2):392-400.
33. Koh A, BAckhed F. From association to causality: the role of the gut microbiota and its functional products on host metabolism. Mol Cell. 2020; 78(4):584-596.
34. Attaye I, Pinto-Sietsma S J, Herrema H, Nieuwdorp M. A crucial role for diet in the relationship between gut microbiota and cardiometabolic disease. Annu Rev Med. 2020; 71:149-161.
35. Witkowski M, Weeks T L, Hazen S L. Gut microbiota and cardiovascular Disease. Circ Res. 2020; 127(4):553-570.
36. Poesen R, Claes K, Evenepoel P, et al. Microbiota-Derived Phenylacetylglutamine Associates with Overall Mortality and Cardiovascular Disease in Patients with CKD. J Am Soc Nephrol. 2016; 27(11):3479-3487.
37. Seakins J W. The determination of urinary phenylacetylglutamine as phenylacetic acid. Studies on its origin in normal subjects and children with cystic fibrosis. Clin Chim Acta. 1971; 35(1):121-131.
38. Braadland P R, Ramberg H, Grytli H H, Taskén KA. β-adrenergic receptor signaling in prostate cancer. Front Oncol. 2015; 4:375. doi: 10.3389/fonc.2014.00375
39. Lu H, Liu X, Guo F, et al. Impact of beta-blockers on prostate cancer mortality: a meta-analysis of 16,825 patients. Onco Targets Ther. 2015; 8:985-990.
40. Johansson M, Van Guelpen B, Emil Vollset S, et al. One-carbon metabolism and prostate cancer risk: prospective investigation of seven circulating B vitamins and metabolites. Cancer Epidemiol Biomarkers Prev. 2009; 18(5):1538-43.
41. Glunde K, Bhujwalla Z M, Ronen S M. Choline metabolism in malignant transformation. Nat Rev Cancer. 2011; 11(12):835-848.
42. Zeisel S H, da Costa K A. Choline: an essential nutrient for public health. Nutr Rev. 2009; 67(11):615-623.
43. Challapalli A, Trousil S, Hazell S, et al. Exploiting altered patterns of choline kinase-alpha expression on human prostate tissue to prognosticate prostate cancer. J Clin Pathol. 2015; 68(9):703-709.
44. Ackerstaff E, Pflug B R, Nelson J B, Bhujwalla Z M. Detection of increased choline compounds with proton nuclear magnetic resonance spectroscopy subsequent to malignant transformation of human prostatic epithelial cells. Cancer Res. 2001; 61(9):3599-3603.
45. Treglia G, Ceriani L, Sadeghi R, Giovacchini G, Giovanella L. Relationship between prostate-specific antigen kinetics and detection rate of radiolabelled choline PET/CT in restaging prostate cancer patients: a meta-analysis. Clin Chem Lab Med. 2014; 52(5):725-733.
46. Wondergem M, van der Zant F M, van der Ploeg T, Knol R J J. A literature review of 18F-fluoride PET/CT and 18F-choline or 1 IC-choline PET/CT for detection of bone metastases in patients with prostate cancer. Nucl Med Commun. 2013; 34(10):935-945.
47. Kitajima K, Murphy R C, Nathan M A. Choline PET/CT for imaging prostate cancer: an update. Ann Nucl Med. 2013; 27(7):581-591.
48. Li Z, Vance D E. Phosphatidylcholine and choline homeostasis. J Lipid Res. 2008; 49(6):1187-1194.
49. Han P, Bidulescu A, Barber J R, et al. Dietary choline and betaine intakes and risk of total and lethal prostate cancer in the Atherosclerosis Risk in Communities (ARIC) Study. Cancer Causes Control. 2019; 30(4):343-354.
50. Romano K A, Rey F E. Is maternal microbial metabolism an early-life determinant of health? Lab Anim (NY). 2018; 47(9):239-243.
Romano K A, Martinez-Del Campo A, Kasahara K, et al. Metabolic, epigenetic, and transgenerational effects of gut bacterial choline consumption. Cell Host Microbe. 2017; 22(3):279-290.

### REFERENCES

Action to Control Cardiovascular Risk in Diabetes Study, Gerstein, et al. (2008). Effects of intensive glucose lowering in type 2 diabetes. N Engl J Med 358, 2545-2559.
Amrani and Bradding (2017), Adv Immunol 136, 1-28.
Anfossi and Trovati (1996), Eur J Clin Invest 26, 353-370.
Aron-Wisnewsky and Clement (2016), Nat Rev Nephrol 12, 169-181.
Atwood, et al., (2011), BMC Genomics 12, 14.
Barnett, et al., (1985), Adv Exp Med Biol 192, 97-108.
Barrios, et al. (2015), PLoS One 10, e0134311.
Bilski, et al., 1983, J Cardiovasc Pharmacol 5, 430-437.
Black, H. R., Greenberg, B. H., and Weber, M. A. (2010). The foundation role of beta blockers across the cardiovascular disease spectrum: a year 2009 update. Am J Med 123, S2.
Blazenovic, I., Kind, T., Ji, J., and Fiehn, O. (2018). Software Tools and Approaches for Compound Identification of LC-MS/MS Data in Metabolomics. Metabolites 8.
Brown, J. M., and Hazen, S. L. (2018). Microbial modulation of cardiovascular disease. Nat Rev Microbiol 16, 171-181.
Brusilow et al., (1980). Amino acid acylation: a mechanism of nitrogen excretion in inborn errors of urea synthesis. Science 207, 659-661.
Burnett et al., (2017). Thirty Years of Evidence on the Efficacy of Drug Treatments for Chronic Heart Failure With Reduced Ejection Fraction: A Network Meta-Analysis. Circ Heart Fail 10.
Campbell, A. P., and Smrcka, A. V. (2018). Targeting G protein-coupled receptor signalling by blocking G proteins. Nat Rev Drug Discov 17, 789-803.
Ciccarelli et al., (2013). Adrenergic receptors and metabolism: role in development of cardiovascular disease. Front Physiol 4, 265.
Colman, R. W. (1990). Platelet receptors. Hematol Oncol Clin North Am 4, 27-42.
da Silva, et al., (2015). Illuminating the dark matter in metabolomics. Proc Natl Acad Sci USA 112, 12549-12550.
Devlin et al., (2016). Modulation of a Circulating Uremic Solute via Rational Genetic Manipulation of the Gut Microbiota. Cell Host Microbe 20, 709-715.
Dickert et al., (2002). Molecular characterization of phenyllactate dehydratase and its initiator from Clostridium sporogenes. Mol Microbiol 44, 49-60.
Dodd et al. (2017). A gut bacterial pathway metabolizes aromatic amino acids into nine circulating metabolites. Nature 551, 648-652.
Duckworth et al. (2009). Glucose control and vascular complications in veterans with type 2 diabetes. N Engl J Med 360, 129-139.
Elliott et al. (2015). Urinary metabolic signatures of human adiposity. Sci Transl Med 7, 285ra262.
Elsden, et al., (1976). The end products of the metabolism of aromatic amino acids by Clostridia. Arch Microbiol 107, 283-288.
Fawzi, et al., (2001). SCH-202676: An allosteric modulator of both agonist and antagonist binding to G protein-coupled receptors. Mol Pharmacol 59, 30-37.
Fu, et al., (2017). Insulin and beta Adrenergic Receptor Signaling: Crosstalk in Heart. Trends Endocrinol Metab 28, 416-427.
Gerstein, et al. (2001). Albuminuria and risk of cardiovascular events, death, and heart failure in diabetic and nondiabetic individuals. JAMA 286, 421-426.
Gonzalez-Guardia, et al. (2015). Effects of the Mediterranean diet supplemented with coenzyme q10 on metabolomic profiles in elderly men and women. J Gerontol A Biol Sci Med Sci 70, 78-84.
Guo et al., Science. 2019 Dec. 13; 366(6471).
Gupta, et al., (2015). Deubiquitinases Modulate Platelet Proteome Ubiquitination, Aggregation, and Thrombosis. Arterioscler Thromb Vasc Biol 35, 2657-2666.
Gupta, et al., (2004). Patterns of beta-blocker utilization in patients with chronic heart failure: experience from a specialized outpatient heart failure clinic. Am Heart J 147, 79-83.
Hein, L. (2006). Adrenoceptors and signal transduction in neurons. Cell Tissue Res 326, 541-551.
Hein, L., and Kobilka, B. K. (1997). Adrenergic Receptors From Molecular Structure to in vivo function. Trends Cardiovasc Med 7, 137-145.
Ilardi, et al. (2018). Effects of Carvedilol Versus Metoprolol on Platelet Aggregation in Patients With Acute Coronary Syndrome: The PLATE-BLOCK Study. Am J Cardiol 122, 6-11.
Jonsson, A. L., and Backhed, F. (2017). Role of gut microbiota in atherosclerosis. Nat Rev Cardiol 14, 79-87.
Koeth, et al., (2013). Intestinal microbiota metabolism of L-carnitine, a nutrient in red meat, promotes atherosclerosis. Nat Med 19, 576-585.
Koh, et al. (2018). Microbially Produced Imidazole Propionate Impairs Insulin Signaling through mTORC1. Cell 175, 947-961 e917.
Koopen, et al., (2016). Human microbiome as therapeutic intervention target to reduce cardiovascular disease risk. Curr Opin Lipidol 27, 615-622.
Koryakina, et al., (2012). Effects of the beta-agonist, isoprenaline, on the down-regulation, functional responsiveness and trafficking of beta2-adrenergic receptors with N-terminal polymorphisms. Cell Biol Int 36, 1171-1183.
Lefkowitz, et al., (2000). Catecholamines, cardiac beta-adrenergic receptors, and heart failure. Circulation 101, 1634-1637.
Leitinger, B. (2011). Transmembrane collagen receptors. Annu Rev Cell Dev Biol 27, 265-290.
Lewandowicz, et al., (2006). The ‘allosteric modulator’ SCH-202676 disrupts G protein-coupled receptor function via sulphydryl-sensitive mechanisms. Br J Pharmacol 147, 422-429.
Li, et al., (2018). Untargeted metabolomics identifies trimethyllysine, a TMAO-producing nutrient precursor, as a predictor of incident cardiovascular disease risk. JCI Insight 3.
Lloyd-Price, et al. (2019). Multi-omics of the gut microbial ecosystem in inflammatory bowel diseases. Nature 569, 655-662.
Loo, et al., (2018). Characterization of metabolic responses to healthy diets and association with blood pressure: application to the Optimal Macronutrient Intake Trial for Heart Health (OmniHeart), a randomized controlled study. Am J Clin Nutr 107, 323-334.
Milligan, G., and Kostenis, E. (2006). Heterotrimeric G-proteins: a short history. Br J Pharmacol 147 Suppl 1, S46-55.
Moldave, K., and Meister, A. (1957). Synthesis of phenylacetylglutamine by human tissue. J Biol Chem 229, 463-476.
O'Rourke, et al., (1994). Characterization of [3H]RX821002 binding to alpha-2 adrenergic receptor subtypes. J Pharmacol Exp Ther 268, 1362-1367.
Petrikova, et al., (2005) Carvedilol—a beta-blocker with considerable antiaggregatory effect on human blood platelets. Bratisl Lek Listy 106, 20-25.
Piarulli, et al., (2009). Relationship between glyco-oxidation, antioxidant status and microalbuminuria in type 2 diabetic patients. Diabetologia 52, 1419-1425.
Poesen, et al., (2016). Microbiota-Derived Phenylacetylglutamine Associates with Overall Mortality and Cardiovascular Disease in Patients with CKD. J Am Soc Nephrol 27, 3479-3487.
Rockman, et al., (2002). Seven-transmembrane-spanning receptors and heart function. Nature 415, 206-212.
Schroder, et al. (2010). Deconvolution of complex G protein-coupled receptor signaling in live cells using dynamic mass redistribution measurements. Nat Biotechnol 28, 943-949.
Schroder, et al. (2011). Applying label-free dynamic mass redistribution technology to frame signaling of G protein-coupled receptors noninvasively in living cells. Nat Protoc 6, 1748-1760.
Shafi, et al., (2015). Free Levels of Selected Organic Solutes and Cardiovascular Morbidity and Mortality in Hemodialysis Patients: Results from the Retained Organic Solutes and Clinical Outcomes (ROSCO) Investigators. PLoS One 10, e0126048.
Shafi, et al., (2017). Results of the HEMO Study suggest that p-cresol sulfate and indoxyl sulfate are not associated with cardiovascular outcomes. Kidney Int 92, 1484-1492.
Skye, et al., (2018). Microbial Transplantation With Human Gut Commensals Containing CutC Is Sufficient to Transmit Enhanced Platelet Reactivity and Thrombosis Potential. Circ Res 123, 1164-1176.
Small, et al., (2003). Pharmacology and physiology of human adrenergic receptor polymorphisms. Annu Rev Pharmacol Toxicol 43, 381-411.
Srikanthan, S., Li, W., Silverstein, R. L., and McIntyre, T. M. (2014). Exosome poly-ubiquitin inhibits platelet activation, downregulates CD36 and inhibits pro-atherothombotic cellular functions. J Thromb Haemost 12, 1906-1917.
Tang, et al., (2013). Intestinal microbial metabolism of phosphatidylcholine and cardiovascular risk. N Engl J Med 368, 1575-1584.
Tsugawa, et al., (2015). MS-DIAL: data-independent MS/MS deconvolution for comprehensive metabolome analysis. Nat Methods 12, 523-526.
Urpi-Sarda, et al. (2019). Non-targeted metabolomic biomarkers and metabotypes of type 2 diabetes: A cross-sectional study of PREDIMED trial participants. Diabetes Metab 45, 167-174.
Wang, et al., (2013). Metabonomic profiling of serum and urine by (1)H NMR-based spectroscopy discriminates patients with chronic obstructive pulmonary disease and healthy individuals. PLoS One 8, e65675.
Wang et al., (2011). Gut flora metabolism of phosphatidylcholine promotes cardiovascular disease. Nature 472, 57-63.
Wang, et al., (2007). Protein carbamylation links inflammation, smoking, uremia and atherogenesis. Nat Med 13, 1176-1184.
Webster, et al., (1976). Identification of separate acyl-CoA:glycine and acyl-CoA:L-glutamine N-acyltransferase activities in mitochondrial fractions from liver of rhesus monkey and man. J Biol Chem 251, 3352-3358.
Witte, et al. (2018). Mortality Reduction Associated With beta-Adrenoceptor Inhibition in Chronic Heart Failure Is Greater in Patients With Diabetes. Diabetes Care 41, 136-142.
Yang et al. (1996). Noninvasive probing of citric acid cycle intermediates in primate liver with phenylacetylglutamine. Am J Physiol 270, E882-889.
Zhang, et al. (2018). Functional Metabolomics Characterizes a Key Role for N-Acetylneuraminic Acid in Coronary Artery Diseases. Circulation 137, 1374-1390.
Zhang, R., and Xie, X. (2012). Tools for GPCR drug discovery. Acta Pharmacol Sin 33, 372-384.
Zhu, et al., (2016). Gut Microbial Metabolite TMAO Enhances Platelet Hyperreactivity and Thrombosis Risk. Cell 165, 111-124.

Although only a few exemplary embodiments have been described in detail, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure.

What is claimed is:

1. A method performing an activity based on the level of at least phenylacetyl glutamine (PAG) in a sample from a subject comprising:
a) determining the level of at least one compound in a sample from a subject, wherein said at least one compound comprises PAG, and wherein optionally said subject is chronic kidney disease (CKD) free and/or has type II diabetes; and
b) performing at least one of the following activities:
i) identifying increased levels of said at least one compound in said sample compared to control levels, and treating said subject with:
A) a first agent or first procedure that treats cardiovascular disease (CVD), asthma, heart failure, and/or thrombosis, wherein said subject is CKD free and/or has diabetes;
B) a second agent selected from: a beta-adrenergic blocking agent, an alpha 2 adrenergic receptor agonist, an alpha 2 adrenergic receptor antagonist, and an antibiotic or antibiotic cocktail; and/or
C) a third agent or second procedure selected from: a prostate cancer therapeutic, brachytherapy, radiation therapy, and prostatectomy,
ii) generating and/or transmitting a report that indicates that levels of said at least one compound are elevated in said sample compared to control levels, and that said subject has, or is at risk for, CVD, thrombosis, asthma, lethal prostate cancer, and/or heart failure and is treatable by: A) said beta-adrenergic blocking agent, and/or B) said alpha 2 adrenergic receptor agonist, and/or C) said an alpha 2 adrenergic receptor antagonist and/or D) said antibiotic or antibiotic cocktail; and/or E) said third agent or second procedure;
iii) generating and/or transmitting a report that indicates that levels of said at least one compound are elevated in said sample compared to control levels, and that said subject is in need of said first agent, said second agent, and/or said first procedure, and/or said third agent or second procedure;
iv) generating and/or transmitting a report for a subject that is CKD free and/or has diabetes, wherein said report indicates that levels of said at least one compound are elevated in said sample compared to control levels, and that said subject has, or is at risk for, cardiovascular disease, thrombosis, lethal prostate cancer, and/or heart failure; and
v) characterizing said subject who is CKD free and/or has diabetes as having, or at risk for, CVD, thrombosis, and/or heart failure, or characterizing said subject as having, or at risk for, lethal prostate cancer, based on finding elevated levels of said at least one compound in said sample compared to a control levels.

2. The method of claim 1, wherein said at least one compound further comprises trimethylamine-n-oxide (TMAO).

3. The method of claim 1, wherein: i) said subject has heart failure, asthma, or cardiovascular disease, and wherein said at least one compound further comprises N6-trimethyl-lysine (TML) and/or TMAO, or ii) said subject has prostate cancer, and said at least one compound further comprises PSA, choline, and/or betaine.

4. The method of claim 1, wherein said at least one compound further comprises TMAO and TML.

5. The method of claim 1, wherein said sample is selected from the group consisting of:
a plasma sample, a serum sample, a whole blood sample, and a urine sample.

6. The method of claim 1, wherein said determining comprises detecting said at least one compound with an analytical device selected from: a mass spectrometer, NMR spectrometer, and a UV/Vis spectrometer.

7. The method of claim 1, wherein said determining comprises contacting said sample with an anti-PAG antibody or PAG binding fragment thereof.

8. The method of claim 1, wherein said a beta-adrenergic blocking agent is selected from: acebutolol hydrochloride, atenolol, betaxolol hydrochloride, bisoprolol fumarate, carteolol hydrochloride, esmolol hydrochloride, metoprolol, penbutolol sulfate, nadolol, nebivolol, pindolol, propranolol, timolol maleate, sotalol hydrochloride, carvedilol, and labetalol hydrochloride.

9. The method of claim 1, wherein said alpha 2 adrenergic receptor agonist is selected from: Clonidine, Dexmedetomidine, Fadolmidine, Guanfacine, Guanabenz, Guanoxabenz, Guanethidine, Xylazine, Tizanidine, Medetomidine, Methyldopa, Methylnorepinephrine, Norepinephrine, (R)-3-nitrobiphenyline, amitraz, Detomidine, Lofexidine,and Medetomidine.

10. The method of claim 1, wherein said alpha 2 adrenergic receptor antagonist is selected from: atipamezole, efaroxan, idazoxan, yohimbine, rauwolscine, and phentolamine.

11. The method of claim 1, wherein said first agent is selected from the group consisting of: an anticoagulant, an antiplatelet agent, an ACE Inhibitor, an angiotensin II receptor blocker, an angiotensin-receptor neprilysin inhibitor, a calcium channel blocker, a cholesterol-lowering medication, a statin, a digitalis preparation, a diuretic, and a vasodilator.

12. The method of claim 1, wherein said antibiotic is at least one antibiotic selected from the group consisting of: metronidazole, ciprofloxacin, neomycin, vancomycin, amoxicillin, and a broad spectrum antibiotic; and wherein said subject does not have an active infection.

13. The method of claim 1, wherein said subject is a human with, or at risk for, prostate cancer, cardiovascular disease, asthma, or heart failure.

14. A method of treatment comprising:
a) identifying a subject as having increased levels of at least one compound compared to control levels, wherein said at least one compound comprises phenylacetyl glutamine (PAG); and
b) treating said subject with at least one of the following:
i) a first agent or procedure that treats cardiovascular disease (CVD), heart failure, asthma, and/or thrombosis, wherein said subject is chronic kidney disease (CKD) free and/or has diabetes,
ii) a second agent selected from: a beta-adrenergic blocking agent, an alpha 2 adrenergic receptor agonist, and an alpha 2 adrenergic receptor antagonist,
iii) an antibiotic or antibiotic cocktail, wherein said subject does not have an active infection;
iv) a third agent or second procedure selected from: a prostate cancer therapeutic, brachytherapy, radiation therapy, and prostatectomy.

15. The method of claim 14, wherein said identifying comprises receiving a report that said subject has increased PAG levels compared to a control.

16. The method of claim 14, wherein said at least one compound further comprises PSA, choline, and/or betaine, and said subject has prostate cancer.

17. The method of claim 14, wherein said subject is a human having heart failure, asthma, cardiovascular disease, or prostate cancer.

18. The method of claim 17, wherein said a beta-adrenergic blocking agent is selected from: acebutolol hydrochloride, atenolol, betaxolol hydrochloride, bisoprolol fumarate, carteolol hydrochloride, esmolol hydrochloride, metoprolol, penbutolol sulfate, nadolol, nebivolol, pindolol, propranolol, timolol maleate, sotalol hydrochloride, carvedilol, and labetalol hydrochloride.

19. The method of claim 14, wherein said alpha 2 adrenergic receptor agonist is selected from: Clonidine, Dexmedetomidine, Fadolmidine, Guanfacine, Guanabenz, Guanoxabenz, Guanethidine, Xylazine, Tizanidine, Medetomidine, Methyldopa, Methylnorepinephrine, Norepinephrine, (R)-3-nitrobiphenyline, amitraz, Detomidine, Lofexidine,and Medetomidine.

20. The method of claim 14, wherein said alpha 2 adrenergic receptor antagonist is selected from: atipamezole, efaroxan, idazoxan, yohimbine, rauwolscine, and phentolamine.

21. The method of claim 14, wherein said first agent is selected from the group consisting of: an anticoagulant, an antiplatelet agent, an ACE Inhibitor, an angiotensin II receptor blocker, an angiotensin-receptor neprilysin inhibitor, a calcium channel blocker, a cholesterol-lowering medication, a statin, a digitalis preparation, a diuretic, and a vasodilator.

22. The method of claim 14, wherein said antibiotic is at least one antibiotic selected from the group consisting of: metronidazole, ciprofloxacin, neomycin, vancomycin, amoxicillin, and a broad spectrum antibiotic; and wherein said subject does not have an active infection.

23. The method of claim 14, wherein said subject has heart failure, asthma, or cardiovascular disease, and wherein said at least one compound further comprises trimethylamine-n-oxide (TMAO) and/or N6-trimethyl-lysine (TML)

24. A method of detecting at least one compound in a sample comprising:
a) obtaining a sample, wherein said sample is from a human subject who is optionally chronic kidney disease (CKD) free and/or has diabetes; and
b) treating said sample under conditions such that the concentration of at least one compound is determined, wherein said at least one compound comprises phenylacetyl glutamine (PAG).

25. The method of claim 24, wherein said concentration of said PAG in said sample is determined by mass spectrometry.

26. The method of claim 24, wherein said wherein said subject has heart failure, asthma, cardiovascular disease, or prostate cancer.

27. The method of claim 24, wherein said at least one compound further comprises trimethylamine-n-oxide (TMAO) and/or N6-trimethyl-lysine (TML) and/or PSA and/or choline, and/or betaine.

28. A system or kit comprising:
a) a report for a subject with cardiovascular disease, heart failure, asthma, thrombosis, and/or prostate cancer, wherein said report indicates that said patient has elevated levels of at least one compound, wherein said at least one compound comprises phenylacetyl glutamine (PAG); and
b) at least one of the following:
i) a first agent that treats CVD, heart failure, and/or thrombosis, wherein said subject is chronic kidney disease (CKD) free and/or has diabetes,
ii) a second agent selected from: a beta-adrenergic blocking agent, an alpha 2 adrenergic receptor agonist, and an alpha 2 adrenergic receptor antagonist,
iii) an antibiotic or antibiotic cocktail, wherein said subject does not have an active infection, and
iv) a prostate cancer therapeutic.

29. The system of claim 28, wherein said subject is a human.

30. The system of claim 28, wherein said a beta-adrenergic blocking agent is selected from: acebutolol hydrochloride, atenolol, betaxolol hydrochloride, bisoprolol fumarate, carteolol hydrochloride, esmolol hydrochloride, metoprolol, penbutolol sulfate, nadolol, nebivolol, pindolol, propranolol, timolol maleate, sotalol hydrochloride, carvedilol, and labetalol hydrochloride.

31. The system of claim 28, wherein said alpha 2 adrenergic receptor agonist is selected from: Clonidine, Dexmedetomidine, Fadolmidine, Guanfacine, Guanabenz, Guanoxabenz, Guanethidine, Xylazine, Tizanidine, Medetomidine, Methyldopa, Methylnorepinephrine, Norepinephrine, (R)-3-nitrobiphenyline, amitraz, Detomidine, Lofexidine,and Medetomidine.

32. The system of claim 28, wherein said alpha 2 adrenergic receptor antagonist is selected from: atipamezole, efaroxan, idazoxan, yohimbine, rauwolscine, and phentolamine.

33. The system of claim 28, wherein said first agent is selected from the group consisting of: an anticoagulant, an antiplatelet agent, an ACE Inhibitor, an angiotensin II receptor blocker, an angiotensin-receptor neprilysin inhibitor, a calcium channel blocker, a cholesterol-lowering medication, a statin, a digitalis preparation, a diuretic, and a vasodilator.

34. The system of claim 28, wherein said antibiotic is at least one antibiotic selected from the group consisting of: metronidazole, ciprofloxacin, neomycin, vancomycin, amoxicillin, and a broad spectrum antibiotic; and wherein said subject does not have an active infection.

35. The system of claim 28, wherein said at least one compound further comprises trimethylamine-n-oxide (TMAO) and/or N6-trimethyl-lysine (TML) and/or PSA and/or choline, and/or betaine.

36. A method of detecting phenylacetyl glutamine (PAG) in a serum or plasma sample comprising:
a) obtaining a plasma or serum sample, wherein said plasma or serum sample is from a human subject who is chronic kidney disease (CKD) free and/or has type II diabetes;
b) introducing at least a portion of said plasma or serum sample into an analytical device under conditions such that the concentration of PAG present in said plasma or serum sample is determined,
wherein said analytical device comprises: i) an NMR spectrometer, a UVNis spectrometer, or a mass spectrometer, ii) equipment to provide physical separation of said PAG prior to determining said concentration, and iii) an anti-PAG antibody based detection system; and
c) graphically displaying said subject's risk of having a disease as higher than normal if said level of PAG in said plasma or serum sample is higher than a first minimum value, wherein said minimum value is at least 4.9 μM or at least 3.8 μM, wherein said disease is selected from the group consisting of heart failure, asthma, cardiovascular disease, or thrombosis.

37. The method of claim 36, further comprising determining the level of trimethylamine N-oxide (TMAO) in a sample from a subject, and graphically displaying said subject's risk of said disease as higher than normal when said TMAO level is higher than a second minimum value, wherein said second minimum value is at least 2.2 μM.

38. The method of claim 36, wherein said second minimum value is at least 2.5 μM.

39. The method of claim 36, wherein said second minimum value is at least 3.0 μM.

40. The method of claim 36, wherein said second minimum value is at least 4.0 μM.

41. The method of claim 36, wherein said second minimum value is at least 5.0 μM.

42. The method of claim 36, further comprising determining the level of N6-trimethyl-lysine (TML) in a sample from a subject, and graphically displaying said subject's risk of said disease as higher than normal when said TML level is higher than a second minimum value, wherein said second minimum value is at least 0.4 μM.

43. The method of claim 42, wherein said second minimum value is at least 0.5 μM.

44. The method of claim 42, wherein said second minimum value is at least 0.6 μM.

45. The method of claim 42, wherein said second minimum value is at least 0.7 μM.

46. The method of claim 42, wherein said second minimum value is at least 0.8 μM.

48. A method of detecting at least three compounds in a sample comprising:
a) obtaining a sample, wherein said sample is from a human subject; and
b) treating said sample under conditions such that the concentration of at least the following three compounds is determined: phenylacetyl glutamine (PAG), N6-trimethyl-lysine (TML), and trimethylamine N-oxide (TMAO).

49. The method of claim 48, further comprising: c) graphically displaying said subject's risk of having a disease as higher than normal if all three are present:
i) said level of PAG in said sample is higher than a control PAG value from the general population or disease free group;
ii) said TMAO level in said sample is higher than a control TMAO value from the general population or a disease free group; and
iii) said TML level is in said sample is higher than a control TML value from the general population or a disease free group; and
wherein said disease is selected from the group consisting of heart failure, cardiovascular disease, kidney disease, asthma, or thrombosis.

50. The method of claim 48, further comprising: c) graphically displaying said subject's risk of having a disease as higher than normal if all three are present:
i) said level of PAG in said sample is higher than a first minimum value, wherein said minimum value is at least 3.9 or 4.9 M,
ii) said TMAO level is higher than a second minimum value, wherein said second minimum value is at least 2.2 or 5.0 μM; and
iii) said TML level is higher than a third minimum value, wherein said second minimum value is at least 0.4 μM or 0.6; and
wherein said disease is selected from the group consisting of heart failure, cardiovascular disease, kidney disease, asthma, or thrombosis.

51. A method of detecting phenylacetyl glutamine (PAG) in a serum or plasma sample comprising:
a) obtaining a plasma or serum sample, wherein said plasma or serum sample is from a human subject who is optionally chronic kidney disease (CKD) free and/or has type II diabetes;
b) introducing at least a portion of said plasma or serum sample into an analytical device under conditions such that the concentration of PAG present in said plasma or serum sample is determined,
wherein said analytical device comprises: i) an NMR spectrometer, a UVNis spectrometer, or a mass spectrometer, ii) equipment to provide physical separation of said PAG prior to determining said concentration; and iii) an anti-PAG antibody based detection system; and
c) graphically displaying said subject's risk of having a disease as higher than normal if said level of PAG in said plasma or serum sample is higher than a first minimum value, wherein said minimum value is at least 1.0 μM or at least 3.0 μM, wherein said disease is selected from the group consisting of prostate cancer and lethal prostate cancer.

52. The method of claim 51, further comprising determining the level of choline in a sample from a subject, and graphically displaying said subject's risk of said disease as higher than normal when said choline level is higher than a second minimum value, wherein said second minimum value is at least 11.0 μM.

53. The method of claim 51, wherein said second minimum value is at least 13.0 μM.

54. The method of claim 51, wherein said second minimum value is at least 14.0 μM.

55. The method of claim 51, wherein said second minimum value is at least 15.0 μM.

56. The method of claim 51, wherein said second minimum value is at least 17.0 μM.

57. The method of claim 51, further comprising determining the level of betaine in a sample from a subject, and graphically displaying said subject's risk of said disease as higher than normal when said betaine level is higher than a second minimum value, wherein said second minimum value is at least 35.6 μM.

58. The method of claim 57, wherein said second minimum value is at least 42.8 μM.

59. The method of claim 57, wherein said second minimum value is at least 54.0 μM.

60. The method of claim 57, wherein said second minimum value is at least 54.7 μM.

61. The method of claim 57, wherein said second minimum value is at least 55.0 μM.

62. A method of detecting at least three or four compounds in a sample comprising:
a) obtaining a sample, wherein said sample is from a human subject; and
b) treating said sample under conditions such that the concentration of at least the following three compounds is determined: phenylacetyl glutamine (PAG), choline, and betaine, and optionally a fourth compound: prostate specific antigen (PSA).

63. The method of claim 62, further comprising: c) graphically displaying said subject's risk of having a disease as higher than normal if all three are present:
i) said level of PAG in said sample is higher than a control PAG value from the general population or disease free group;
ii) said choline level in said sample is higher than a control choline value from the general population or a disease free group; and
iii) said betaine level is in said sample is higher than a control TML value from the general population or a disease free group; and
iv) and optionally, said PSA level in said sample is higher than a control PSA value from the general population or disease free group; and
wherein said disease is selected from the group consisting of prostate cancer and lethal prostate cancer.

64. The method of claim 63, further comprising: c) graphically displaying said subject's risk of having a disease as higher than normal if all three are present:
i) said level of PAG in said sample is higher than a first minimum value, wherein said minimum value is at least 3.9 or 4.9 M,
ii) said choline level is higher than a second minimum value, wherein said second minimum value is at least 11.0 or 13.0 μM; and
iii) said betaine level is higher than a third minimum value, wherein said second minimum value is at least 35.6 μM or 42.8; and
wherein said disease is selected from the group consisting of prostate cancer and lethal prostate cancer.
